SEQUENCE LISTING (10 sequences)

SEQ ID NO: 1 - DNA, 18 bp, synthetic construct
  source 1..18: mol_type = other DNA
ggagacgtgaccgtctct

SEQ ID NO: 2 - DNA, 16 bp, synthetic construct
  source 1..16: mol_type = other DNA
ggagacggacgtctcc

SEQ ID NO: 3 - DNA, 20 bp, synthetic construct
  source 1..20: mol_type = other DNA
tctgcagcctagtgggaagg

SEQ ID NO: 4 - DNA, 20 bp, synthetic construct
  source 1..20: mol_type = other DNA
cctgagtgaattacccagga

SEQ ID NO: 5 - DNA, 20 bp, synthetic construct
  source 1..20: mol_type = other DNA
ggggaagtttcggcccaagt

SEQ ID NO: 6 - DNA, 20 bp, synthetic construct
  source 1..20: mol_type = other DNA
tattccacttgaactcccca

SEQ ID NO: 7 - DNA, 20 bp, synthetic construct
  source 1..20: mol_type = other DNA
cggaggtgcggcgagcagga

SEQ ID NO: 8 - DNA, 22 bp, synthetic construct
  source 1..22: mol_type = other DNA
gagttaggcagggatattcacc

SEQ ID NO: 9 - DNA, 19 bp, synthetic construct
  source 1..19: mol_type = other DNA
ctgccccggttaatttgca

SEQ ID NO: 10 - DNA, 20 bp, synthetic construct
  source 1..20: mol_type = other DNA
acggctcattctgctccccc

(19) United States
(12) Patent Application Publication
(10) Pub. No.: US 2026/0321355 A1
(43) Pub. Date: Oct. 15, 2026

(54) BIOENGINEERING AND MACHINE LEARNING FRAMEWORK FOR COMPLEX TISSUE DEVELOPMENT

(71) Applicant: President and Fellows of Harvard College, Cambridge, MA (US)

(72) Inventors: Sharad Ramanathan, Cambridge, MA (US); Giridhar Mandayam Anand, Cambridge, MA (US); Yusuf Ilker YAMAN, Cambridge, MA (US); Roya Eskandari Huang, Cambridge, MA (US); Tianlei He, Cambridge, MA (US); Heitor Megale, Cambridge, MA (US)

(73) Assignee: President and Fellows of Harvard College, Cambridge, MA (US)

(21) Appl. No.: 19/483,312

(22) PCT Filed: May 10, 2024
(86) PCT No.: PCT/US2024/028838

§ 371 Date: Nov. 11, 2025

Related U.S. Application Data
(60) Provisional application No. 63/465,844, filed on May 11, 2023.
(60) Provisional application No. 63/523,064, filed on Jun. 24, 2023.

(51) Int. Cl. G16B 40/30; C12N 5/071

(52) U.S. Cl. CPC G16B 40/30; C12N 5/0697; C12N 2501/15; C12N 2501/155; C12N 2501/415; C12N 2510/00

(57) ABSTRACT

Provided herein is a bioengineering and machine learning framework for complex tissue development, which can be implemented for the identification and/or assessment of gene and/or drug therapies.

### RELATED APPLICATION

This application claims the benefit under 35 U.S.C. § 119(e) of U.S. Provisional Application No. 63/465,844, filed on May 11, 2023, and U.S. Provisional Application No. 63/523,064, filed on Jun. 24, 2023, the content of each of which is incorporated by reference herein in its entirety.

### GOVERNMENT LICENSE RIGHTS

This invention was made with government support under MH123948 and HD100036 awarded the National Institutes of Health. The government has certain rights in the invention.

### BACKGROUND

Current model organisms inadequately mirror human tissue/organ development and disease, and ethical constraints limit many direct human studies, such as human brain and spinal tissue studies. Current brain organoids, for example, fail to replicate the forebrain's spatial patterning, lacking essential features such as a midline, choroid plexus, stable lumen, cortical hem, and Cajal Retzius cells produced by the hem, which are all vital for accurate cortical development. Likewise, current organoids fail to replicate the simultaneous development of the musculoskeletal system and spinal cord, with well-defined dorsoventral and anterior-posterior axes. The inability of in vitro models to mirror critical features of human development and disease has hindered mechanistic understanding of normal and aberrant human development. It is important to develop in vitro models that accurately represent human tissue microenvironments, organization, and function. Accurately reproducing the attributes of a developing organ or other complex tissue and the ability to perturb the genetic network or cellular function at scale is critical for deciphering mechanisms underpinning human development and disease.

### SUMMARY

Provided herein, in some aspects, is a platform technology that combines bioengineering with machine learning to develop hundreds of organoids (stem cell aggregates) that reproducibly undergo deterministic patterning to recapitulate critical features of developing human tissues, including their patterning, morphology, and regional cellular organization. Using a combination of single-cell and in situ sequencing, live imaging, and perturbations, for example, the technology provide herein can be used to characterized developing organoids and uncovered distinct roles for signaling pathways during morphogenesis. The technology provided herein, in some aspects, also enables dynamic measurements and temporally controlled cell type-specific genetic and/or chemical perturbations in a scalable, rapid, high throughput, low-cost manner, which has not been possible with a human relevant model in vitro or in vivo.

Some aspects relate to bioengineering methods for producing a target patterned tissue characteristic of a complex tissue (e.g., human tissue), the methods comprising: coupling organoids to each other based on their relative spatial arrangement; and culturing the organoids under conditions that promote development of the target patterned tissue. In some embodiments, the spatial arrangement is determined using a machine learning and/or high-dimensional statistical model described herein.

Other aspects of the disclosure relate to methods of using a trained machine learning (ML) model to identify a spatial arrangement of stem cell aggregates, the spatial arrangement for promoting development of the stem cell aggregates into target patterned tissue, the methods comprising: using at least one processor to perform: accessing the trained ML model, the trained ML model being trained to predict, using a feature vector generated for an input spatial arrangement of the stem cell aggregates, a patterning metric associated with the input spatial arrangement; and identifying a spatial arrangement of stem cell aggregates that optimizes the patterning metric by using a stochastic optimization technique in which different spatial arrangements of stem cell aggregates are evaluated using their respective patterning metrics as predicted by applying the trained ML model to feature vectors generated for the different spatial arrangements.

Still other aspects relate to cell micropatterning methods, comprising: micropatterning stem cells on a biocompatible substrate, in a predetermined spatial arrangement, wherein the predetermined spatial arrangement promotes development of organoids into a target patterned tissue. In some embodiments, the spatial arrangement is determined using a machine learning and/or high-dimensional statistical model described herein.

In some embodiments, the methods further comprises: using photolithography to fabricate the substrate with microwells to hold signal-secreting beads at desired locations; using a stamp comprising water channels to microprint shape (e.g., a circle or polygonal shape; e.g., about 50 to 150 shapes) of extracellular matrix protein on the substrate; depositing stem cells onto the substrate; and exposing the stem cells to media containing extracellular matrix protein (e.g., Matrigel™) to produce cysts (e.g., epithelial cysts), each enclosing a respective lumen. In some embodiments, a method further comprises depositing signal-secreting beads into the microwells, thereby generating signaling gradients on the substrate (see, e.g., FIG. 13A). In some embodiments, the relative spatial arrangement of these microwells is determined by iterative optimization using these bioengineering methods in conjunction with machine learning and/or high-dimensional statistical analyses.

Yet other aspects relate to rapid, high-throughput screening methods, comprising: on a biocompatible multiwell substrate, or on multiple biocompatible multiwell substrates, coupling organoids to each other based on their relative spatial arrangement, optionally wherein the substrate is fabricated using the method above; culturing the organoids under conditions that promote development of a target patterned tissue (e.g., complex tissue); exposing the organoids to cell signals, and/or to genetic and/or chemical perturbation; and assessing changes in development of, or in one or more characteristic of, the organoids.

Yet other aspects relate to rapid, high-throughput screening methods, comprising: on a biocompatible multiwell substrate, or on multiple biocompatible multiwell substrates, coupling organoids to each other based on their relative spatial arrangement, wherein cells of the organoids have been genetically or chemically perturbed, optionally wherein the substrate is fabricated using the method above; culturing the organoids under conditions that promote development of a target patterned tissue (e.g., complex tissue); and assessing changes in development of, or in one or more characteristic of, the organoids.

Yet further aspects relate to modulating the original shapes of the organoids in addition to their spatial arrangement. The shapes can be parametrized, for instance that of trapezoids, by the size of their parallel sides and the distance between them. In some embodiments, the shapes of the organoids are iteratively optimized using an iterative loop of bioengineering and machine learning.

### BRIEF DESCRIPTION OF THE DRAWINGS

FIG. 1 shows an exemplary machine learning pipeline diagram.

FIG. 2 shows a diagram of random micropattern arrays in a 40 by 40 grid for a range of occupancy probabilities from 1/16 to 1/4 (n=2456 markers from 12 arrays).

FIG. 3 shows an example of a segmented DAPI image corresponding to a random micropattern array.

FIG. 4 shows a graph of a correlation of measured polarization p and major axis length across random array data (r=0.75, n=2373).

FIG. 5 shows a plot of a Pearson correlation of density and gradient features from random array designs from σ=100 μm to σ=2000 μm. Within density and gradient features, a high degree of correlation was observed between features with consecutive values of a. Features with values greater than σ=2000 μm were highly correlated with most features and provided little information.

FIG. 6 shows a graph of sequential forward selection of features with mean RMSE from 5-fold cross-validation. An elbow was found at 2-3 features. Two features were ultimately chosen for simplicity and due to the high correlation of the third chosen feature with one of the first two features.

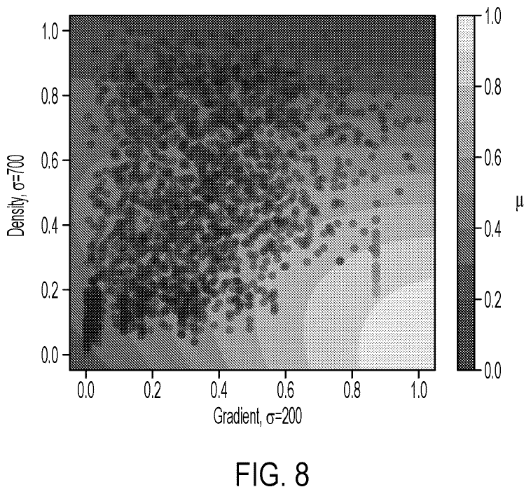

FIG. 7 shows a graph of the minimum predicted polarization across 2-9 markers over 2000 steps of simulated annealing.

FIG. 8 shows a graph of random (black points) and hexagonal arrays with 1.3 mm (dark gray points), 1.6 mm (gray points), and 2 mm (light gray points) inter-hexagon spacing on predicted polarization (μ) landscape. See plots in FIGS. 10G and 10H for measured and predicted polarization values.

FIG. 9 depicts a bioengineering and machine learning framework that optimizes organoid symmetry breaking by tuning their spatial coupling. This framework enabled reproducible generation of axially elongating organoids, each possessing a tailbud and neural tube. Using this framework, it was discovered that an excitable system composed of WNT/FGF signaling drives elongation by inducing a neuromesodermal progenitor-like signaling center. It was also discovered that instabilities in the excitable system are suppressed by secreted WNT inhibitors.

FIGS. 10A-10P show data demonstrating that machine learning-driven optimization of organoid arrangements enables reproducible axial elongation. (FIG. 10A) Left: Micrograph of a human embryo at Carnegie Stage 10 from the Kyoto Collection. Right: Zoomed-in view of posterior neural tube, pseudo-colored to highlight target tissue that includes putative neural tube (anterior, blue) and tailbud (posterior, red). Scale bar 1 mm. (FIG. 10B) Top left: Phase contrast image of 150 μm diameter circular PDMS stamp. Top middle: Phase contrast image of micropatterned epithelial cyst on day 1. Top right: Cyst stained for tight junction marker ZO1. Bottom: Cyst stained for nuclear marker DAPI, and pluripotency factors OCT4 and SOX2. Scale bar 50 μm. (FIG. 10C) Organoid differentiation protocol: day −1, hPSCs seeded in mTeSR Plus (MT) with ROCK inhibitor Y-27632 (RI); day 0, media changed to N2B27 with Matrigel, CHIR99021 (CHIR), LDN193189 (LDN), and A8301; day 2, media changed to N2B27 with CHIR and LDN. Matrigel formed a gel that was maintained throughout 4 days of differentiation. (FIG. 10D) Top: Organoids from low density (left), medium density (middle), and high density (right) random arrays on day 4, stained for SOX1 and CDX2. Scale bar 1 mm. Top right: Zoomed-in view of an unpolarized CDX2+ organoid (bottom) and polarized organoid elongated along the SOX1-CDX2 axis (top). measures the anterior-posterior (A-P) polarization of CDX2 expression (see also Examples 1 and 2). Scale bar 200 μm. (FIG. 10E) Measured polarization (measured) of organoids in (FIG. 10D) plotted on color scale at corresponding micropattern locations in the array. (FIG. 10F) Left: Heat map of organoid density on random arrays evaluated with a Gaussian filter at length scales σ=200 μm (top) and σ=1000 μm (bottom). Right: Visualization of gradient of organoid density using a Sobel filter, evaluated at length scales σ=200 μm (top) and σ=1000 μm (bottom). For gradient calculations, see Methods S1. Scale bar 1 mm. (FIG. 10G) measured of each organoid (n=2373) from 12 random arrays as a function of selected features, gradient of organoid density at σ=200 μm (|∇ρ200|) and density at σ=700 (ρ700). (FIG. 10H) Predicted polarization (pred) for organoids learned using kernel ridge regression with a radial basis function kernel from data in (FIG. 10G) (see also Examples 1 and 2), plotted as a function of |∇ρ200| and ρ700. (FIG. 10I) Initial (left) and final (right) timepoint images of simulated annealing on organoid locations using the function learned in (FIG. 10H) with the number of organoids ranging from 2 (top) to 7 (bottom). Scale bar 300 μm. (FIG. 10J), pred as a function of number of organoids, with a maximum for 6 organoids arranged in a hexagon. (FIG. 10K) maximum, mean, and minimum values of pred for hexagonally arranged organoids as a function of inter-hexagon distance. Arrowhead indicates distance selected for experiments (1.6 mm). (FIG. 10L) Image of computationally optimized hexagonal array. Scale bar 1 mm. (FIG. 10M) Organoids on the optimized hexagonal array on day 4, stained for DAPI, SOX1, and CDX2. All organoids that correctly formed a cyst within a hexagonal unit on day 1 possessed polarized CDX2+ and SOX1+ domains and elongated (n=204). Scale bar 1 mm. (FIG. 10N) Confocal images of organoids from hexagonal array on day 4 stained for laminin subunit gamma-1 (laminin), tight junction protein 1 (ZO1), and mitosis marker phospho-histone H3 (pHH3). Organoids display apicobasal polarity with apical mitosis and a basement membrane. The elongating portion of the organoids are not attached to the coverslip, and are surrounded by laminin-rich extracellular matrix, which is present in the media (see also Examples 1 and 2). Scale bar 100 μm. (FIG. 10O) Results from a mathematical model of symmetry breaking of hexagonally arranged organoids with a small (left) or large (right) center-to-center spacing. Secreted signal gradient is shown in grayscale. At a close spacing, organoids break symmetry coherently and adopt polarized anterior (blue) and posterior (red) fates. Scale bar represents a length of sqrt(4Dτ) (see also Examples 1 and 2). (FIG. 10P) Top: Representative organoids from hexagonal array on consecutive days stained for DAPI, SOX1, and CDX2. Scale bar: 50 μm. Bottom: Violin plot of measured A-P lengths of organoids from a single hexagonal array over time (n=216), with mean (points) and standard deviation (lines), based on Feret diameter (see also Examples 1 and 2). See also FIGS. 1A-1P of Anand G M et al. Cell. 2023 Feb. 2; 186(3): 497-512 (Anand 2023), the content of which is incorporated herein by reference in its entirety.

FIG. 11 depicts a robust human stem cell model of axial morphogenesis with simultaneous neural tube elongation and somite segmentation showing that FGF signaling governs both the dynamics of segmentation clock waves and the somite determination front.

FIG. 12 depicts a protocol outline for organoid culture and bead deposition using microcontact printing.

FIG. 13A shows morphogen-soaked beads in microwells on a PDMS chip capture, generating a gradient across the organoids. FIG. 13B, left panel, shows organoids organized on the chip and the inset (right, top) shows the precise location of the beads deposited near the organoids. FIG. 12A, middle panel, shows the fluorescently labeled beads dispersed on the chip and the inset (right, bottom) shows the precise location of the beads deposited near the organoids. Cells are seeded and folded into 3D and beads are deposited into the wells. Fluorescently-labeled beads are coated in BMP4 and phosphorylated SMAD1/5 (pSMAD) levels are measured (BMP response). FIG. 13C shows signal-coated beads can induce differentiation of adjacent organoids on a chip.

FIG. 14 shows live imaging of OTX2-YFP organoids previously exposed to BMP on micropatterns show increasing lumen size over time, gradual restriction of OTX2 to the roof plate and expression of the roof plate marker LMX1A and dorsal neuroepithelial marker PAX6.

FIG. 15 (Top) shows simultaneous exposure to BMP and SHH beads on opposite sides of the organoids leads to the invagination of the midline (contrast with FIG. 13). OTX2 (live marker) localizes to the midline and also to the ventral side of the organoid over 34 days as the lumen expands. (Bottom) Cleared and immunostained organoid from this protocol at day 40 shows dorsal progenitors (PAX6+), and ventral progenitors (NKX2.2, OLIG2+), and invaginated midline, with the morphology similar to the developing forebrain. Proportions of cell types compared to that in vivo 8-week-old fetal tissue obtained via image analysis of data are shown.

FIG. 16 shows data from experiments relating to the validation of iCRISPRi line and viral construct. Left TOP: image of cells (CFP channel) infected with lentivirus carrying a dual guide and CFP reporter. Middle TOP: dCAS9-RFP imaged in the iRFP channel upon doxycycline induction. Right TOP: Color combine. BOTTOM: Control experiment of CFP virus with guide knocking down surface marker CD55. Contour plot of FACS analysis shows that in cells with CFP have low CD55.

FIG. 17A shows lentivirus carrying two guides against a target gene as well as CFP driven by independent promoters, made in 96 well plates, transferred into 24 well plates, each carrying a hexagonal pattern to infect cells with a 94+/−2% efficiency. FIG. 17B (top) shows cysts arranged in a hexagon, with a central cyst shown over 72 hours, exposed to 10 ng/ml BMP for the first 48 hours, develop an NCADH+ neuroepithelium, which folds and fuses to produce a neural tube inclosing a single lumen, and an surface ectodermal layer (ECADH+) dorsally as the fusion completes. 100% of the peripheral cysts on the hexagon undergo identical morphogenesis. FIG. 17B (bottom) shows organoids in a hexagon, in one row of the 24 well plate, infected with lentiviruses carrying guides against a target gene (named in each subfigure), differentiated for 4 days and stained for NCADH (neural epithelium, green) and ECADH (surface ectoderm, magenta), imaged under an epifluorescence scope, Scale bar 100 um.

FIGS. 18A-18C show multiple geometric arrangements and shapes of organoids. (FIG. 18A) Rectangles are positioned perpendicular to a larger central rectangle, with varying length. (FIG. 18B) Trapezoids are organized in a circle around a center organoid, with varying distance from the center. (FIG. 18C) Organoids are trapezoids surrounded by a U-shaped organoid. Scale bar is 500 microns. Fluorescent staining in FIGS. 17A-17B is OTX2-YFP; fluorescent staining in FIG. 17C is OTX2-YFP (outermost periphery) and LHX2-RFP (throughout tissue structures).

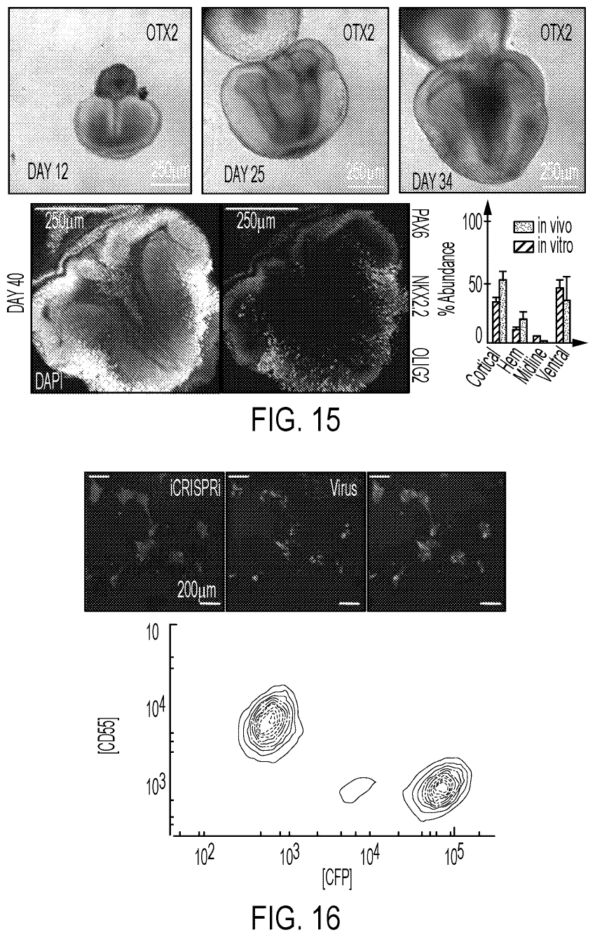

FIG. 19 shows data demonstrating generation of target organoids with neural tube, segmented somites and limb bud using trapezoidal pattern geometry with various arrangements. (FIG. 19A) CAD design of the microprinted patterns. (FIG. 19B) Organoids on the trapezoidal patterns on day 2 of differentiation, stained for TBXT and SOX2. Scale bar, 1 mm. (FIG. 19C) Organoids made of FOXF1-RFP fluorescent reporter cell line on day 2 of differentiation on the optimized patterns. FOXF1. Scale bar, 1 mm. (FIG. 19D) Organoid made of MESP2-RFP fluorescent reporter cell line stained for FOXF1, SOX2 on day 5 of differentiation. Scale bar, 200 um.

FIG. 20 shows eight (8) representative wells of a 24-well plate, 48 hours after viral transduction. The gRNA virus carried a CFP marker to measure transduction efficiency. Transduction was consistently high across wells.

FIGS. 21A and 21B show an analysis of lentiviral transduction efficiency by pixel analysis and nuclear OCT4 knockdown show high efficiency. FIG. 21A, left: Pixel analysis of a control well without transduction shows a cross-well lentiviral contamination rate of 3% cells that show CFP levels above background. FIG. 21A, right: Pixel analysis of a well transduced with anti-OCT4 guide showing transduction efficiency of 87%. FIG. 21B, left: DAPI stain of anti-OCT4 transduced well. FIG. 21B, right: OCT4 stain of anti-OCT4-transduced well showing a knockdown efficiency of 88% of cells.

FIG. 22 shows an excerpt of 5 wells from a 24-well screen against genes known to cause exencephaly in mouse. Left: Diffusion mapping of single cell RNA-seq data of an intermediate folding timepoint of the Karzbrun, et al. (2021) organoid model shows a pseudo-lateral axis with surface ectodermal cells (CDH1), neural plate border (PRDM1), and neural (CDH2) gene expression ordered along a single axis. Right: Similarity scoring between PRDM1 expression and the expression profiles of all other genes shows about 100 putative human neural plate border genes. As neural plate border gives rise to part of the neural fold, we will test the effects of knocking down these genes on neural plate folding.

FIGS. 23A and 23B are diagrams illustrating processing, including processing performed by a trained machine learning (ML) model, to identify spatial arrangements promoting development of stem cell aggregates into a target patterned tissue, according to some embodiments of the technology described herein.

FIG. 24 is a flowchart of an illustrative process 2400 for identifying spatial arrangements promoting development of stem cell aggregates into a target patterned tissue, according to some embodiments of the technology described herein.

FIG. 25 depicts an illustrative implementation of a computer system that may be used in connection with some embodiments of the technology described herein.

### DETAILED DESCRIPTION

Aspects of the disclosure relate to a robust platform for understanding and recapitulating complex, dynamic tissue (e.g., human tissue) development and disease, which can be used to develop and assess gene and drug therapies, for example. The platform described herein. integrates bioengineering and machine learning methods to spatially arrange extracellular matrix protein and stem cells on a biocompatible substrate in such a way as to promote deterministic morphogenesis and organogenesis. This technology enables the exploitation of genetic (e.g., genomic) and/or epigenetic regulation, cellular signaling, pattern formation, mechanical forces, and/or computational analyses to study developmental and disease-causing processes in a reproducible, scalable, rapid, high throughput way.

Understanding mechanisms driving critical events in early human complex tissue development requires systematically studying the effects of penetrant genetic perturbations on patterning, morphogenesis, and physiology. Ethical reasons and differences between humans and model organisms necessitate such studies in robust in vitro models that recapitulate human development. Nonetheless, there is a lack of robust and accurate in vitro models of early human tissue/organ development. While current models using monolayer culture and stem cell aggregates (organoids) recapitulate the cellular composition, they lack spatial organization. They do not possess essential three-dimensional tissue structures for proper organ development.

There is also an inability to perform penetrant genetic perturbations at high throughput to uncover the underlying mechanisms. Current state-of-the-art methods, such as Perturb-Seq, use pooled perturbations of at most thirty-five genes at a time, and these are not penetrant: different cells of the same organoid are perturbed for distinct genes. In contrast, most critical discoveries in developmental biology have been achieved by perturbing the same gene in all the cells of the tissue. Such perturbations are currently impractical at a genome-wide scale in organoids due to high costs. The technology provided herein provides an efficient means to perturb the same gene in all cells of a complex tissue in vitro.

The present disclosure establishes bioengineering methods for printing stem cell cysts, each enclosing a respective lumen. Further, the studies described herein demonstrate how the spatial proximity of these cysts caused them to interact with each other through secreted signals, creating signaling gradients. Statistical learning methods have also been used to determine the spatial arrangement of cysts to achieve reproducible patterning. Using high-dimensional statistical inference to analyze the morphogenesis and patterning of organoids across a diverse set of signaling gradient conditions, the data herein demonstrates that the cysts can be arranged in spatial patterns that establish appropriate signaling gradients, obtaining close to 100% reproducibility in organoid morphogenesis and patterning.

Provided herein, in some aspects, are bioengineering methods for producing a target patterned tissue (e.g., human tissue), the method comprising: coupling organoids to each other based on their relative spatial arrangement; and culturing the organoids under conditions that promote development of the target patterned tissue.

While “stem cells” are referenced throughout this disclosure, progenitor cells (immediate descendants of stem cells) can alternatively be used.

For simplicity herein, the term “organoid” includes one or more cell aggregates (e.g., two-dimensional colony(ies) or a three-dimensional aggregate(s)). Organoids can be derived from and/or include stem cells and/or progenitor cells, e.g., cultured ex vivo. Organoids, in some embodiments, approximate the structure and/or function of a tissue (e.g., human tissue), organ, or organ system. A “cell aggregate” includes one or more cluster(s)/group(s) of cells that have come together to form a multicellular structure. Thus, the terms “cell aggregate” and “multicellular structure” can be used interchangeably herein.

A “cyst,” such as an epithelial cyst, includes a layer of cells that enclose a single lumen. In some embodiments, cysts of the disclosure develop into organoids (e.g., three-dimensional organoids) under suitable culture conditions.

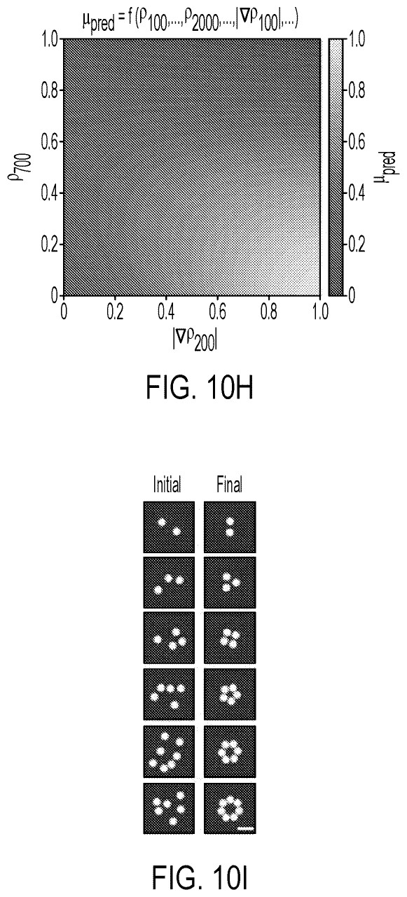

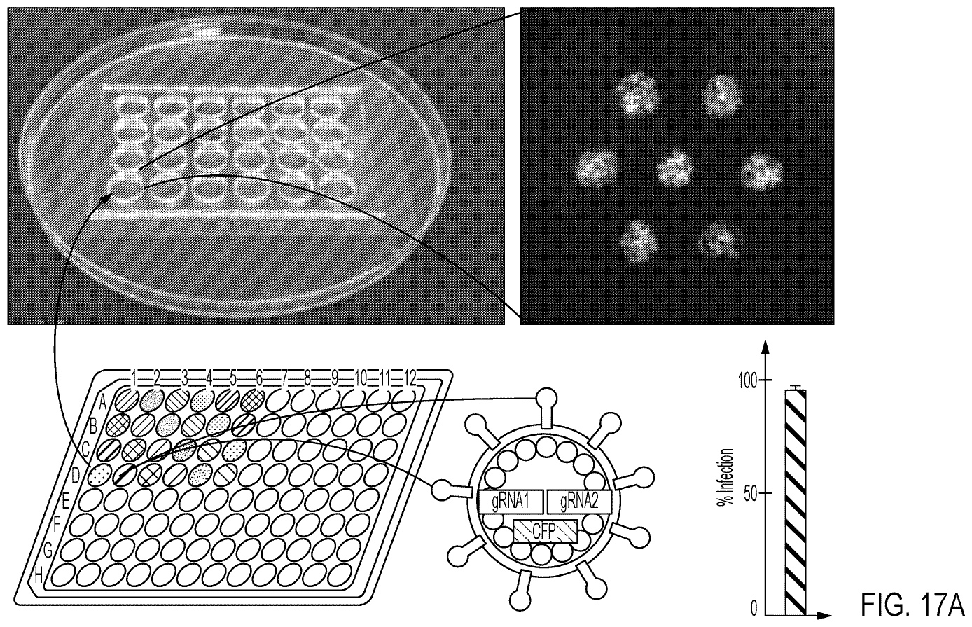

A “spatial arrangement” with respect to organoids, for example, describes the position of organoids on a substrate relative to one another (relative spatial arrangement). For example, a spatial arrangement may be hexagonal—thus, the organoids are arranged on a substrate to form a hexagonal shape (hexagon) (see, e.g., FIG. 10O). Similarly, a “spatial arrangement” with respect to cells describes the position of cells on a substrate within a defined area, for example, a circle or polygonal shape (e.g., trapezoid) (see, e.g., FIG. 19). Stem cells deposited on a substrate in the shape of a circle or polygonal shape develop into cysts, which develop into organoids under suitable conditions, as described herein. Spatial arrangements are typically determined using a machine learning model provided herein.

Organoids can be considered “coupled” to each other if the organoids can signal to each other to drive development processes such as elongation, proliferation, and/or migration.

A “target patterned tissue” includes a structure formed from developing organoids that includes one or more characteristics of a complex tissue of interest, such as size, shape, cell diversity, cells density, functional properties, etc.). In some embodiments, a target patterned tissue is a complex tissue, such as a complex human or other animal tissue.

### Generating Spatial Arrangements of Stem Cell Aggregates

The inventors have recognized and appreciated that stochastic optimization and machine learning (ML) techniques can provide a platform for accurately and reproducibly predicting, in silico, stem cell aggregate arrangements that, when cultured, mature into tissue have desired characteristics. Accordingly, the inventors have developed techniques for generating optimized spatial arrangements of stem cell aggregates, wells, and/or beads for the culturing of stem cell aggregates into target patterned tissue. The techniques include training an ML model to predict patterning metrics given feature vectors calculated based on a given spatial arrangement of stem cell aggregates and, optionally, beads. The techniques further include using a process in which stochastic optimization is used to explore, computationally, the space of possible spatial stem cell arrangements by evaluating potential spatial stem cell arrangements through patterning metrics for those arrangements obtained using the trained ML model. As such, stochastic optimization may be used to optimize the patterning metrics (and therefore the associated spatial arrangements and tissue that would result from culturing such optimized spatial arrangements) by iteratively altering spatial arrangements and/or associated feature vectors and evaluating. The optimized spatial arrangements (e.g., spatial arrangements having sufficiently high patterning metric(s) value(s)), discovered using the stochastic optimization procedure, may then be used to generate new configurations of stem cell aggregates and/or beads to test experimentally. Data obtained through experimental testing of the new configurations may be, in turn, used to generate more training data to improve the trained ML model.

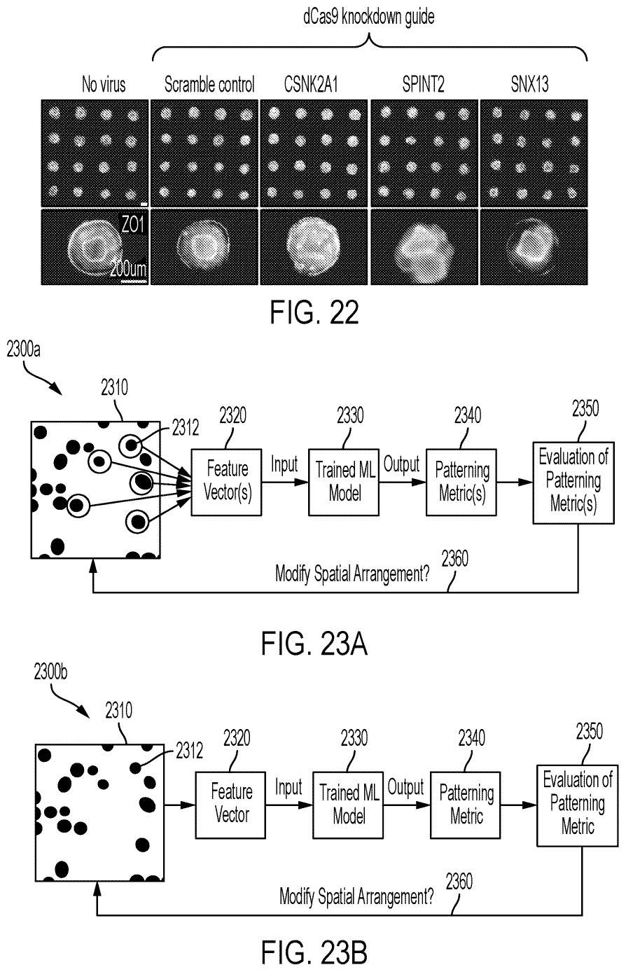

FIGS. 23A and 23B are diagrams illustrating processing pipelines 2300a and 2300b, according to some embodiments of the technology described herein. The processing pipelines 2300a, 2300b include processing performed by a trained machine learning (ML) model and are configured to identify spatial arrangements promoting development of stem cell aggregates into a target patterned tissue. The processing described with reference to processing pipelines 2300a and 2300b may be performed using any suitable computing device or devices, as aspects of the technology described herein are not limited in this respect. For example, the processing may be performed by a computing device that is part of or attached to or communicatively coupled to an optical microscopy device. Additionally or alternatively, the processing may be performed by a standalone computing device (e.g., a laptop, desktop, tablet, mobile device, or other suitable computing device). In some embodiments, portions of the processing may be performed by a single computing device or by multiple computing devices (e.g., computing devices that are located remotely from one another and communicatively coupled over a network connection, cloud computing system, or other communication network).

In some embodiments, the processing pipelines 2300a and 2300b receive as input a spatial arrangement 2310. The spatial arrangement 2310 may be, for example, an image of a spatial arrangement of stem cell aggregates 2312. The spatial arrangement 2310 may include markers indicating spatial positions of the stem cell aggregates 2312. The markers may be shaped to indicate the relative sizes of the stem cell aggregates and/or to indicate morphological features of the stem cell aggregates 2312. Additionally, in some embodiments, the spatial arrangement 2310 may include one or more additional markers representing the respective locations of one or more beads (e.g., as might be placed in wells at those locations to influence the target patterned tissue cultured from the arrangement of stem cell aggregates).

In some embodiments, the spatial arrangement 2310 may be an image generated using computational methods. For example, the spatial arrangement 2310 may be generated by randomly placing one or more markers representing stem cell aggregate locations and/or bead locations (e.g., for one or more bead types) in a two-dimensional array. In some embodiments, the spatial arrangement 2310 may be generated by sequential sampling of polarization features, whereby individual markers may be placed within the two-dimensional array at locations maximizing one or more patterning metrics (e.g., polarization). In some embodiments, the synthetic image may be generated based on one or more experimental images. For example, segmented images of stem cell aggregates and/or beads may be arranged at locations within a two-dimensional array to generate the spatial arrangement 2310. Additional aspects of generating synthetic images are described in Section 1.5.1 of Example 1 herein.

In some embodiments, after obtaining a spatial arrangement 2310, the processing pipelines 2300a and/or 2300b may proceed to generate one or more feature vectors 2320 using the spatial arrangement 2310. As seen in the example of FIG. 23A, the processing pipeline 2300a may perform processing for each stem cell aggregate 2312 to generate a respective one or more feature vectors. For example, a single respective feature vector 2320 may be generated for each stem cell aggregate 2312 in the spatial arrangement 2310. Alternatively, a single feature vector 2320 describing the whole of the spatial arrangement 2310 may be generated based on the processing of each stem cell aggregate 2312. In contrast, and as seen in the example of FIG. 23B, the processing pipeline 2300b may perform processing on the stem cell aggregates 2312 of the entire spatial arrangement 2310 such that a single feature vector 2320 is generated for the spatial arrangement 2310. Such a feature vector could include features calculated for each of the stem cell aggregates in the spatial arrangement and/or one or more other types of features, examples of which are provided herein.

In some embodiments, a feature vector (or one of feature vectors) 2320 may be a vector including values indicative of features of the spatial arrangement 2310. In some embodiments, for example, the values of the feature vector may include one or more densities for one or more stem cell aggregates in the spatial arrangement 2310 and/or one or more density gradients for one or more stem cell aggregates in the spatial arrangement 2310. In some embodiments, to generate density and/or density gradient values, respective length scales for computation of the density and/or density gradient values may first be selected. To determine relevant length scales for calculating the density and/or density gradient values, Gaussian smoothing, with varying σ values, may be applied to the spatial arrangement 2310. Density and/or density gradient values may then be calculated for each of the smoothed spatial arrangements for the varying σ values, as described in Section 1.4.2 of Example 1 herein. Thereafter, suitable respective length scales, σ, may be selected for calculating the density and/or the density gradient values by evaluating correlation (e.g., using Pearson correlation) between density and/or density gradient features and using sequential forward selection techniques as described in Section 1.4.2 of Example 1 herein.

In some embodiments, the respective length scales may depend on sizes of the stem cell aggregates. For example, the stem cell aggregates may be 150 microns in diameter such that the respective length scales for calculating the density and/or density gradient values may be selected from values in a range from 50 to 2000 microns. In some embodiments, the respective length scales for calculating the density and/or density gradient values may be different length scales. In some embodiments, the length scale for calculating the density values may be in a range from 400 to 1200 microns, in a range from 500 to 1000 microns, in a range from 600 to 800 microns, or any suitable range or value within those ranges. For example, the length scale for calculating the density values may be 700 microns. In some embodiments, the length scale for calculating the density gradient values may be in a range from 50 to 400 microns, in a range from 50 to 300 microns, in a range from 75 to 125 microns, or any suitable range or value within those ranges. For example, the length scale for calculating the density gradient values may be 200 microns.

In some embodiments, the one or more feature vectors 2320 may include values of one or more higher-order density derivatives for one or more stem cell aggregates in the spatial arrangement 2310. For example, higher-order density derivatives may be calculated for suitable length scales using higher-order derivative analogues of the equation describing the density gradient in Section 1.4.2 of Example 1 herein.

In some embodiments, the one or more feature vectors 2320 may include values representing the image of the spatial arrangement 2310 and/or an image of a portion of the spatial arrangement 2310. For example, the portion of the spatial arrangement 2310 may be a segmented and/or cropped portion of the spatial arrangement 2310 showing a subset of stem cell aggregate locations. In some embodiments, the image of the spatial arrangement 2310 and/or the image of the portion of the spatial arrangement 2310 may be provided directly to the trained ML model 2330.

In some embodiments, the one or more feature vectors 2320 may include values of one or more Fourier transform components. The one or more Fourier transform components may be calculated using the image of the spatial arrangement 2310 and/or an image of a portion of the spatial arrangement 2310 (e.g., a segmented or cropped portion of the spatial arrangement 2310 showing a subset of stem cell aggregate locations). The one or more Fourier transform components may be computed using a two-dimensional Fourier transform of the image of the spatial arrangement 2310 and/or the image of the portion of the spatial arrangement 2310. The feature vector may include the resulting Fourier coefficients (which may be complex-valued), their magnitudes, and/or any other values derived from the Fourier coefficients. Other transformations may be used as well, for example, a short-time Fourier transform or a wavelet transform, with one or more components of the so transformed image (e.g., one or more STFT coefficients, wavelet coefficients, a function thereof) used as part of the feature vector.

In some embodiments, the one or more feature vectors 2320 may include values of one or more bead positions within the spatial arrangement 2310, when the spatial arrangement 2310 includes at least one bead (e.g., heparin-agarose beads (Sigma)). For example, the values of the one or more bead positions may correspond with locations of microwells that, when formed in a suitable substrate (e.g., polymer substrate such as PDMS), are configured to hold the beads at the desired locations.

In some embodiments, the one or more feature vectors 2320 may include values representative of one or more bead types within the spatial arrangement 2310, when the spatial arrangement includes at least one bead (e.g., magnetic, polymer, silica, glass, ceramic, or hydrogel). For example, the values may represent beads configured to secrete different signals or beads coated with different morphogen coatings. Alternatively or additionally, the values may represent beads having different sizes and/or shapes.

In some embodiments, the one or more feature vectors 2320 may include values representative of one or more shapes of one or more stem cell aggregates in the spatial arrangement 2310. For example, the values may be representative of one or more shape parameters defining an aspect of the stem cell aggregate shape. As one example, in the case of a trapezoidal stem cell aggregate, the values parameterizing the stem cell aggregate shape may include one or more of lengths of the sides of the trapezoid and/or a height of the trapezoid. As an additional example, the values may include, for any polygon: lengths of sides of the polygon, angles between sides of the polygon, an area of the polygon, or any other geometric feature of the polygon. For a circle, the values may include, for example, the radius, diameter, circumference, area, or any other geometric feature of the circle. For an ellipsoid, the values may include, for example, lengths of the semi-major and semi-minor axes, the eccentricity, the circumference, the area, or any other geometric feature of the ellipsoid. Alternatively or additionally, the values may include values representative of one or more stem cell aggregate shape or morphological types.

In some embodiments, the one or more feature vectors 2320 may be provided as input to a trained ML model 2330. In some embodiments, the trained ML model 2330 may be a regression model. For example, the trained ML model 2330 may be a linear regression model or a non-linear regression model. In some embodiments, the trained ML model 2330 may be a non-linear regression model that is a kernel regression model (e.g., a kernel ridge regression model). The kernel regression model may use a linear or radial basis function (RBF) kernel function and/or any other suitable type of kernel function. In some embodiments, the trained ML model 2330 may be a support vector machine (SVM) regression model. The SVM regression model may use a linear or RBF kernel function, and/or any other suitable type of kernel function. In some embodiments, the trained ML model 2330 may be a random forest regression model. In some embodiments, the trained ML model 2330 may be a decision tree regression model.

In some embodiments, the trained ML model 2330 may be a neural network model. The neural network model may have an architecture including a single hidden layer or multiple hidden layers (e.g., the neural network model may be a deep neural network model). In some embodiments, the neural network model may have one or more convolutional layers and/or may have a convolutional neural network architecture. In some embodiments, the neural network model may have one or more fully connected layers, one or more convolutional layers, one or more batch normalization layers, and/or any other layer(s) suitable for use as part of a neural network regression model, as aspects of the technology described herein are not limited in this respect.

In some embodiments, the trained ML model 2330 may be trained to generate, as output, one or more patterning metrics 2340 using the one or more feature vectors 2320. As seen in the example of FIG. 23A, the trained ML model 2330 may be arranged to generate one or more patterning metrics 2340 corresponding to each of the one or more feature vectors 2320 and to each of the associated stem cell aggregates 2312. In contrast, and as seen in the example of FIG. 23B, the trained ML model 2330 may be arranged to generate a single patterning metric or set of patterning metrics corresponding to the single feature vector 2320 and the associated spatial arrangement 2310. Examples of patterning metrics are provided herein.

In some embodiments, the one or more patterning metrics 2340 comprises one or more values indicative of a predicted expression for each of one or more biomarkers associated with patterned tissue to be generated using stem cell aggregates having the spatial arrangement 2310. For example, the one or more patterning metrics 2340 may include one or more values indicative of one or more of: a polarization of predicted expression of a biomarker, a dipole moment of the predicted expression of the biomarker, a multipole moment of the predicted expression of the biomarker, a spatiotemporal pattern of the polarization, the dipole moment, and/or the multipole moment of the predicted expression of the biomarker, predicted expression and/or predicted patterns of expression of cell signals (e.g., secreted biomarker(s)), and/or morphology of a stem cell aggregate and/or tissue to be generated using stem cell aggregates arranged in the spatial arrangement 2310.

As one example, in some embodiments, the one or more patterning metrics 2340 may include values indicative of a polarization of the predicted expression of a biomarker by cultured stem cell aggregates. The polarization may be a measure of symmetry breaking (e.g., elongation) of the predicted expression of the biomarker. For example, expression of the biomarker may be measured as expression of a gene by the cultured stem cell aggregates. As one example, the gene may be CDX2, as described in Example 2 herein. As another example, the biomarker may be identified by expression of cell signals (e.g., secreted biomarkers). In some embodiments, the trained ML model 2330 may be trained, using measured polarization values and measured feature vectors, to generate estimated polarization values according to Section 1.4.1 of Example 1 herein.

In some embodiments, after obtaining the one or more patterning metrics 2340 using the trained ML model 2330, the processing pipelines 2300a and/or 2300b may perform an evaluation 2350 of the obtained one or more patterning metrics 2340. For example, the values of the one or more patterning metrics 2340 may be compared to values of a previously computed patterning metric (e.g., as obtained from a prior spatial arrangement using the trained ML model 2330). The comparison may be used to determine whether the values of the one or more patterning metrics 2340 are improved relative to the values of the prior patterning metric (e.g., whether the values of the one or more patterning metrics 2340 are greater than the values of the prior patterning metric). Alternatively or additionally, the values of the one or more patterning metrics 2340 may be compared to previously determined optimized values of patterning metrics. In such embodiments, the evaluation 2350 may include determining if the values of the one or more patterning metrics 2340 are closer to the values of the determined optimized values than values of a prior patterning metric associated with a prior spatial arrangement.

In some embodiments, the evaluation 2350 may be part of a stochastic optimization technique such that the spatial arrangement 2310 may be accepted as a new basis for optimization if, during the evaluation 2350, the one or more computing devices determine that the values of the one or more patterning metrics 2340 are improved relative to values of a prior patterning metric. Additionally, in some embodiments, the spatial arrangement 2310 may be accepted with some probability even if the values of the one or more patterning metrics 2340 are not improved relative to values of a prior patterning metric in order to allow for exploration of a larger parameter space during the stochastic optimization.

In some embodiments, the stochastic optimization technique may be simulated annealing. In some embodiments, simulated annealing may be used with acceptance defined as a function of output of the trained ML model 2330 (e.g., as a function of the one or more patterning metrics 2340). As another example, the stochastic optimization technique may be an iterative descent algorithm (e.g., a stochastic gradient descent algorithm) with a step direction being a function of output of the trained ML model 2330 (e.g., as a function of the patterning metric(s) 2340).

In some embodiments, if the spatial arrangement 2310 is accepted after the evaluation 2350, the one or more computing devices may modify the spatial arrangement 2310 to generate a new spatial arrangement to provide to the processing pipelines 2300a and/or 2300b as a part of the stochastic optimization technique. In some embodiments, the modification 2360 may include perturbing a random stem cell aggregate and/or bead location in a random direction, as described in Section 1.5.2 of Example 1 herein. The perturbed spatial arrangement may be selected for use in the stochastic optimization process if one or more of the one or more patterning metrics, as determined by the trained ML model 2330, are improved relative to the one or more patterning metrics 2340 associated with the spatial arrangement 2310.

In some embodiments, the processing pipelines 2300a and/or 2300b may be repeated iteratively to optimize one or more characteristics of the spatial arrangement. For example, the relative stem cell aggregate positions, stem cell shapes, and/or locations or types of beads may be optimized using the processing pipelines 2300a and/or 2300b. The optimization may be performed sequentially (e.g., optimizing the stem cell aggregate positions, shapes, and/or locations or types of beads one by one) or jointly. The optimization may also be performed for a set number of steps (e.g., tens, hundreds, thousands, tens of thousands of steps, etc.) or until a specified stopping criterion is met (e.g., when the one or more patterning metrics 2340 are within a specified distance of specified patterning metric values, when iterative changes to spatial arrangements do not yield at least a threshold difference between respective patterning metrics indicating convergence to a local or global optimum). In this manner, an optimized spatial arrangement of stem cell aggregates may be generated for further experimental testing using the processing pipelines 2300a and/or 2300b. In turn, data obtained during experimental testing may be used to further train the trained ML model 2330, and the overall pipelines shown in FIGS. 23A and 23B may be repeated.

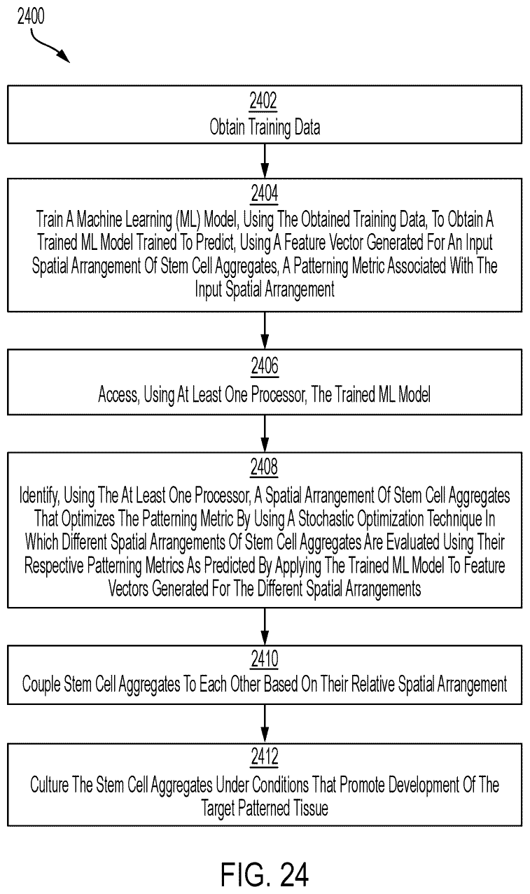

FIG. 24 is a flowchart of an illustrative process 2400 for identifying spatial arrangements promoting development of stem cell aggregates into a target patterned tissue, in accordance with some embodiments of the technology described herein. Process 2400 may be performed by any suitable computing device or devices, as aspects of the technology described herein are not limited in this respect. In some embodiments, process 2400 may be implemented by a computing device that is part of or attached to or is communicatively coupled to an optical microscopy device. Alternatively, process 2400 may be implemented by a standalone computing device (e.g., a laptop, desktop, tablet, mobile device, or other suitable computing device). In some embodiments, portions of process 2400 may be performed by computing devices that are located remotely from one another (e.g., computing devices that are communicatively coupled over a network connection, cloud computing system, or other communication network).

In some embodiments, process 2400 may begin at act 2402, in which training data is obtained by the one or more computing devices implementing the process 2400. Obtaining the training data may include, for example, accessing the training data from one or more computer readable memories (e.g., local or remote computer memories) or receiving the training data from another computing device. The training data may include, for each particular spatial arrangement of multiple spatial arrangements of stem cell aggregates and/or beads, at least one measured value indicative of a patterning metric for the particular spatial arrangement and a feature vector generated from information specifying the particular spatial arrangement. As one non-limiting example, the training data may include data pairs of feature vectors including measured density and density gradient values and patterning metrics including polarization values of CDX2 intensity, as described in Section 1.4 of Example 1 herein.

In some embodiments, obtaining the training data may include generating the training data. Generating the training data may include first generating multiple random patterns (e.g., random patterns corresponding to random arrangements of stem cell aggregates and/or beads). One method to generate the multiple random patterns includes assigning positions within a two-dimensional array with one of a set of probabilities of being occupied by a stem cell aggregate and/or a bead. For example, and as described in Section 1.2 of Example 1, the positions may be assigned one of a probability of being occupied that is in a range from 0.0625 to 0.25.

After generating the multiple random patterns, stem cell aggregates and/or beads may be patterned in micropattern arrays printed according to the multiple random patterns. The experimental arrangement of stem cell aggregates and/or beads may then be cultured on one or multiple two-dimensional substrates and/or in a three-dimensional matrix, as described herein. Thereafter, the cultured stem cell aggregates may be stained (e.g., antibody-stained), and stained images of the experimental arrangement may be obtained. Alternatively or additionally, in situ probe-hybridized RNA and/or live fluorescent reporter images of the cultured stem cell aggregates may be obtained for each of one or more biomarkers. The obtained images may be further processed prior to the generation of the training data, as described in Section 1.3 of Example 1.

In some embodiments, the training data may be generated by generating pairs of feature vectors and patterning metrics using the obtained images. The feature vectors may be generated for each of the random patterns by measuring and/or calculating aspects of the obtained images to obtain values of the feature vectors. For example, density values, density gradient values, one or more higher-order derivatives of the density values, one or more Fourier transform components, bead locations, bead identities, and/or one or more shape parameters may be calculated for each of the obtained images to generate the feature vectors. In some embodiments, corresponding patterning metric values may also be generated using the obtained images. For example, generating the patterning metric values may include determining a polarization of the expression of a biomarker for given stem cell aggregates in the obtained images as described in Section 1.4.1 of Example 1. Alternatively or additionally, generating the patterning metric values may include determining one or more of: a dipole moment the expression of a biomarker, a multipole moment of the expression of the biomarker, a spatiotemporal pattern of the expression, polarization, dipole moment, and/or multipole moment of the expression of the biomarker, an expression and/or pattern of expression of cell signals (e.g., secreted biomarker(s)), and/or morphology of a stem cell aggregate and/or tissue to be generated using the stem cell aggregates in the obtained images.

After obtaining the training data pairs in act 2402, the process 2400 may proceed to act 2404, in which an ML model is trained using the obtained training data to obtain a trained ML model. In some embodiments, the trained ML model is trained to predict, using a feature vector generated for an input spatial arrangement of the stem cell aggregates, a patterning metric associated with the input spatial arrangement.

In some embodiments, the ML model may be trained by estimating values of parameters of the ML model using the obtained training data. As one non-limiting example, the ML model may be a kernel ridge regression model using a linear or RBF kernel function. The kernel ridge regression model may be trained to generate the trained ML model using the grid search algorithm to estimate the values of the regularization parameter, λ, and length scale parameter, s, as described in Section 1.4.4 of Example 1. Alternatively or additionally, least squares, nonlinear least squares, regularized linear least squares, or regularized nonlinear least squares algorithms may be used to estimate λ and s of the kernel ridge regression model.

As another example, in some embodiments, the ML model may be a neural network model. The neural network model may have an architecture including a single non-linear layer or multiple non-linear layers (e.g., the neural network model may be a deep neural network model). In some embodiments, the neural network model may have one or more convolutional layers and/or may have a convolutional neural network architecture. In some embodiments, the neural network model may have one or more fully connected layers, one or more convolutional layers, one or more batch normalization layers, and/or any other layer(s) suitable for use as part of a neural network regression model.

In some embodiments, the neural network regression model may be trained using any suitable neural network optimization software. The optimization software may be configured to perform neural network training by gradient descent, stochastic gradient descent, or in any other suitable way. In some embodiments, the Adam optimizer (Kingma, D. and Ba, J. (2015) Adam: A Method for Stochastic Optimization. Proceedings of the 3rd International Conference on Learning Representations (ICLR 2015)) may be used to train the neural network regression model.

It should be appreciated that the above examples of ML models are non-limiting, and that in some embodiments the ML model may alternatively or additionally be an SVM regression model, a random forest regression model, and/or a decision tree regression model trained using suitable training techniques, as aspects of the technology described herein are not limited in this respect.

After obtaining the trained ML model in act 2404, in some embodiments the process 2400 may proceed to act 2406, in which the trained ML model may be accessed using at least one computer hardware processor. In some embodiments, the at least one computer hardware processor configured to access the trained ML model may be different than the computer hardware processor(s) used to generate the training data and/or to generate the trained ML model.

In some embodiments, the at least one computer hardware processor may access the trained ML model from one or more computer readable memories storing the trained ML model. For example, the at least one computer hardware processor may access the trained ML model from local computer readable memory. Alternatively or additionally, the at least one computer hardware processor may access the trained ML model from one or more remote computer readable memories (e.g., over a communication network, using a cloud computing environment, etc.).

After act 2406, the process 2400 may proceed to act 2408, in which an optimized spatial arrangement of stem cell aggregates may be identified. The optimized spatial arrangement of stem cell aggregates may be identified using a stochastic optimization technique and the trained ML model. The stochastic optimization technique may be performed using processing pipelines 2300a and/or 2300b. The stochastic optimization technique may include iteratively evaluating different spatial arrangements of stem cell aggregates and/or beads. Non-limiting examples of the stochastic optimization technique may be simulated annealing, stochastic gradient descent, stochastic hill climbing, iterated local search, evolution and/or genetic algorithms, or swarm optimization.

In some embodiments, to iteratively evaluate the different spatial arrangements, feature vectors may be generated for the different spatial arrangements. The feature vectors may include values indicative of features of a given spatial arrangement. For example, the feature vectors may include values indicative of densities of, density gradients of, and/or higher-order derivatives of densities of one or more stem cell aggregates in a given spatial arrangement. The feature vectors may additionally or alternatively include one or more of values representing an image, or portion of an image, of a given spatial arrangement or one or more Fourier (or other type of) transform components of the image, or portion of an image, of a given spatial arrangement. The feature vectors may additionally or alternatively include values of one or more bead positions and/or bead types for spatial arrangements including beads. The feature vectors may additionally or alternatively include values representative of one or more shapes of one or more stem cell aggregates in a given spatial arrangement.

In some embodiments, the generated feature vectors may then be provided to the trained ML model to generate one or more patterning metrics for a given spatial arrangement. The one or more patterning metrics may include one or more values indicative of a predicted expression for each of one or more biomarkers associated with patterned tissue to be generated using stem cell aggregates having been arranged according to the given spatial arrangement. For example, the one or more patterning metrics may include one or more of: a polarization of predicted expression of a biomarker, a dipole moment of the predicted expression of the biomarker; a multipole moment of the predicted expression of the biomarker, a spatiotemporal pattern of the polarization, the dipole moment, and/or the multipole moment of the predicted expression of the biomarker; predicted expression and/or predicted patterns of expression of cell signals (e.g., secreted biomarker(s)), and/or morphology of a stem cell aggregate and/or tissue to be generated using stem cell aggregates arranged in the given spatial arrangement.

In some embodiments, the generated patterning metrics may then be evaluated to determine a next step of the stochastic optimization process. For example, the values of the one or more patterning metrics may be compared to values of a prior patterning metric (e.g., as obtained from a prior spatial arrangement using the trained ML model). The comparison may determine whether the values of the one or more patterning metrics are improved relative to the values of the prior patterning metric (e.g., whether the values of the one or more patterning metrics are greater than the values of the prior patterning metric). Alternatively or additionally, the values of the one or more patterning metrics may be compared to previously determined optimized values of patterning metrics. In such embodiments, the evaluation may include determining if the values of the one or more patterning metrics are closer to the values of the determined optimized values than values of a prior patterning metric associated with a prior spatial arrangement (e.g., by evaluating a distance between the one or more patterning metric values and the determined optimized values).

In some embodiments, the steps of act 2408 may be repeated iteratively to optimize one or more characteristics of the spatial arrangement. For example, the relative stem cell aggregate positions, stem cell shapes, and/or locations or types of beads may be optimized as described above. The optimization may be performed sequentially (e.g., optimizing the stem cell aggregate positions, shapes, and/or locations or types of beads one by one) or jointly. The optimization may also be performed for a set number of steps (e.g., tens, hundreds, thousands, tens of thousands of steps, etc.) or until a specified stopping criterion is met (e.g., when the one or more patterning metrics are within a specified distance of specified patterning metric values, when iterative changes to spatial arrangements do not yield at least a threshold difference between respective patterning metrics indicating convergence to a local or global optimum). In this manner, an optimized spatial arrangement of stem cell aggregates may be generated for further experimental testing.

After identifying a spatial arrangement in act 2408, the process 2400 may proceed to act 2410, in which stem cell aggregates and/or beads may be coupled to each other based on their relative spatial arrangement. The stem cell aggregates and/or beads may be coupled to each other to generate the spatial arrangement identified in act 2408. In some embodiments, coupling the stem cell aggregates and/or beads includes micropatterning stem cells on a biocompatible substrate, as described herein, according to the identified spatial arrangement.

After act 2410, the process 2400 may proceed to act 2412, in which the coupled stem cell aggregates may be cultured under conditions that promote development of a target patterned tissue. In some embodiments, culturing the micropatterned stem cells on the biocompatible substrate includes forming cysts, each enclosing a respective lumen, and exposing the cysts to conditions that confer identity of the stem cell aggregates.

In some embodiments, the cultured stem cell aggregates obtained from act 2412 may be used to generate additional training data to improve the predictive capability of the ML model. For example, the cultured stem cell aggregates may be antibody-stained and used to obtain stained images. The stained images may then be used to generate a new training data pair, as described in connection with act 2402, which may be used to retrain the trained ML model, or, alternatively, to train an untrained ML model. Thereafter, the stochastic optimization technique of act 2408 may be implemented using the improved trained ML model obtained using the new training data pair. The training of the ML model may be performed iteratively to continually incorporate additional training data and to improve the predictive capabilities of the resulting trained ML model.

The relative spatial arrangement of organoids (e.g., at least 2, 5, 10, 15, 20, or more) with respect to one another can be determined using any one of the machine learning (ML) models described herein. For example, the relative spatial arrangement of organoids for use in a bioengineering model can be determined by (a) accessing a trained ML model trained to predict, from multi-scale geometric features determined for a particular organoid in a spatial arrangement of organoids, a characteristic of the particular organoid; and (b) identifying the at least one spatial arrangement of organoids that promotes development of the organoids into the target patterned tissue by using a stochastic optimization technique in which spatial arrangements of organoids are evaluated using the trained ML model.

Coupling organoids to each other (e.g., coupling an organoid to one or more additional organoids) comprises depositing stem cells on a biocompatible substrate in a predetermined spatial arrangement such that they develop into cysts, which then develop into organoids that are capable of signaling to each other and differentiating into a target patterned tissue (e.g., complex tissue). In some embodiments, the predetermined spatial arrangement is fabricated on a substrate by micropatterning extracellular matrix protein in that predetermined spatial arrangement.

A predetermined spatial arrangement, in some embodiments, promotes development of stem cells into cysts, which develop into organoids, which develop into a target patterned tissue. A predetermined spatial arrangement can be a geometric pattern, for example. A geometric pattern can be a triangular, quadrilateral, pentagonal, hexagonal, heptagonal, octagonal, nonagonal, or decagonal pattern. In some embodiments, a geometric pattern includes squares, circles, rectangles, ovals, triangles, pentagons, hexagons, octagons, or diamonds. In some embodiments, predetermined spatial arrangement is hexagonal.

Micropatterning involves the arrangement of stem cells on a micro scale to control the geometry of adhesion of the cells. In some embodiments, micropatterns can be designed to optimize the development and growth of the stem cells into organoids, and ultimately into a target patterned tissue. Herein, the micropatterns (of extracellular matrix protein) are repeating such that a single substrate can include multiple micropatterns (e.g., multiple hexagonal shapes), each micropattern including multiple developing cysts/organoids (e.g., at least 2, 3, 4, 5, 6, or more). In some embodiments, each micropatterns is designed to include about 2 to about 10 cysts/organoids. The design of a micropattern may take into consideration a variety of factors including at least the size of the organoids, spacing of the organoids, density of the organoids, density gradient of the organoids, polarization of the organoids, and type of organoid. In some embodiments, an organoid is coupled to one or more other organoids such that all the organoids influence the patterning and morphogenesis of each other based on their relative spatial arrangement. The term “cyst/organoid” means “cyst and/or organoid”.

A distance between the center of any two (or more) cysts/organoids on a substrate (e.g., a biocompatible substrate) can be about 100 microns to about 1000 microns. In some embodiments, a distance between the center of any two (or more) cysts/organoids on a substrate is 50-1000 μm, 100-1000 μm, 150-1000 μm, 200-800 μm, 250-800 μm, 300-800 μm, 350-1000 μm, 400-800 μm, 450-1000 μm, 500-800 μm, 550-1000 μm, 600-800 μm, 650-1000 μm, 700-800 μm, 750-1000 μm, or 800-1000 μm.

A diameter of each of the cysts/organoids (e.g., organoids on a substrate) can be about 100 microns to about 1000 microns. In some embodiments, a diameter of each of the cysts/organoids is 50-1000 μm, 100-1000 μm, 150-1000 μm, 200-800 μm, 250-800 μm, 300-800 μm, 350-1000 μm, 400-800 μm, 450-1000 μm, 500-800 μm, 550-1000 μm, 600-800 μm, 650-1000 nm, 700-800 nm, 750-1000 nm, or 800-1000 nm.

An area occupied by each of the cysts/organoids can be about 5,000 μm² to about 500,000 μm². In some embodiments, an area is about 5,000-250,000 μm², 5,000-10,0000 μm², 5,000-10,000 μm², 10,000-50,000 μm², or 25,000-50,000 μm².

A geometric pattern of extracellular matrix protein may have an internal diameter (or length or width) of about 50 μm, 100 μm, 150 μm, 200 μm, 250 μm, 300 μm, 350 μm, 400 μm, 450 μm, 500 μm, 600 μm, 650 μm, 700 μm, 750 μm, or 800 μm. In some embodiments, a geometric pattern has an internal diameter of 50-1000 μm, 100-1000 μm, 150-1000 μm, 200-800 μm, 250-800 μm, 300-800 μm, 350-1000 μm, 400-800 μm, 450-1000 μm, 500-800 μm, 550-1000 μm, 600-800 μm, 650-1000 μm, 700-800 μm, 750-1000 μm, or 800-1000 μm. A geometric pattern may have a length along one axis of at least 150 μM, 200 μM, 250 μM, 300 μM, 350 μM, 400 μM, 450 μM, 500 μM, 550 μM, 600 μM, 650 μM, 700 μM, 750 μM, 800 μM, 850 μM, 900 μM, 950 μM, or 1000 μM. In some embodiment, a geometric pattern has a length along one axis of 50-1000 μm, 100-1000 μm, 150-1000 μm, 200-800 μm, 250-800 μm, 300-800 μm, 350-1000 μm, 400-800 μm, 450-1000 μm, 500-800 μm, 550-1000 μm, 600-800 μm, 650-1000 μm, 700-800 μm, 750-1000 μm, or 800-1000 μm.

In some embodiments, a rectangular pattern (e.g., square pattern) of extracellular matrix protein involves the growth of four organoids per micropattern. In some embodiments, the cysts/organoids have a diameter of about 100 μm to about 500 μm (e.g., about 150 μm). In some embodiments, the micropattern has an overall 200×200 μm size, e.g., the cysts/organoids are spaced at a distance of about 200 μm from each other. In some embodiments in which the complex tissue is a brain tissue, the organoids produced on a rectangular micropattern comprise cells of both neural and mesodermal lineages.

In some embodiments, a hexagonal pattern involves the growth of six cysts/organoids per micropattern. In some embodiments, cysts/organoids have a diameter of about 100 μm to about 500 μm (e.g., about 150 μm). In some embodiments, a hexagonal pattern has a diameter of about 100 μm to about 500 μm (e.g., about 150 μm). In some embodiments, a hexagonal pattern has a diameter of less than 500 μm or less than 250 μm. In some embodiments, the hexagonal pattern has a minimum intra-hexagon spacing of about 50 μm. In some embodiments, the hexagonal pattern has an intra-hexagon spacing of about 50 μm, 100 μm, 150 μm, 200 μm, 250 μm, 300 μm, 350 μm, 400 μm, 450 μm, 500 μm, 600 μm, 650 μm, 700 μm, 750 μm, or 800 μm. In some embodiments, the hexagonal pattern has an intra-hexagon spacing of at least 50 μm, 100 μm, 200 μm, 300 μm, 400 μm, 500 μm, 600 μm, 700 μm, or 800 μm. In certain embodiments, the hexagonal pattern has an intra-hexagon spacing of less than 400 μm, 300 μm, 200 μm or 100 μm.

In some embodiments, a spatial arrangement of organoids involves elongation along an axis (e.g., along an anterior-posterior axis). In some embodiments, a spatial arrangement of organoids involves (or results in) a posterior tailbud and/or a neural tube. In some embodiments, a spatial arrangement of organoids is designed in order to recapitulate aspects of axial development during culturing and growth of the organoids (e.g., under conditions that promote development) including, but not limited to, axial elongation, single-lumen neural tube formation, traveling segmentation clock waves, and sequential somite segmentation.

In some embodiments, organoids of the disclosure are derived from one or more cell types (including stem cells or progenitor cells). Stem cells (and any other cells) that develop into cysts/organoids (e.g., a stem cell-derived organoid) can be animal stem cells, such as human stem cells. Stem cells can be selected from pluripotent stem cells, multipotent stem cells, oligopotent stem cells, and unipotent stem cells. In some embodiments, stem cells are induced pluripotent stem cells (iPSCs, such as human iPSCs). In some embodiments, stem cells are embryonic stem cells (ESCs). In some embodiments, stem cells are selected from hematopoietic stem cells (HSCs), mesenchymal stem cells (MSCs), neuronal stem cells (NSCs), ectodermal stem cells, endodermal stem cells, or mesodermal stem cells.

In some embodiments, organoids of the disclosure comprise one or more cell types that are common to, or representative of specific complex tissues (e.g., brain or nervous cell types belonging to brain and nervous tissues). In some embodiments, organoids may include one or more cell types selected from axial progenitors, cervical spinal cord progenitors, hindbrain, anterior hindbrain, or neuronal cells. In some embodiments, organoids comprise mesodermal cell clusters and/or neural cell clusters. In some embodiments, axial progenitor cells express at least CDX2. In some embodiments, progenitors of the cervical spinal cord express at least one of HOXB4 and CRABP1. In some embodiments, posterior hindbrain cells express NR2F1. In some embodiments, anterior hindbrain cells express EGR2. In some embodiments, neuronal cells express at least one of HES6 and BTG2. In some embodiments, organoids may include one or more cell types selected from neuromesodermal progenitors, pre-neural tube, neural progenitor, presomatic mesoderm-like cells, early somite cells, mature somite cells, and notochord cells. In some embodiments, the neuromesodermal progenitors express at least one of TBXT and SOX2. In some embodiments, the pre-neural tube cells express at least one of HOXA10, CDX2, and NKX1-2. In some embodiments, the neural progenitors express at least one of SOX1, PAX6, HES5, SOX2, and IRX3. In some embodiments, the presomatic mesoderm-like cell cluster expresses at least one of TBX6, MSGN1, and HES7. In some embodiments, the early somite cell cluster expresses at least one of MEOX1, TCF15, and RIPPLY1. In some embodiments, the mature somite cell cluster expresses at least one of PAX3, TWIST1, and FST. In some embodiments, the notochord cells express at least one of SHH, NOTO, and TBXT.

Coupling of two or more organoids to one another may occur on a substrate. In some embodiments, a substrate is a biocompatible substrate. A biocompatible substrate can be any substrate (e.g., surface, material, or matrix) that is compatible with growth and development of organoids (e.g., stem cell derived organoids). In other words, a biocompatible substrate is a substrate on which organoids can grow and develop under culture conditions that promote development of the organoids. In some embodiments, a biocompatible substrate comprises polystyrene, poly-1-lysine, fibronectin, collagen, gelatin, Matrigel, polyethylene glycol, silicone and/or polydimethylsiloxane (PDMS). A biocompatible substrate can be embedded on a coverslip or in a well (e.g., a sample well in a cell culture plate having 24-wells, 96-wells, etc.).

An organoid for use in any of the methods described herein can be any organoid known to a person of ordinary skill in the art. In some embodiments, an organoid is a brain organoid, an intestinal organoid, a liver organoid, a pancreatic organoid, a kidney organoid, a breast organoid, a lung organoid, or a heart organoid. A brain organoid (also known as a neural organoid) may comprise neurons and glial cells. In some embodiments, a brain organoid is designed to have characteristic similar to the cortex, retina, spinal cord, thalamus, and/or hippocampus.

Organoids are cultured under conditions that promote development of the target patterned tissue following coupling (and/or uncoupling) of two or more organoids to one another based on relative spatial arrangement. Culturing of organoids may involve introduction of conditions that support growth and development of organoids for a period of time. Organoids can be cultured for any period of time necessary to produce an organoid having the target patterned tissue. In some embodiments, culturing of organoids involves culturing under conditions that promote development for at least 1, 2, 3, 4, 5, 6, 7, 8, 9, 10, 14, 21, 28, or 35 days. In some embodiments, culturing of organoids involves culturing under conditions that promote development for 1-50, 5-50, 10-50, 5-45, 5-40, 5-30, 5-25, 5-20, 5-15, 5-10, 10-50, 10-40, 10-30, 10-20, 7-35, 7-28, 7-14 or 5-25 days.

In some embodiments, conditions that promote development of organoids include one or more of basal medium, serum, growth factors, cytokines, and supplements. In some embodiments, organoids are cultured in the presence of basal medium, serum, growth factors, cytokines, and/or supplements. Organoids can be contacted with the basal medium, serum, growth factors, cytokines, and/or supplements. A person of ordinary skill in the art will be able to design a set of culture conditions that promote development and growth based on knowledge of optimal growth conditions for a particular type of organoid (e.g., brain organoid, liver organoid, etc.). In some embodiments, conditions that promote development of organoids may include retinoic acid, bone morphogenetic proteins, ascorbic acid, hormones, antibiotics, antimycotics, buffers, pH adjusters, and attachment factors. Attachment factors are small molecules such as collagen, fibronectin, and laminin that promote the attachment of cells of the organoids (e.g., stem cells belonging to the organoids) to attach to the substrate (e.g., biocompatible substrate). In some embodiments, conditions that promote development of stem cell derived organoids include treatment of the organoids with a differentiation medium. In some embodiments, organoids are contacted with one or more differentiation agents.

In some embodiments, a basal medium is any medium that supports the growth and development of cells (e.g., human stem cells). In some embodiments, a basal medium comprises NB27 media, nutrient broth, or nutrient agar. A basal medium comprises carbohydrates, energy sources, and/or sources of amino acids. In some embodiments, a serum is a supplement comprising hormones, growth factors, and/or attachment factors. In some embodiments, a serum is fetal bovine serum (FBS). Growth factors may include angiopoietin, epidermal growth factor (EGF), ephrin, fibroblast growth factor (FGF), hedgehog proteins, insulin-like growth factor (IGF), neurotrophin, notch factors, platelet-derived growth factor (PDGF), TGF-beta, and/or vascular endothelial growth factor (VEGF). Cytokines may include IFNs (e.g., IFNα, IFNβ, and IFNγ), TNF (e.g., TNF-α and TNF-0), colony stimulating factors (e.g., granulocyte colony-stimulating factor and granulocyte-macrophage colony-stimulating factor), and interleukins (e.g., IL-2).

In some embodiments, a differentiation agent is a WNT agonist (or signaling pathway activator), a TGFβ inhibitor, or a BMP inhibitor. In some embodiments, a WNT agonist comprises CHIR99021, a derivative of CHIR99021, a recombinant WNT3A protein, or a glycogen synthase kinase 3 (GSK3) inhibitor. In some embodiments, a TGFβ inhibitor modulates TGF-β receptor type I kinase (TGF-β RI) signaling. In some embodiments, t a TGFβ inhibitor comprises an ALK5 inhibitor II (e.g., 2-[5-(6-methylpyridin-2-yl)-1H-pyrazol-4-yl]-1,5-naphthyridine), a compound of Formula I as described in U.S. Patent Publication No. 2012/0021519, or a TGF-β receptor inhibitor described in U.S. Patent Publication No. 2010/0267731. In some embodiments, a BMP inhibitor comprises LDN 193189 (4-[6-(4-piperazin-1-ylphenyl)pyrazolo[1,5-a]pyrimidin-3-yl]quinolone), or a compound of Formula I as described in U.S. Patent Publication No. 2011/0053930.

Molecules for use in culturing organoids (e.g., growth factors, cytokines, supplements, retinoic acid, bone morphogenetic proteins, ascorbic acid, hormones, antibiotics, antimycotics, buffers, pH adjusters, attachment factors, and/or differentiation agents) can be used in any reasonable concentration that is known to, or could be derived by a person of ordinary skill in the art. For example, a differentiation agent can be present at a concentration of about between 0.1 μM to 10 μM, or between about 0.2 μM to 9 μM, or about 0.3 μM to 8 μM, or about 0.4 μM to 7 μM, or about 0.5 μM to about 6 μM.

Certain bioengineering methods of the disclosure further comprise culturing micropatterned stem cells on a biocompatible substrate to form cysts (e.g., epithelial cysts), each enclosing a respective single lumen; and exposing the cysts to conditions that confer identity of the organoids. Epithelial cysts, for example, are generally comprised of epithelial cell layers enclosing a lumen (e.g., internal space within the epithelial cysts). In some embodiments, exposing the cysts to conditions that confer identity of the organoids involves culturing the stem cells in the presence of basal medium, serum, growth factors, cytokines, and/or supplements. In some embodiments, stem cells are cultured in the presence of basal medium, serum, growth factors, cytokines, and/or supplements for a period of time necessary to form epithelial cysts. Stem cells can be contacted with the basal medium, serum, growth factors, cytokines, and/or supplements. A person of ordinary skill in the art will be able to design a set of culture conditions that promote development and growth of the stem cells into cysts. In some embodiments, conditions that promote development of stem cells may include retinoic acid, bone morphogenetic proteins, ascorbic acid, hormones, antibiotics, antimycotics, buffers, pH adjusters, attachment factors, and/or differentiation factors.

In some embodiments, bioengineering methods of the disclosure further comprise culturing the cysts (e.g., epithelial cysts) to produce the organoids. In some embodiments, culturing the cysts to produce the organoids involves culturing the cysts in the presence of basal medium, serum, growth factors, cytokines, and/or supplements. In some embodiments, stem cells are cultured in the presence of basal medium, serum, growth factors, cytokines, and/or supplements for a period of time necessary to form cysts. Cysts can be contacted with the basal medium, serum, growth factors, cytokines, and/or supplements. A person of ordinary skill in the art will be able to design a set of culture conditions that promote development and growth of the cysts into organoids. In some embodiments, conditions that promote development of organoids from cysts may include retinoic acid, bone morphogenetic proteins, ascorbic acid, hormones, antibiotics, antimycotics, buffers, pH adjusters, attachment factors, and/or differentiation factors. In some embodiments, the organoids can be further differentiated into the target patterned tissue (e.g., using one or more differentiation agents).

### Methods of Use

Cells of the disclosure (including cells belonging to organoids) can be subjected to methods of nucleic acid sequencing or polypeptide sequencing. Methods of nucleic acid sequencing may comprise methods of RNA sequencing and/or DNA sequencing. In some embodiments, a method of RNA sequencing involves single cell sequencing or ensemble sequencing methods. In certain embodiments, a method of single-cell RNA sequencing involves sequencing the transcriptome of a single cell (e.g., a single cell belonging to an organoid). In some embodiments, a method of RNA sequencing involves determination of RNA sequences present within a single cell (or a population of cells) and/or concentrations of specific RNAs within a single cell (or a population of cells).

Some methods of the present disclosure further comprise perturbing (e.g., disrupting or modulating) gene expression and/or function in cells of the organoids. In some embodiments, gene expression and/or function is perturbed (e.g., disrupted or modulated) prior to development of the target patterned tissue. Changes in the organoids as they develop into the target patterned tissue can be assessed, relative to control organoids (e.g., using methods of nucleic acid sequencing, e.g., methods of single-cell RNA sequencing). Changes in the organoids after they have developed into the target patterned tissue (e.g., following culturing of the organoids to develop the target patterned tissue) can be assessed, relative to control organoids (e.g., using methods of nucleic acid sequencing, e.g., methods of single-cell RNA sequencing).

In some embodiments, perturbing gene expression and/or function comprises delivering gene editing molecules to cells of the organoids. Gene editing molecules can include any gene editing molecules that are known to a person of ordinary skill in the art. In certain embodiments, gene editing molecules can be selected from programmable nucleases and RNA interference molecules. Programmable nuclease may include a CRISPR enzyme (e.g., Cas9), a zinc finger nuclease (ZFN), a transcription activator-like effector nuclease (TALEN), or a meganuclease. A CRISPR enzyme can be a Cas1, Cas2, Cas3, Cas4, Cas5, Cas6, Cas7, Cas8, Cas9, Cas10, Cas11, Cas12, Cas13, or Cas14 enzyme. In some embodiments, a CRISPR enzyme is a dead Cas protein (e.g., dCAS9). In some embodiments, gene editing molecules may involve delivery of a gene of interest to the cells of the organoid (e.g., for expression of the gene of interest in the cells of the organoid. A gene of interest can be delivered to the cells of the organoid using a viral vectors. Such viral vectors can be a vector selected from adenovirus, adeno-associated virus (AAV) vectors, lentivirus vectors, retrovirus vectors, and herpes simplex virus (HSV) vectors. The gene of interest may encode a protein agonist or antagonist. Thus, in some embodiments, gene editing methods are used to deliver a protein agonist or antagonist to cells of the organoids.

Certain bioengineering methods of the disclosure further comprise exposing (e.g., contacting) cells of the organoids to a drug or other chemical agent. In some embodiments, the cells of the organoids are exposed to a drug or other chemical agent prior to development of the target patterned tissue, relative to control organoids. Changes in the development of organoids resulting from exposure of the organoids to the drug or other chemical agent are assessed or determined, in some embodiments. In some embodiments, the cells of the organoids are exposed to a drug or other chemical agent following the development of the target patterned tissue, relative to control organoids. Changes in one or more characteristics of the organoids resulting from exposure of the organoids to the drug or other chemical agent are assessed or determined, in some embodiments.

In some embodiments, the organoids are exposed to a gradient of the drug or other chemical agent. For example, the organoids can be exposed to a gradient, wherein the gradient contains sections with high concentrations of drug or other chemical agent, intermediate concentrations of drug or other chemical agent, and low concentrations of drug or chemical agent. A gradient of concentrations of drug or other chemical agent can be a continuous gradient or a discrete (or stepwise) gradient.

The drug or other chemical agent can be a small molecule (e.g., <900 daltons) or a biomolecule. A small molecule can be any drug-like small molecule or any synthetic organic compound. In some embodiments, a small molecule is a natural product. A biomolecule can be a nucleic acid or protein. In some embodiments, a biomolecule is an antibody, antibody fragment, peptide, lipid, carbohydrate, polymer, RNA interference molecule, antisense molecule, antigen (e.g., peptide antigen). An antibody can be a monoclonal antibody, chimeric antibody, humanized antibody, a single-chain antibody, or a bispecific antibody.

Certain aspects of the disclosure involve high throughput screening methods (e.g., rapid, high throughput screening methods). A high throughput screening method of the disclosure can be performed to determine the effects of exposing stem or progenitor cells or organoids (e.g., organoids in a target pattern) to genetic or chemical perturbations. In some embodiments, the disclosure provides a method of screening (e.g., high throughput screening) comprising coupling organoids to each other based on their relative spatial arrangement (e.g., coupling organoids on a biocompatible multiwell substrate, or in multiple biocompatible multiwell substrates); culturing the organoids under conditions that promote development of the target pattern; exposing the organoids to genetic or chemical perturbation; and assessing changes in development of or in one or more characteristic of the organoids. In other embodiments, the disclosure provides a method of high throughput screening comprising genetically modifying stem cells in suspension (e.g., via viral transduction, e.g., lentiviral transduction); promoting the growth and coupling of organoids to each other based on their relative spatial arrangement (e.g., coupling organoids on a biocompatible multiwell substrate, or in multiple biocompatible multiwell substrates); culturing the organoids under conditions that promote development of the target patterned tissue; and assessing changes in development of or in one or more characteristic of the organoids.

Methods of the disclosure may utilize any of the means of arranging organoids, culturing organoids, exposing organoids to genetic or chemical perturbations, and/or assessing changes in development of the organoids as described herein. In some embodiments, a high throughput method utilizes a multiwell substrate (e.g., cell culture plate having 24-wells, 96-wells). A high-throughput screening method, for example, may involve the use of organoids within each well of a multiwell substrate, wherein each of the organoids is arranged in the same repeating micropattern. In some embodiments, a high-throughput screening method may involve the use of organoids within each well of a multiwell substrate, wherein each of the organoids is arranged in a different repeating micropattern. In some embodiments, each organoid within the high throughput screening method is exposed to a different or unique genetic or chemical perturbation. For example, each organoid can be exposed to a different combination of CRISPR enzyme (e.g. Cas9 enzyme). In some embodiments, each organoid within the high throughput screening method is exposed to the same genetic or chemical perturbation.

### Cell Micropatterning

Micropatterning involves the arrangement of organoids on a micro (or miniature) scale to control the geometry of adhesion of the organoids and substrate rigidity. As described above, in some embodiments, micropatterns can be designed to optimize the development and growth of the organoids into the target patterned tissue. In some embodiments, micropatterns can be designed for the growth of multiple organoids (e.g., for the growth of 2, 3, 4, 5, 6, 7, 8, 9, 10 or more organoids per micropattern). Certain methods of micropatterning involve micropatterning stem cells on a biocompatible substrate, in a predetermined spatial arrangement, wherein the predetermined spatial arrangement promotes development of stem cell-derived organoids into a target patterned tissue.

The relative spatial arrangement of organoids with respect to one another can be determined using any one of the machine learning (ML) models described herein. For example, the relative spatial arrangement of organoids for use in a method of micropatterning can be determined by (a) accessing a trained ML model trained to predict, from multi-scale geometric features determined for a particular organoid in a spatial arrangement of organoids, a characteristic of the particular organoid; and (b) identifying the at least one spatial arrangement of organoids that promotes development of the organoids into the target patterned tissue by using a stochastic optimization technique in which spatial arrangements of organoids are evaluated using the trained ML model.

Certain embodiments of a method of micropatterning further comprise using photolithography to fabricate the substrate with wells to hold signal-secreting beads at desired locations; using microprinting to stamp shapes (e.g., circles or polygonal shapes) of extracellular matrix protein on the substrate; depositing stem cells onto the substrate; and exposing the stem cells to media containing extracellular matrix protein (e.g., Matrigel™) to produce cysts, each enclosing a respective single lumen. For example, signal-secreting beads can be deposited into wells of a multiwell substrate in such a manner as to generate signaling gradients (e.g., continuous or stepwise gradients) on the substrate.

Shapes of extracellular matrix protein on a substrate may comprise about 50 to 150 shapes, about 50 to 200 shapes, about 25 to 100 shapes, about 50 to 300 shapes, or about 50 to 500 shapes. In some embodiments, shapes of extracellular matrix proteins have a diameter (or width or length) of about 50 to 200 microns. In some embodiments, shapes of extracellular matrix proteins have a diameter (or width or length) of about 50-500 μm, 50-400 μm, 50-300 μm, 50-200 μm, 50-100 μm, 100-500 μm, or 100-200 μm.

A biocompatible substrate for use in a method of micropatterning can be any substrate (e.g., surface, material, or matrix) that is compatible with growth and development of organoids (e.g., stem cell derived organoids). In some embodiments, a biocompatible substrate comprises polystyrene, poly-1-lysine, fibronectin, collagen, gelatin, Matrigel™, polyethylene glycol, silicone and/or polydimethylsiloxane (PDMS). A biocompatible substrate can be embedded on a coverslip or in a well (e.g., a sample well in a cell culture plate having 24-wells, 96-wells, etc.).

Microprinting (also referred to as microcontact printing) can be used for transferring a monolayer of protein onto a substrate, such as a glass surface or another biocompatible surface. In some embodiments, the microcontact printing occurs using lithography, such as photolithography or soft lithography. In some embodiments, microcontact printing is used to produce a stamp and/or chip/device for the production of organoids. The patterns or designs utilized on the stamp and/or chip can be modified depending on the culture conditions and the intended use of the organoids.

In some embodiments, a stamp is produced having a predetermined spatial arrangement, such as for a target organoid pattern. In some embodiments, the pattern on the stamp is in a grid format. In other embodiments, the pattern on the stamp can be any pattern described herein. In some aspects, the stamp pattern comprises multiple arrays and within each array is a number of shapes. One example of a stamp pattern can be an 8 by 8 array consisting of sets of 4 shapes. In another example of a stamp pattern the stamp may comprise a 6 by 4 array consisting of 36 shapes. In some embodiments, the stamp is a polydimethylsiloxane (PDMS) stamp. In some aspects, a PDMS stamp is contacted or coated with extracellular matrix protein (e.g., Matrigel™) or other protein mixtures.

In some embodiments, the stamp, e.g., the PDMS stamp, is applied to a surface to transfer material, such as a protein mixture, from the stamp to the surface. In some aspects, the surface is a glass coverslip. In other embodiments, the surface is a substrate, such as a biocompatible substrate. In some aspects, the substrate comprises a thermoplastic material. In some embodiments, the substrate comprises PDMS, e.g., a PDMS chip. In one embodiment, a PDMS stamp is applied to a PDMS chip to transfer one or more proteins between the two surfaces. In some embodiments, the application of a PDMS stamp to a PDMS chip utilizes humified microcontact printing or a hydrophilized microcontact printing method.

In some aspects, humified microcontact printing comprises obtaining a PDMS stamp having a pre-determined pattern and obtaining a PDMS flat stamp. The PDMS patterned stamp can be plasma treated and extracellular matrix protein (e.g., Matrigel™) can be applied to the PDMS flat stamp. The plasma treated PDMS patterned stamp is then pressed onto the flat PDMS stamp to allow the positive features to be coated with extracellular matrix protein (e.g., Matrigel™). The extracellular matrix protein (e.g., Matrigel™) coated PDMS stamp can be applied to the PDMS chip and water is injected between the PDMS stamp and PDMS chip. In some aspects, the water outcompetes the adhesion of matrix proteins on the PDMS stamp (e.g., the activated high surface energy stamp surface) and these proteins are displaced onto the PDMS chip or device (e.g., the low surface energy surface). In some aspects, the PDMS stamps and/or chips can be designed to further include water channels, e.g., to enhance the diffusion of water molecules between the stamp and the chip or device.

### Computer Implementation

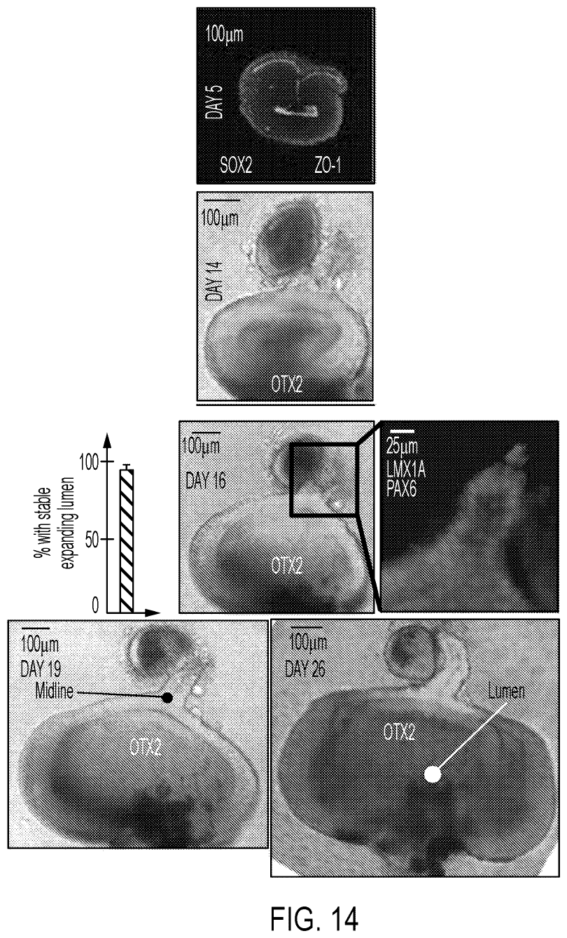

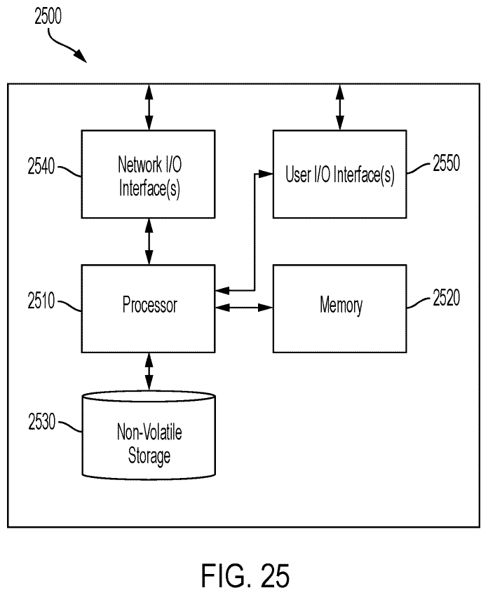

An illustrative implementation of a computer system 2500 that may be used in connection with any of the embodiments of the technology described herein (e.g., such as the method of FIG. 17) is shown in FIG. 25. The computer system 2500 includes one or more processors 2510 and one or more articles of manufacture that comprise non-transitory computer-readable storage media (e.g., memory 2520 and one or more non-volatile storage media 2530). The processor 2510 may control writing data to and reading data from the memory 2520 and the non-volatile storage device 2530 in any suitable manner, as the aspects of the technology described herein are not limited to any particular techniques for writing or reading data. To perform any of the functionality described herein, the processor 2510 may execute one or more processor-executable instructions stored in one or more non-transitory computer-readable storage media (e.g., the memory 2520), which may serve as non-transitory computer-readable storage media storing processor-executable instructions for execution by the processor 2510.

Computing device 2500 may also include a network input/output (I/O) interface 2540 via which the computing device may communicate with other computing devices (e.g., over a network), and may also include one or more user I/O interfaces 2550, via which the computing device may provide output to and receive input from a user. The user I/O interfaces 2550 may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

### Additional Embodiments

The present disclosure further provides embodiments encompassed by the following numbered paragraphs:
1. A method of using a trained machine learning (ML) model to identify a spatial arrangement of stem cell aggregates, the spatial arrangement for promoting development of the stem cell aggregates into target patterned tissue, the method comprising:
   using at least one processor to perform:
      accessing the trained ML model, the trained ML model being trained to predict, using a feature vector generated for an input spatial arrangement of the stem cell aggregates, a patterning metric associated with the input spatial arrangement; and
      identifying a spatial arrangement of stem cell aggregates that optimizes the patterning metric by using a stochastic optimization technique in which different spatial arrangements of stem cell aggregates are evaluated using their respective patterning metrics as predicted by applying the trained ML model to feature vectors generated for the different spatial arrangements.
2. The method of paragraph 1, wherein the different spatial arrangements include a first spatial arrangement, wherein applying the trained ML model to feature vectors generated for the different spatial arrangements comprises:
   generating a first feature vector for the first spatial arrangement; and
   providing the first feature vector as input to the trained ML model to obtain corresponding output including a first patterning metric for the first spatial arrangement.
3. The method of paragraph 2, wherein generating the first feature vector for the first spatial arrangement of stem cell aggregates comprises generating a vector including one or more values indicative of:
   one or more densities for one or more stem cell aggregates in the first spatial arrangement,
   one or more density gradients for one or more stem cell aggregates in the first spatial arrangement,
   one or more higher-order derivative of densities for one or more stem cell aggregates in the first spatial arrangement,
   an image of the first spatial arrangement,
   one or more Fourier transform components of the image of the first spatial arrangement,
   when the first spatial arrangement includes at least one bead,
      position of one or more beads within the first spatial arrangement, and/or
      type of the one or more beads within the first spatial arrangement, and/or one or more shapes of one or more stem cell aggregates in the first spatial arrangement.
4. The method of paragraph 2, wherein generating the first feature vector comprises generating a vector including:
   one or more density values computed, at one or more respective length scales and using information specifying the first spatial arrangement of stem cell aggregates, for one or more stem cell aggregates in the first spatial arrangement of stem cell aggregates; and/or
   one or more density gradient values computed, at one or more respective length scales and using information specifying the first spatial arrangement of stem cell aggregates, for one or more stem cell aggregates in the first spatial arrangement of stem cell aggregates.
5. The method of paragraph 4, wherein generating the first feature vector comprises generating a vector having a density gradient value computed at a length scale between 50 and 300 microns, optionally 200 microns, and/or a density value computed at a length scale between 500 and 1000 microns, optionally 700 microns.
6. The method of paragraph 2, wherein generating the first patterning metric using the trained ML model comprises generating one or more values indicative of a predicted expression for each of one or more biomarkers associated with patterned tissue to be generated using stem cell aggregates having the first spatial arrangement.
7. The method of paragraph 2, wherein generating the first patterning metric comprises generating one or more values indicative of:
   a polarization of predicted expression of a biomarker,
   a dipole moment of the predicted expression of the biomarker,
   a multipole moment of the predicted expression of the biomarker,
   a spatiotemporal pattern of the expression, polarization, dipole moment, and/or multipole moment of the predicted expression of the biomarker,
   predicted expression and/or predicted pattern of expression of cell signals (e.g., secreted biomarker(s)), and/or
   morphology of the first stem cell aggregate and/or tissue to be generated using stem cell aggregates having the first spatial arrangement.
8. The method of any one of paragraphs 1-7, wherein the trained ML model is trained to predict, using the feature vector generated for a particular stem cell aggregate in the input spatial arrangement of stem cell aggregates, a respective patterning metric for the particular stem cell aggregate.
9. The method of any one of paragraphs 1-7, wherein the trained ML model is trained to predict, using the feature vector generated for the input spatial arrangement of stem cell aggregates, a respective patterning metric for each of multiple stem cell aggregates in the input spatial arrangement of stem cell aggregates.
10. The method of any one of paragraphs 1-9, wherein the trained ML model comprises a regression model.
11. The method of paragraph 10, wherein the regression model comprises a linear regression model.
12. The method of paragraph 10, wherein the regression model comprises a non-linear regression model.
13. The method of paragraph 12, wherein the non-linear regression model comprises a kernel regression model, optionally wherein the kernel regression model uses a linear or radial basis function (RBF) kernel.
14. The method of paragraph 10, wherein the regression model comprises a support vector machine (SVM) regression model, optionally wherein the SVM regression model uses a linear or RBF kernel.
15. The method of paragraph 10, wherein the regression model comprises a neural network model.
16. The method of paragraph 15, wherein the neural network model has a single layer or multilayer architecture, optionally a convolutional neural network architecture.
17. The method of any one of paragraphs 1-16, further comprising:
   obtaining training data comprising, for each particular spatial arrangement of multiple spatial arrangements,
      (i) at least one measured value indicative of a patterning metric for the particular spatial arrangement,
      (ii) a feature vector generated from information specifying the particular spatial arrangement; and
   estimating values of parameters of an ML model using the obtained training data to obtain the trained ML model.
18. The method of paragraph 17, further comprising:
   generating the training data at least in part by:
      generating multiple random patterns;
      culturing stem cell aggregates on one or multiple two-dimensional substrates and/or or in a three-dimensional matrix in accordance with the generated respective multiple random patterns;
      obtaining images of the cultured stem cell aggregates; and
      generating the training data from the obtained images by:
         generating, from one or more of the obtained images, patterning metric values for spatial arrangements in the one or more of the obtained images; and
         generating, from the one or more of the obtained images, feature vectors for spatial arrangements in the one or more of the obtained images.
19. The method of paragraph 18, wherein obtaining the images of the cultured stem cell aggregates comprises obtaining one or more antibody-stained, in situ probe-hybridized RNA, and/or live fluorescent reporter images of the cultured stem cell aggregates corresponding to each of one or more biomarkers.
20. The method of any one of paragraphs 1-19, wherein using the stochastic optimization technique comprises:
   obtaining a first spatial arrangement of stem cell aggregates and determining a first patterning metric for the first spatial arrangement using the trained ML model;
   modifying the first spatial arrangement to generate a second spatial arrangement of stem cell aggregates different from the first spatial arrangement;
   determining a second patterning metric for the second spatial arrangement using the trained ML model; and
   evaluating the second patterning metric relative to the first patterning metric.
21. The method of paragraph 20, wherein using the stochastic optimization technique comprises,
   obtaining a plurality of patterning metrics by iteratively:
      generating a spatial arrangement from a previously generated spatial arrangement, and
      determining a patterning metric for the spatial arrangement; and
   identifying, from among the plurality of patterning metrics, one or more optimized patterning metrics.
22. The method of paragraph 20 or 21, wherein using the stochastic optimization technique comprises using simulated annealing with acceptance defined as a function of output of the trained ML model.
23. The method of paragraph 19 or 20, wherein using the stochastic optimization technique comprises using an iterative descent algorithm, optionally a gradient descent algorithm, with a step direction being a function of output of the trained ML model.
24. A system, comprising:
   at least one computer hardware processor; and
   at least one non-transitory computer readable storage medium storing processor executable instructions that, when executed by the at least one computer hardware processor, cause the at least one computer hardware processor to perform the method of any one of paragraphs 1-17 and 19-23.
25. At least one non-transitory computer readable storage medium storing processor executable instructions that, when executed by at least one computer hardware processor, cause the at least one computer hardware processor to perform the method of any one of paragraphs 1-17 and 19-23.
26. A bioengineering method for producing a target patterned tissue (e.g., human tissue), the method comprising:
   coupling organoids to each other based on their relative spatial arrangement, optionally in one, two, or three dimensions; and
   culturing the organoids under conditions that promote development of the target patterned tissue.
27. The bioengineering method of paragraph 26, wherein the spatial arrangement is determined using the method of any one of paragraphs 1-25.
28. The bioengineering method of 26 or 27, wherein the organoids are derived from stem cells micropatterned on a biocompatible substrate in a predetermined spatial arrangement, and wherein the predetermined spatial arrangement promotes development of organoids into the target patterned tissue.
29. The bioengineering method of paragraph 28, wherein the stem cells are animal stem cells.
30. The bioengineering method of paragraph 29, wherein the animal stem cells comprise human stem cells or chimeric human/non-human cells.
31. The bioengineering method of paragraph 28 or 30, wherein the stem cells are selected from pluripotent stem cells, multipotent stem cells, oligopotent stem cells, and unipotent stem cells.
32. The bioengineering method of any one of paragraphs 28-31, wherein the stem cells are induced pluripotent stem cells (iPSCs).
33. The bioengineering method of any one of paragraphs 28-31, wherein the stem cells are embryonic stem cells (ESCs).
34. The bioengineering method of any one of paragraphs 28-31, wherein the stem cells are selected from hematopoietic stem cells (HSCs), mesenchymal stem cells (MSCs), intestinal stem cells (ISCs), neuronal stem cells (NSCs), ectodermal stem cells, mesodermal stem cells, endodermal stem cells, or extraembryonic stem cells.
35. The bioengineering method of any one of paragraphs 28-34, wherein the biocompatible substrate comprises polystyrene, borosilicate glass, poly-1-lysine, fibronectin, collagen, laminin, gelatin, Matrigel, agarose, alginate, polyethylene glycol, silicone and/or polydimethylsiloxane (PDMS).
36. The bioengineering method of any one of paragraphs 28-33, wherein the predetermined spatial arrangement is a geometric pattern.
37. The bioengineering method of paragraph 36, wherein the geometric pattern is a triangular, quadrilateral, pentagonal, hexagonal, heptagonal, octagonal, nonagonal, or decagonal.
38. The bioengineering method of paragraph 36 or 37, wherein the geometric pattern includes a region for a central organoid.
39. The bioengineering method of any one of the paragraphs 28-38, wherein the stem cells from which each organoid is derived are deposited on the substrate in a polygonal shape, optionally a mathematically parametrized polygonal shape, such as a trapezoid parametrized by the lengths of the parallel edges and spacing between these edges.
40. The bioengineering method of any one of the paragraphs 26-39, wherein the spatial arrangement of organoids includes an arrangement of organoids having different shapes (e.g., a mixture of different shapes of organoids).
41. The bioengineering method of any one of paragraphs 26-40, wherein the distance between the center of any two or more of the organoids on a biocompatible substrate is about 100 microns to about 1000 microns.
42. The bioengineering method of any one of paragraphs 26-41, wherein the diameter, minimum length, or minimum width of each of the organoids is about 100 microns to about 1000 microns, or wherein the area occupied by each of the organoids is about 5,000 μm² to about 500,000 μm².
43. The bioengineering method of any one of paragraphs 26-42, wherein the organoid is a brain organoid, an intestinal organoid, a liver organoid, a pancreatic organoid, a kidney organoid, a breast organoid, a lung organoid, or a heart organoid, or a gastruloid with multiple germ layer tissues that are axially and/or mediolaterally and/or dorsoventrally patterned, or an organoid derived from ectodermal, mesodermal, endodermal, and/or extraembryonic progenitors.
44. The bioengineering method of any one of paragraphs 16-27, wherein the organoid is derived from ectodermal, mesodermal or endodermal progenitors.
45. The bioengineering method of any one of paragraphs 26-45, wherein the conditions that promote development of the target patterned tissue include culture and/or differentiation conditions, wherein the differentiation conditions include one or more of basal medium, serum, growth factors, cytokines, supplements, optionally selected from retinoic acid (RA), morphogens including bone morphogenetic protein (BMP), fibroblast growth factor (FGF), transforming growth factor beta (TGFβ), NODAL, WNT, sonic hedgehog (SHH), or equivalent small molecule agonists and/or antagonists of these pathways, and/or vitamins, hormones, antibiotics, antimycotics, buffers, pH adjusters, pH indicators, and attachment factors, optionally selected from collagen, fibronectin, laminin, and Matrigel™.
46. The bioengineering method of any one of paragraphs 28-45-, further comprising:
   culturing the micropatterned stem cells on the biocompatible substrate to form organoids; and
   exposing the organoids to extracellular matrix protein and differentiation conditions.
47. The bioengineering method of paragraph 30 further comprising differentiating the organoids into the target patterned tissue.
48. The bioengineering method of paragraph 47, further comprising removing the organoids from the patterned substrate and culturing them under low-adherent conditions (e.g., on a low-adherent single-well or multi-well plate), optionally for multiple days, weeks, or months.
49. The bioengineering method of any one of paragraphs 26-48, further comprising performing single-cell RNA sequencing, spatial transcriptomic profiling, in situ hybridization or immunostaining on cells from the organoids.
50. The bioengineering method of any one of paragraphs 26-49, further comprising perturbing gene expression and/or function in cells of the organoids, optionally prior to development of the target patterned tissue, and assessing changes in development of the organoids into the target patterned tissue, relative to control (e.g., unperturbed) organoids.
51. The bioengineering method of paragraph 50, wherein the perturbing gene expression and/or function comprises delivering gene editing molecules to cells of the organoids.
52. The bioengineering method of paragraph 50 or 51, comprises a transfer of the cells of the organoids (optionally wherein the cells are in suspension) to wells of a multiwell (e.g., 96 well) plate and perturbing gene expression and/or function in the cells.
53. The bioengineering method of paragraph 51 or 52, wherein the gene editing molecules are selected from (i) programmable nucleases, optionally, a CRISPR based enzyme (e.g., CRISPR, CRISPRi, CRISPRa), a zinc finger nuclease (ZFN), a transcription activator-like effector nuclease (TALEN), or a meganuclease, and (ii) RNA interference molecules.
54. The bioengineering method of paragraph 53, wherein the gene editing molecules are selected from viral vectors, optionally adenovirus, adeno-associated virus (AAV) vectors, lentivirus vectors, retrovirus vectors, and herpes simplex virus (HSV) vectors, wherein the viral vectors comprise and/or express a molecule that perturbs genetic expression (e.g., gRNA or RNAi).
55. The bioengineering method of any one of paragraphs 50-54, wherein the perturbing gene expression and/or function comprises delivering a protein agonist or antagonist to cells of the organoids.
56. The bioengineering method of any one of paragraphs 26-55, further comprising exposing cells of the organoids to a drug or other chemical agent, optionally gradients of the drug or other chemical agent.
57. The bioengineering method of any one of paragraphs 26-55, further comprising exposing cells of the organoids (optionally wherein the cells are in suspension) to a drug or other chemical agent, optionally gradients of the drug or other chemical agent, prior to development of the target patterned tissue, and assessing changes in development of the organoids into the target patterned tissue, relative to control organoids.
58. The bioengineering method of paragraph 57, further comprises transfer of the organoids to a multiwell (e.g., 96 well) plate and exposing cells of the organoids to a drug or other chemical agent.
59. The bioengineering method of any one of paragraphs 26-58, further comprising exposing cells of the organoids to a drug or other chemical agent, optionally gradients of the drug or other chemical agent, after development of the target patterned tissue, and assessing changes in one or more characteristic of the organoids of the target patterned tissue, relative to control organoids.
60. The bioengineering method of any one of paragraphs 56-59, wherein the drug or other chemical agent is selected from small molecule drugs (e.g., <900 daltons) and antibodies (including antibody fragments).
61. A cell micropatterning method, comprising:
   micropatterning stem cells on a biocompatible substrate, in a predetermined spatial arrangement, wherein the predetermined spatial arrangement promotes development of organoids into a target patterned tissue, wherein the spatial arrangement is determined using the method of any one of paragraphs 1-25.
62. The method of paragraph 61, wherein the substrate comprises polystyrene, poly-1-lysine, fibronectin, collagen, gelatin, Matrigel, polyethylene glycol, silicone and/or polydimethylsiloxane (PDMS).
63. The method of paragraph 62, wherein the substrate is patterned with capture molecules (e.g., DNA, RNA or protein) capable of binding to complementary or cognate target molecules in the stem cells.
64. The method of paragraph 61 or 62, comprising:
   using photolithography to fabricate the substrate with microwells to hold signal-secreting beads at desired locations;
   using a stamp (optionally a three-layer stamp) comprising water channels and a repeating micropattern to microprint shapes (e.g., circles or polygonal shapes, such as trapezoids; e.g., about 50 to 150 shapes) of extracellular matrix protein on the substrate, wherein the repeating micropattern on the stamp is aligned between the microwells;
   depositing stem cells onto the substrate; and
   exposing the stem cells to media containing extracellular matrix protein to produce cysts, each enclosing a respective lumen.
65. The method of paragraph 64 further comprising selectively depositing one or more signal-secreting or inhibitor-secreting beads into the microwells, thereby exposing the cysts to signaling gradients on the substrate, and optionally removing and/or redepositing the beads at different times.
66. The method of paragraph 65, wherein the beads are selectively injected into the microwells using a manual injector featuring a glass capillary controlled by pneumatic pressure.
67. The method of paragraph 64 or 65, wherein placement of the one or more beads in the microwells relative to the cysts is determined using the method of any one of paragraphs 1-25.
68. A method of producing one or more organoids comprising:
   seeding a population of pluripotent stem cells on a surface in a repeating pattern; and differentiating the deposited pluripotent stem cells to form one or more organoids.
69. A method of producing one or more organoids comprising:
   seeding a population of pluripotent stem cells on a micropatterned substrate in a repeating pattern; and
   differentiating the deposited pluripotent stem cells to form one or more organoids.
70. A high throughput method of perturbing organoids comprising:
   preparing lentivirus in a multiwell (optionally a 6, 12, 24, 96, or 384 well) plate, wherein each well of the multiwell plate comprises an agent for genetic perturbation (e.g., knock down, knockout, or overexpression);
   printing one or more patterns on a surface;
   placing a removable well system on the surface, thereby separating the one or more patterns from one another;
   combining the lentivirus with stem cells to form a mixed cell suspension;
   depositing the mixed cell suspension on the one or more patterns;
   differentiating deposited cells of the suspension to form one or more organoids; and
   assessing changes in development of, or in one or more characteristic of, the organoids.
71. A rapid, high throughput screening method, comprising:
   on a biocompatible multiwell substrate or on multiple biocompatible multiwell substrates, coupling organoids to each other based on their relative spatial arrangement, optionally wherein the substrate is fabricated using the method of any one of paragraphs 61-67;
   culturing the organoids under conditions that promote development of a target patterned tissue;
   exposing the organoids to cell signals, or to genetic or chemical perturbation; and
   assessing changes in development of, or in one or more characteristic of, the organoids.
72. The screening method of paragraph 71, wherein each of the biocompatible multiwell substrates comprises a repeating micropattern of extracellular matrix protein and organoids.
73. The method of paragraph 71 or 72, wherein exposing the organoids to cell signals or chemical perturbation comprises depositing signal-secreting beads or chemical-coated beads, respectively, into the microwells.
74. The method of paragraph 71 or 72, wherein exposing the organoids to genetic perturbation comprises delivering gene editing agents to the organoids.

### EXAMPLES

### Example 1. Identifying Patterns of Organoids that Facilitate Developmental Processes

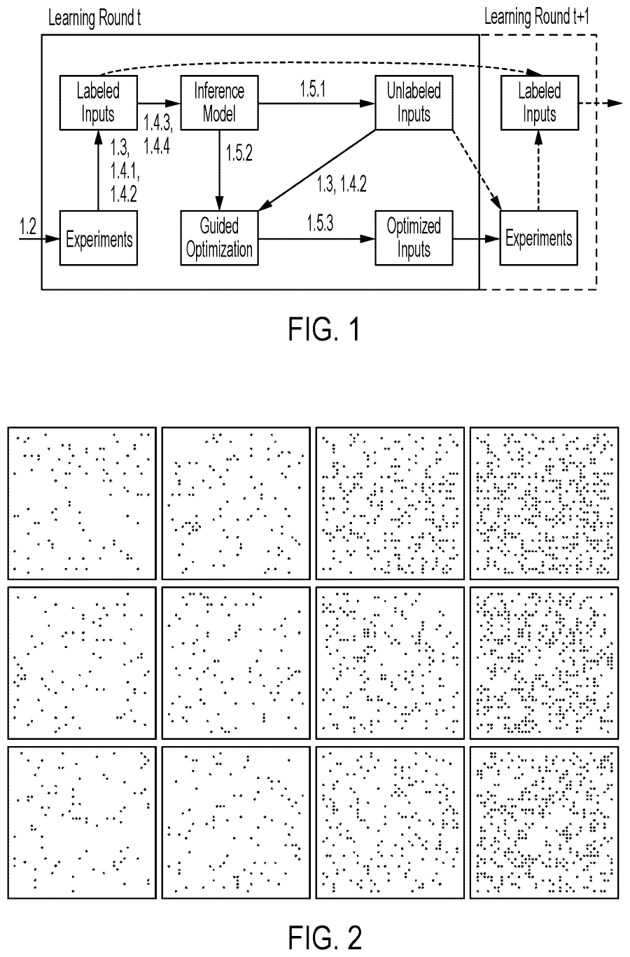

One of the objectives of the end-to-end machine learning pipeline provided herein is to reproducibly and scalably induce a development process of interest, such as axial elongation exemplified below (Example 2), in organoids and other cell aggregate structures by controlling their spatial coupling. In this Example, which can be applied to other developmental processes of interest, we show how to generate and process data to spatially couple organoids. We also expand upon our model and framework used for identifying patterns of organoids that facilitate developmental processes of interest (e.g., lead to symmetry breaking in the context of axial elongation). A diagram of at least some of the components of this pipeline is shown in FIG. 1. In our work, the model was able to optimize spatial coordinates within one round of learning. However, this pipeline can be generalized for other use cases that span multiple rounds of learning.

As discussed in greater detail in Example 2 below, an initial step of the framework provided herein is to obtain distinct random arrangements of cellular cysts on each substrate, each with a different average density (number of cysts per unit area). To generate organoids, we expose these cysts to conditions that confer a cell or tissue characteristic. To generate the organoids in Example 2, for example, we exposed the cysts to conditions that confer a posterior epiblast identity by activating the canonical WNT pathway using the GSK3B inhibitor CHIR99021 (CHIR) while inhibiting the BMP and TGFβ pathways using the SMAD inhibitors LDN193189 (LDN) and A8301 respectively. Following several days of differentiation, we stain the differentiated organoids for one or more particular biomarker(s) indicative of the characteristic of interest. Again, using Example 2 as an example, we stained the differentiated organoids for CDX2, a posterior marker expressed by axial progenitors in the tailbud and SOX1, an anterior marker expressed by cells in the neural tube. Other pairs of markers could be used. Under fixed differentiation conditions, the proportion of anterior SOX1+ versus posterior CDX2+ fates on a substrate was positively correlated with average organoid density.

Data generated from experiments (section 1.2) is featurized and labeled (sections 1.3, 1.4.1, 1.4.2). Processed data is then inputted into an inference model (1.4.3, 1.4.4) for training. Subsequently, the model is involved in sampling unlabeled inputs for optimization (1.5.1). The model then guides optimization (1.5.2) of sampled inputs. Finally, the optimized inputs are post-processed for the next round of experiments (1.5.3). Details specific to our learning objective are elaborated in the sections below. If necessary, this process can be iterated upon in a subsequent round as represented by the dashed lines in the diagram.

### 1.2 Data Generation

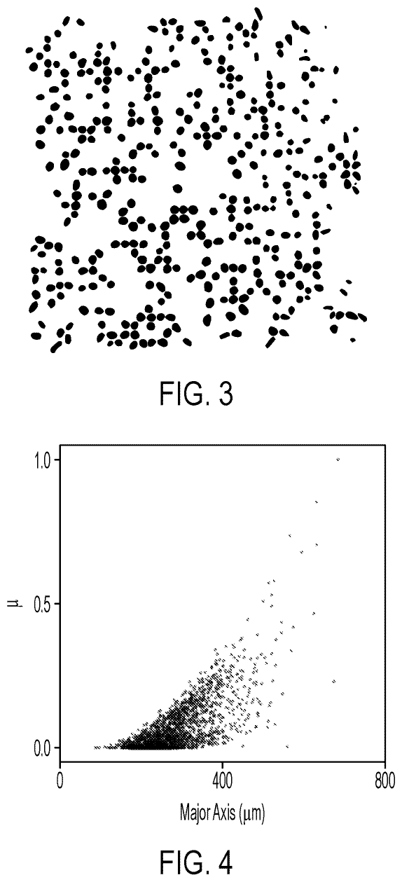

We computationally designed 2D random micropattern arrays with markers indicating the spatial position of organoids. The diameter of each marker was set to 150 pixels to reflect the diameter of organoids in microns in all experiments. This diameter was kept fixed in all experiments—smaller diameters were more difficult to print reliably, and larger diameters were avoided due to the emergence of organoids with multiple lumens. The minimum spacing between neighboring markers was set to 50 pixels to prevent merging of neighboring organoids. All micropattern arrays were created using the numpy library in Python. To sample a wide variety of pattern densities, for each 2D array, we randomly placed a marker at position i, j in an 8000 by 8000-pixel grid for each of 12 probabilities evenly sampled between 1/16 and 1/4, inclusively (FIG. 2). The 12 generated random arrays contained a total of 2456 markers. These random array designs were then used to define organoid coordinates in all experiments that were carried out.

### 1.3 Data Processing

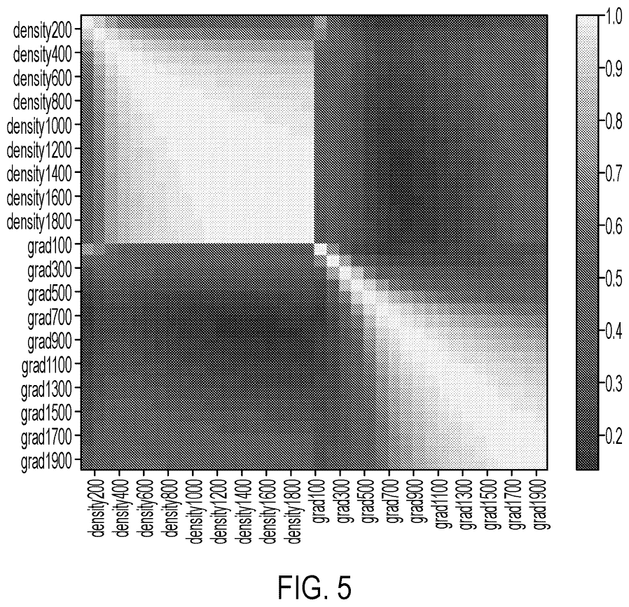

After performing experiments, to build a dataset for the regression model, we processed DAPI, CDX2 and SOX1 stained images associated with the 12 previously generated micropattern arrays. DAPI-stained images were segmented using FIJI software by converting distances in pixels to microns, applying a Gaussian blur of 10 microns, thresholding the image using the Renyi entropy method followed by the watershed algorithm for segmentation (see, e.g., FIG. 3). In cases where automated binary watershed tool failed, the pencil tool set to a thickness of 1-2 pixels was used to manually separate objects, using the DAPI channel as a guide. Objects were manually split or merged to correct errors. Merged objects were not included in further analysis and were removed from the segmented image. This process was followed for segmenting all immunostain images in this manuscript.

Due to experimental errors, some DAPI images contained excess or missing organoids. For such images, we manually annotated segmented images in FIJI. To ensure that the number of markers in the micropattern arrays agreed with the number of organoids in the DAPI images, we (i) removed any organoids from the segmented DAPI images where markers were not present in micropattern arrays and (ii) removed any markers from the micropattern arrays where organoids were missing in the segmented DAPI images. At the end of this process, a total of 2373 segmented organoids were obtained.

Finally, the coordinates of markers in the micropattern arrays were matched with the coordinates of organoids in segmented DAPI images by rotationally aligning the images in FIJI, computing centroids of organoids using the Open-CV library, and algorithmically matching centroid coordinates between micropattern arrays and segmented images. Remaining errors were corrected manually.

### 1.4 Feature Design and Selection

We computed a polarization p for each organoid to measure the degree of A-P symmetry breaking. We also modeled diffusible signals produced by organoids by fixing Gaussians on marker positions at various length scales denoted by σ. We computed features density p and gradient ∥∇ρ∥ at each marker position to construct a training data set. Finally, we used a kernel regression model to predict polarization. The model's features were chosen according to a sequential forward selection algorithm.

### 1.4.1 Polarization μ

The polarization μ for a given organoid was defined as the dipole moment of CDX2 intensity,

$$μ = {\sum }_{i = 1}^{N} ( {c}_{i} - \overline{c} ) {g}^{i}$$

where N is the number of pixels within the segmented DAPI contour of an organoid, cᵢ={x, y} is the ith pixel coordinate within the contour, c̅={x̅, y̅} is the centroid of an organoid in the segmented DAPI image, and gⁱ is the binarized pixel intensity of the associated CDX2 immunostain image at the ith coordinate. The immunostain image was binarized using FIJI at a pixel intensity threshold that separated the bimodal CDX2 intensity distribution.

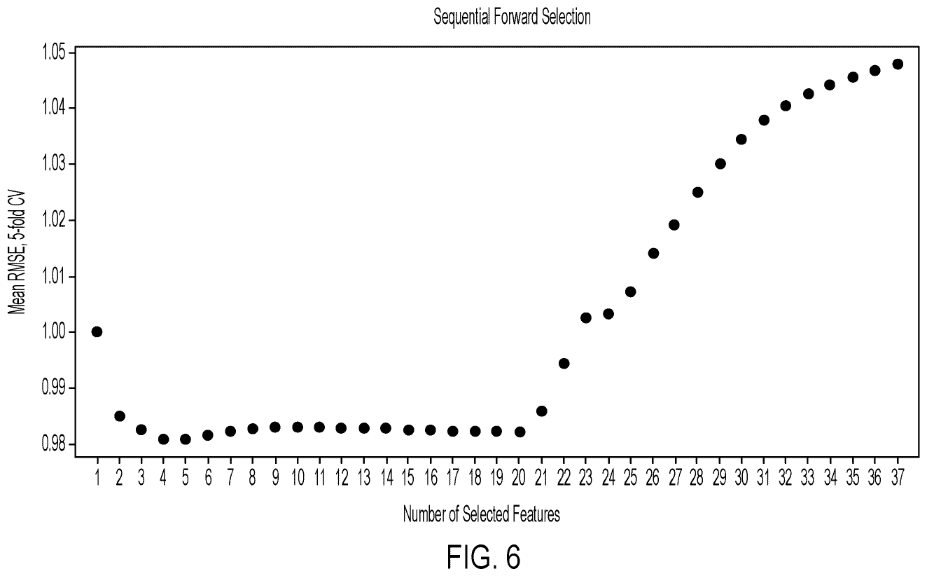

The measured polarization was highly correlated with major axis length across organoids (r=0.75, n=2373) (FIG. 4). This was intentional, so that the degree of A-P axis formation and elongation could be represented in a single metric, simplifying learning.

### 1.4.2 Density ρ and Gradient ∥∇ρ∥

To enumerate the possible length scales of diffusion, Gaussian smoothing was applied across all patterns with σ∈{100, 200, . . . , 2000}. Specifically, the 100-to-2000-micron range was chosen as follows. 100 microns was chosen as a lower bound, as organoids themselves were 150 microns in diameter. An interval of 100 microns was chosen due to the high correlation of features with consecutive values of sigma. 2000 microns was chosen as an upper bound, as above this value, features were so highly correlated with all other features that they captured no information.

For each marker index j in each blurred micropattern array, the density

$${ρ}_{σ}^{j}$$

with respect to a was defined as,

$${ρ}_{σ}^{j} = \frac{1}{n} {\sum }_{i = 1}^{n} {s}_{i}^{j}$$

where n is the total number of pixels in each marker and sᵢ is the pixel intensity of the ith pixel in the marker. Note that n is fixed across all markers since the area of each marker is the same. The gradient ∥∇ρ_{σ}∥^{j} was defined as,

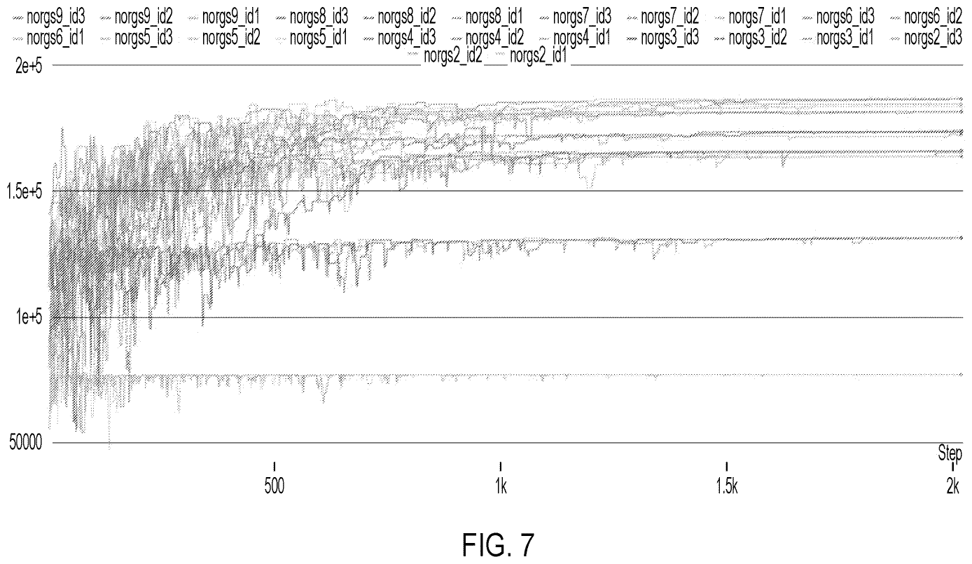

∥∇ρ_{σ}∥^{j}={square root over (((sᵣ−sᵢ)/r)²+((sᵤ−s_{d})/r)²)}

where sᵣ, sᵢ, sᵤ, and s_{d} are the intensities of the rightmost, leftmost, uppermost and downmost pixel in the marker with respect to the centroid c̅ and r=75 its radius, respectively. Density ρ_{σ} and gradient ∥∇ρ_{σ}∥^{j} features were computed for all markers in all random micropattern arrays across all values of σ, resulting in a data set with 40 candidate features in total across 2373 organoids. See FIG. 5.

### 1.4.3 Feature Selection

The sequential forward selection algorithm was used to select features from the candidate pool of features described in 1.4.2. Sequential forward selection takes as input a model for prediction and a pool of features for selection. At each step, the prediction model chooses the best feature to add according to cross-validation (CV) score in greedy fashion. The algorithm is stopped when k features are selected. Sequential forward selection was performed using 5-fold CV with the root mean square error scoring function. Sequential forward selection was repeated for each k, varying from 1 to the total number of candidate features (40). Based on these results, k*=2 features, ρ700 (density at a length scale of 700 microns) and ∥∇ρ200∥ (gradient at a length scale of 200 microns), were chosen according to the elbow method. Additionally, the choice of 2 features was sufficient, as there was a high degree of correlation of subsequently selected features with the features already chosen. For example, the third selected feature was ∥ρ100∥ (gradient at a length scale of 100 microns), which had a correlation of r=0.7 with ∥∇ρ200∥.

### 1.4.4 Prediction Model

The polarization predictions \hat{μ} were made by a kernel ridge regression model. Kernel ridge regression combines ordinary least squares regression with an L2 regularization and a kernel matrix K∈ [symbol] ^{n×n}. K is defined as K_{i,j}=K(xᵢ, xⱼ) where K is a kernel function that measures similarity between input feature vectors xᵢ and xⱼ. The optimization task can be posed as follows,

$${min}_{θ ∈ {ℝ}^{d}} \{ {\sum }_{i = 1}^{N} {( {μ}_{i} - f ( {x}_{i} ) )}^{2} + λ {‖ θ ‖}_{2}^{2} \}$$

where μᵢ is the measured polarization and θ are the feature weights for input feature xᵢ of dimension d=2. It can be shown that the solution has the form, where α=(K+λI)⁻¹μ when rewriting θ in terms of a by the kernel trick.

The scikit-learn library was used to implement kernel ridge regression. We used an RBF kernel function, defined as K(xᵢ, xⱼ, s)=exp(−∥xᵢ−xⱼ∥²(2s²)⁻¹). The model's regularization parameter λ and length scale parameter s were tuned by the grid search algorithm. The best performing parameters were λ=1 and s=1. The optimized model's mean 5-fold cross validation RMSE was 0.0675 out of a response variable range of [0, 1].

### 1.5 Pattern Optimization

Based on the feature weights in our prediction model, we sought to find geometric arrangements that exhibit a high gradient over a length scale of 200 microns and a low density over a length scale of 700 microns. The trained kernel ridge regression model was used as a reward function for finding optimized patterns that maximize the minimum predicted polarization of a given set of organoid coordinates based on their location in feature space. The algorithms used to perform stochastic sampling of markers and optimization of patterns are described below. Additionally, we describe an algorithm to tile locally optimized patterns in a global grid.

### 1.5.1 Guided Sequential Sampling

Initializing random patterns globally, as was done to generate random data, led to slow convergence of optimization due to the small length scales of features chosen by 1.4.4. Thus, 1.5.1 was designed to generate locally dense random patterns for faster optimization. Initially, a single marker was uniformly randomly placed in a 2D array with side length 2000 pixels. Subsequently, k additional candidate coordinates in the 2D array were uniformly randomly sampled,

$$\{ {c}_{sim}^{1} , {c}_{sim}^{2} , … , {c}_{sim}^{k} \} ,$$

where k>1 is a hyperparameter. We computed the selected features ρ700 and ∥∇ρ200∥ for each coordinate and predicted the associated dipole moments \dot{μ} using the kernel ridge regression model. We selected the coordinate

$${c}_{sim}^{i}$$

that maximized the minimum polarization score among k coordinates and fixed it and only it to the 2D array. This process was repeated until N locally non-sparse coordinates were generated in the 2D array.

### 1.5.2 Simulated Annealing

For a given set of stochastically sampled markers generated by 1.5.1, we performed simulated annealing to optimize spatial coupling of organoid markers.

In our formulation of simulated annealing, at each step t, a coordinate is selected uniformly randomly and perturbed in a random direction, (δx, δy), with magnitude ∥(δx, δy)∥=m₀(r_{M})^{t} where m₀ is the initial magnitude and r_{M} is the decay rate of the magnitude. The random perturbation is accepted if either (i) the minimum predicted dipole moment \hat{ }μ improves relative to the previous step or (ii) p~Unif(0, 1)<p₀(r_{P})^{t} where p₀ is the initial probability and r_{P} is the decay rate of the acceptance probability. The chosen parameter values, m₀=80, r_{M}=0.9991, p₀=0.2, and r_{P}=0.9977, were optimized by grid search. This process was repeated for a total t=2000 steps.

Simulated annealing was performed sequentially on markers sampled by the method in 1.5.1, incrementing by one marker each time, until a local peak of the average minimum predicted polarization across 3 replicated was observed. The marker count of 6 resulted in the highest average minimum predicted polarization. All marker counts thereafter lead to a monotonic decrease in the average minimum predicted polarization. As a result, no more than 9 markers were tested. In total, simulated annealing was performed on 2 to 9 markers with 3 replicates each. Note that in the graph below norgs[n]_id[k] denotes the kth replicate of simulated annealing on n organoid markers.

### 1.5.3 Tiling

We sought to tile the hexagonal units from Section 1.5.2 in a global grid with the centroids of the hexagonal subunits fitting within the same dimensions as the random micropattern arrays (8000 by 8000). The spacing between hexagons were optimized in grid-search fashion over the range l∈{600, 650, . . . , 2100}, where 1 is the length between hexagon centroids. Given our global dimension constraints, this enabled us to generate a range of hexagonal arrays of grid size [8000/1+1]{4, . . . , 14}.

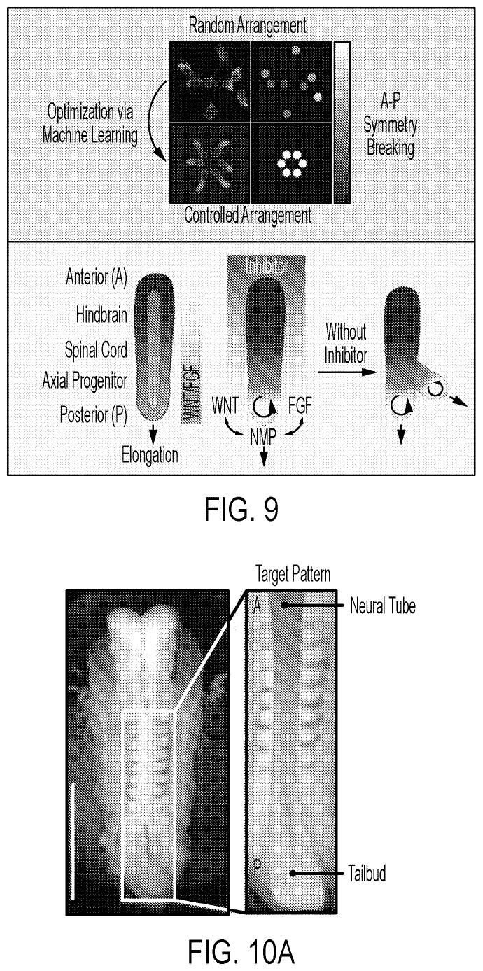

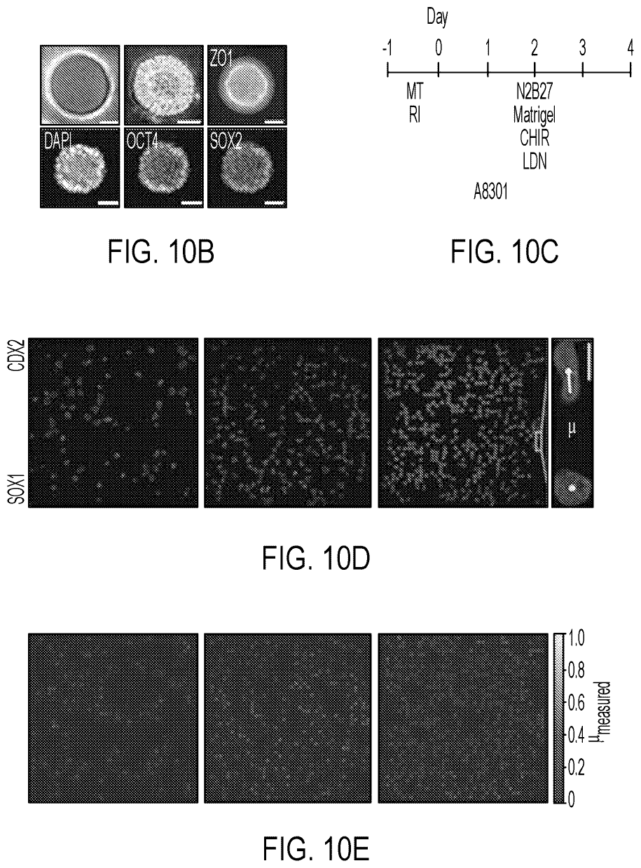

The predicted polarization for l={1300,1600,2000} (corresponding to grid sizes {7, 6, 5}) are shown in FIG. 7, along with the predicted polarization for the random array data (FIG. 8). We chose l=1600 with grid size 6 to minimize the distance between hexagons and thereby maximize the number of data points per array, while also maximizing minimum predicted polarization beyond any values predicted for random array data.

### Example 2. Controlling Organoid Symmetry Breaking Uncovers an Excitable System Underlying Human Axial Elongation

The human embryo breaks symmetry to form the anterior-posterior axis of the body. As the embryo elongates along this axis, progenitors in the tailbud give rise to tissues that generate spinal cord, skeleton, and musculature. This raises the question of how the embryo achieves axial elongation and patterning. While ethics necessitate in vitro studies, the variability of organoid systems has hindered mechanistic insights. Here we developed a bioengineering and machine learning framework that optimizes organoid symmetry breaking by tuning their spatial coupling. This framework enabled reproducible generation of axially elongating organoids, each possessing a tailbud and neural tube. We discovered that an excitable system composed of WNT/FGF signaling drives elongation by inducing a neuromesodermal progenitor-like signaling center. We discovered that instabilities in the excitable system are suppressed by secreted WNT inhibitors. Absence of these inhibitors led to ectopic tailbuds and branches. Our results identify mechanisms governing stable human axial elongation. See FIG. 8 and Anand G M et al. Cell. 2023 Feb. 2; 186(3): 497-512 (Anand 2023), the content of which (including supplemental materials) is incorporated herein by reference in its entirety.

During development, the human embryo elongates along its anterior-posterior (A-P) axis. Over the course of elongation, axial progenitor cells specified in the tailbud of the embryo give rise to the posterior neural tube and paraxial mesoderm. While the signals involved in A-P axis specification have been studied extensively, the roles and relationships of cell types and signals driving elongation are poorly understood, especially in humans. Furthermore, the mechanisms that govern stable elongation along a single axis are unknown.

Elucidating the mechanisms underlying human axial elongation requires the use of in vitro stem cell systems. In multiple instances, human and mouse pluripotent stem cell-derived organoids show spontaneous symmetry breaking when exposed to external signals and generate patterned tissues similar to those in developing mammalian embryos. However, the inherent variability in such in vitro systems makes it challenging to perform mechanistic studies. Overcoming such variability is therefore essential to achieve the goal of understanding human axial elongation.

Here, motivated by principles from physics, we developed an approach that combines bioengineering with machine learning to learn to build hundreds of organoids that reproducibly undergo deterministic A-P symmetry breaking and axial elongation. Using a combination of single-cell and in situ sequencing, live imaging, and perturbations, we characterized elongating organoids and uncovered distinct roles for FGF and WNT signaling during elongation. By determining conditions that sustain organoid elongation without external signals, we discovered that elongation is driven by an excitable system mediated by neuromesodermal progenitor (NMP)-like cells. We further identified a role for secreted inhibitors in maintaining the stability of this excitable system. Finally, we discuss the implications of our findings for modeling related morphogenetic processes and, more broadly, for building robust in vitro models of human development.

Spatial Coupling of Organoids Influences their Axial Patterning and Morphogenesis

In physical systems that undergo symmetry breaking, the underlying degrees of freedom can be coupled to reduce the entropy of the ground state. Furthermore, the desired ground state can be achieved in such systems by learning the correct couplings. For example, when N spins are not coupled to each other, and each spin can point in one of two directions, the entropy of the ground state is Nlog(2). In contrast, in an Ising ferromagnet, this entropy is log(2), because with ferromagnetic coupling, there are only two ground states: all spins point up or all point down. Inspired by these results, we asked whether we could couple organoids to increase the robustness of symmetry breaking and achieve the desired patterning and morphogenesis accompanying axial elongation in vivo. Specifically, we sought to build organoids that break A-P symmetry, specify a posterior tailbud and anterior neural tube, and elongate along the A-P axis (FIG. 10A). To test whether organoids could be coupled to each other, we cultured human pluripotent stem cells (hPSCs) on coverslips with randomly positioned 150-micron diameter micropatterns of extracellular matrix. Upon addition of matrix to the culture medium, hPSCs on each micropattern formed an epithelial cyst enclosing a single lumen, closely approximating the epiblast in vivo 15 (FIG. 10B). We obtained distinct random arrangements of epithelial cysts on each coverslip, each with a different average density (number of cysts per unit area). To generate organoids, we exposed these cysts to conditions that confer a posterior epiblast identity by activating the canonical WNT pathway using the GSK3B inhibitor CHIR99021 (CHIR) while inhibiting the BMP and TGFβ pathways using the SMAD inhibitors LDN193189 (LDN) and A8301 respectively (FIG. 10C). After four days of differentiation, we stained the differentiated organoids for CDX2, a posterior marker expressed by axial progenitors in the tailbud and SOX1, an anterior marker expressed by cells in the neural tube. Other pairs of markers could be used equivalently to assess A-P patterning, such as TBXT and SOX2. Under fixed differentiation conditions, the proportion of anterior SOX1+ versus posterior CDX2+ fates on a coverslip was positively correlated with average organoid density (r=0.98) (FIG. 10D).

Furthermore, organoids with polarized expression of SOX1 and CDX2 tended to elongate along the SOX1-CDX2 axis, while unpolarized organoids did not elongate (FIG. 10D, right). To quantify the variability in polarization across organoids, we defined polarization as the distance between the centroid of CDX2+ cells and the centroid of the organoid (see Example 1). We generally observed significant variations in polarization and axial elongation on individual coverslips. However, in replicate experiments with identical initial arrangements, organoids at the same micropattern positions showed lower variability in polarization and elongation (data not shown). We concluded that organoids could be coupled and influence each other's patterning and morphogenesis based on their relative spatial arrangement.

To test whether secreted factors in the extracellular media mediated organoid coupling, we transferred media conditioned by a high-density array to a low-density array (see Examples 1 and 2). Organoids in the low-density array exhibited a shift toward a higher proportion of SOX1+ cells and a lower proportion of CDX2+ cells, suggesting that factors secreted into the culture media are at least partly responsible for mediating organoid coupling by promoting an anterior neural fate at the expense of a posterior axial progenitor fate (data not shown).

### Machine Learning-Driven Optimization of Organoid Arrangements Enables Reproducible Axial Elongation

To systematically learn how to spatially arrange organoids to obtain the desired patterning and morphogenesis accompanying axial elongation, we used approaches from machine learning (see Example 1). We first measured the polarization of CDX2 expression μimeasured for each organoid i in all 2373 organoids across our random micropattern experiments (FIG. 10E). We then calculated a multi-dimensional feature vector for each organoid, v→i, with components consisting of the organoid density ρⁱ and the gradient of organoid density |∇ρⁱ| over a range of length scales from 100 microns to 2000 microns, i.e., v→i=(ρi100, . . . ,ρi2000,|∇ρi100|, . . . ,|∇ρi2000|) (FIG. 10F). We sought to learn the function ƒ of the feature vector that predicted the polarization of each organoid i, i.e., μipred=f(v→i), with the minimum least squares error, Σi(μipred−μimeasured)2.

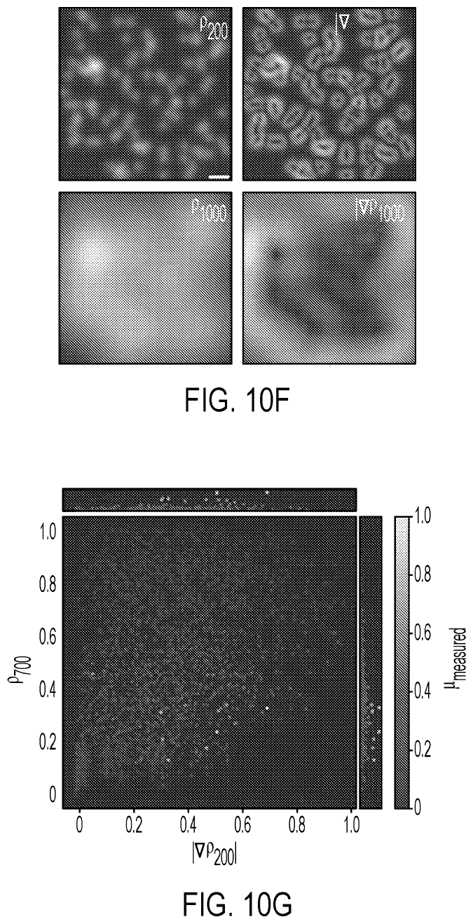

We used sequential forward selection to determine the most predictive subset of features in v→, followed by kernel ridge regression with a radial basis function kernel and five-fold cross-validation to learn the parameters of ƒ. We determined that two components of v→i were most predictive of organoid polarization: the organoid density at 700 microns, ρi700, and the gradient of organoid density at 200 microns, |∇ρi200| (FIG. 10G). Using the learned function ƒ, we mapped out the predicted polarization of organoids in the space of these two components (FIG. 10H). We then performed simulations to modify the arrangements of organoids until each organoid was predicted to have maximum polarization. To do so, we first performed simulated annealing on small numbers of organoids ranging from 2 to 9 and found that 6 organoids arranged in a hexagon achieved the maximum predicted polarization (FIGS. 10I and 9J). To determine the arrangement of these hexagonal units on a coverslip, we calculated μ_{pred} for each organoid as a function of the distance between these units. We selected the distance such that the predicted polarization was uniformly high across all organoids while fitting as many organoids as possible on a single coverslip (FIG. 10K). The computationally selected arrangement thus consisted of a 6 by 6 array of hexagonal units spaced 1.6 mm apart (FIG. 10L). We conducted our differentiation experiments on coverslips micropatterned with this hexagonal array. After 4 days, every organoid in the array that initially formed a cyst within a hexagonal unit developed a CDX2+ tailbud domain, a SOX1+ neural tube domain, and elongated axially (n=204) (FIG. 10M). Thus, we were able to experimentally tune the coupling between organoids by optimizing their spatial arrangement and inducing them to jointly break A-P symmetry to develop into our desired target patterned tissue.

To understand why hexagonal arrangements of organoids are effective at coherently breaking symmetry, we built a mathematical model of the spatial coupling between organoids via a secreted, diffusive signal that promotes the anterior fate and inhibits the posterior fate under external WNT activation (see Examples 1 and 2). The model demonstrated that in hexagonally patterned organoids with a close center-to-center spacing, the signal level was enriched at the interior-facing side of each organoid. Thus, the regions of the organoids facing inwards adopted an anterior fate, while the regions facing outwards adopted posterior fates (FIG. 10O, left). When the organoids in the mathematical model were moved farther apart beyond a critical spacing, the coupling between them through the diffusive signal decreased, and organoids failed to break symmetry robustly (FIG. 10O, right). Consistently in experiments, increasing the spacing between organoids on the hexagon from 200 microns to 400 microns led to a loss of coupling and led to variable polarization of individual organoids (data not shown).

We found that the correctly patterned organoids recapitulated several aspects of the elongating neural tube in vivo. Elongating organoids contained a single lumen enclosed by apical tight junctions (marked by ZO1), a basement membrane (marked by laminin). They exhibited apical mitosis (marked by phospho-histone H3) along the whole A-P axis, consistent with interkinetic nuclear migration (FIG. 10N). The posterior tailbud region marked by CDX2 was composed of a single-layer epithelium (data not shown), consistent with observations in mammalian embryos of the chordoneural hinge and its precursor populations, the node-streak border and caudal lateral epiblast, which collectively house axial progenitors in vivo. We also observed pseudostratified SOX2+ nuclei as well as junctional N-cadherin and apical F-actin (data not shown). Upon staining organoids for CDX2 and SOX1 at sequential timepoints, we found that CDX2+ axial progenitors were synchronously specified at the prospective posterior pole by day 2 of differentiation and maintained throughout elongation, and a SOX1+ neural tube was specified anteriorly that increased in length over time (FIG. 10P, top). The mean A-P axis length of organoids increased from approximately 150 microns on day 1 to approximately 400 microns on day 4 (FIG. 10P, bottom) and continued to increase through day 7 (see Examples 1 and 2), by which time organoids reached lengths over 1 mm (FIG. 10P, right).

### Elongating Organoids Contain Hindbrain, Spinal Cord, and Axial Progenitors

To identify the cell types present in elongating organoids, we used single-cell RNA sequencing (scRNA-seq). We performed scRNA-seq on cells pooled from 12 organoids on day 4 of differentiation to obtain high-quality transcriptomes of 7752 cells. Using unsupervised sparse multimodal decomposition (SMD) to infer key genes and cell types simultaneously, we identified a subspace of 26 genes in which we could cluster cells into 4 progenitor cell types, corresponding to spatially distinct regions along the A-P axis. The observed cell types included axial progenitors (marked by CDX2) as well as progenitors of the cervical spinal cord (marked by HOXB4 and CRABP1), posterior hindbrain (marked by NR2F1), and anterior hindbrain (marked by EGR2). We also observed a small cluster of neuronal cells (marked by HES6 and BTG2). Interestingly, we did not observe any transcriptional signatures of neuromesodermal progenitors (NMPs, marked by TBXT and SOX2), paraxial mesoderm derivatives (marked by TBX6 and MEOX1), or lateral mesoderm derivatives (marked by FOXF1), suggesting that elongation can occur even in the absence of these cell types under external WNT activation. We further demonstrated that the axial progenitors in these organoids are transcriptionally distinct from NMPs and closer to pre-neural tube (PNT) progenitors, despite both cell types existing in the mammalian tailbud and its precursor tissues in vivo. This is in concordance with the idea that the embryonic tailbud consists of multiple axial progenitor cell types. See also Anand 2023.

### WNT and FGF Signals are Posteriorly Polarized in Elongating Organoids

Having identified the constituent cell types, we proceeded to investigate the underlying signals that initiate and drive axial elongation. An unbiased search for differentially expressed transcription factors, receptors, and secreted factors across progenitor clusters identified several known WNT and FGF ligands, receptors, and downstream targets. We therefore performed a global single-cell analysis of WNT and FGF pathway genes. We also catalogued the corresponding loss of function axial elongation phenotypes in mouse mutants. To infer spatial profiles of these signaling pathways in organoids, we built a diffusion map in the Euclidean space of genes identified by SMD to order cells from scRNA-seq along a putative posterior-anterior (P-A) axis. We first validated this pseudo-spatial ordering by verifying the expected P-A localization of axial progenitor, spinal cord, and hindbrain transcription factors such as CDX2, MEIS2, HOXB4, and HOXB1. We then used this pseudo-spatial P-A axis to determine profiles of signaling pathways implicated in axis formation. Out of all WNT ligands, only noncanonical WNT ligands WNT5A and WNT5B were posteriorly polarized. We also observed posterior expression of noncanonical WNT component PRICKLE1 and canonical WNT component LEF1. We observed globally high expression of secreted WNT inhibitors SFRP1 and SFRP2, and anterior expression of FRZB. We did not detect the expression of canonical WNT inhibitors DKK1 or CER1. We observed posteriorly polarized expression of FGF ligands FGF2, FGF8, FGF12, and FGF17, anteriorly polarized expression of FGF3, and posterior expression of FGF/ERK targets SPRY1, SPRY4, DUSP6, and IL17RD (SEF) as well as PEA3-family transcriptional effectors ETV4 and ETV5. To validate these signaling profiles, we performed in situ sequencing on day 4 organoids using STARmap. We implemented a revised STARmap protocol and corresponding computational pipeline compatible with 3D tissue sequencing and analysis to spatially map 16 genes selected from scRNA-seq data. Across three replicates, we observed posteriorly polarized expression of CDX2, WNT5A, PRICKLE1, and FGF8, and global expression of SFRP1 and SFRP2, matching our inferences from scRNA-seq data. The results from in situ sequencing validated the cell types and diffusion map trajectories inferred from scRNA-seq. Our analyses thus demonstrate that WNT and FGF/ERK signals are localized posteriorly to the tailbud of axially elongating organoids. See also Anand 2023.

### WNT Ligands Drive Axial Elongation Downstream of Canonical WNT Activity

Given that WNT signals are polarized in organoids, we next asked whether WNT ligands drive axial elongation downstream of CHIR-mediated canonical WNT activation. We inhibited WNT ligand activity using the Porcupine inhibitor IWP3, which blocks WNT secretion by preventing its palmitoylation. We found that IWP3-treated organoids failed to elongate and retained a spherical shape after four days of differentiation. However, IWP3-treated organoids retained a significant posterior CDX2+ population, similar in size to control organoids when adjusted for A-P length. This suggested that secreted WNT ligands are necessary for elongation in the presence of CHIR, but not for the initial specification of posterior identity. To test whether secreted WNT ligands were required continuously for elongation or acted as an initial trigger, we treated elongating organoids on day 2 with IWP3. We found that organoids were truncated by day 4 despite possessing CDX2+ domains, demonstrating that continuous secretion of WNT ligands was necessary for elongation.

To explore the role of WNT ligands in regulating cell behaviors during elongation, we performed live confocal time-lapse imaging of organoids expressing ZO1-EGFP, a fusion reporter of apical tight junctions, with or without delayed IWP3 treatment on day 2 of differentiation. We verified that IWP3 treatment led to truncation of ZO1-EGFP+ organoids even in the presence of a posterior CDX2+ domain. By tracking the displacement of individual cell clones over the course of fourteen hours, we found that neighboring clones in control organoids exhibited higher mean squared relative displacements compared to IWP3-treated organoids. This suggested that cells experienced increased fluidity in the presence of WNT ligands. We also found that control organoids displayed directed cell movements in the plane of the epithelium along the direction of the elongation axis. In contrast, cell movements in IWP3-treated organoids were undirected with respect to the elongation axis. We additionally performed time-lapse imaging of organoids expressing H2B-mCherry under control conditions to determine whether cell divisions were oriented with respect to the elongation axis. However, there was no correlation between the angle of cell division and elongation direction. We concluded that WNT ligands are continuously required to maintain the fluidity of the epithelium to facilitate the rearrangements of neighboring cells in the direction of the elongating axis. Furthermore, the detection of no posterior canonical WNT ligands in single-cell expression data and the observation that the addition of DKK1, a canonical WNT receptor inhibitor, does not affect elongation, suggests that secreted canonical WNT ligands do not play a direct role in elongation. These observations support a potential role for noncanonical WNT ligands in driving elongation. See also Anand 2023.

### FGF/ERK-Dependent CDX2 Expression is Required for Elongation

We next tested the roles of intrinsic and extrinsic signaling factors implicated upstream of WNT ligand activity in the tailbud. Across vertebrates, WNT ligands are induced by CDX transcription factors. We used CRISPR interference (CRISPRi) to knock down CDX2 expression in a constitutive CRISPRi hPSC line. We generated organoids from CRISPRi lines with an integrated CDX2 guide RNA (gRNA) or nonspecific control gRNA. By day 4, organoids with a control gRNA elongated normally. In contrast, organoids with a CDX2 gRNA were truncated and lacked CDX2+ domains, suggesting that CDX2 is essential for organoid elongation. Truncation was accompanied by a decrease in proliferation in the tailbud region, based on a reduction in phospho-histone H3 staining, as well as a loss of posterior WNT5A and WNT5B gene expression, based on in situ hybridization chain reaction. To determine if a WNT5A/B knockdown resembles the CDX2 knockdown, we generated CRISPRi organoids with an integrated WNT5A gRNA and transduced them with a virus carrying a WNT5B gRNA, achieving greater than 50% efficiency of genomic integration. Organoids lacking WNT5A alone elongated similarly to wild-type organoids, while organoids lacking both WNT5A and WNT5B were partially but significantly truncated. These results, in conjunction with earlier observations, suggest that noncanonical WNT ligands WNT5A/WNT5B play a role in driving elongation downstream of CDX2.

CDX transcription factors are thought to act downstream of FGF/ERK and canonical WNT signaling in vertebrates. To test the role of FGF/ERK, we treated organoids with PD0325901 (PD0), a small molecule inhibitor of ERK1/2 phosphorylation. Following continuous FGF/ERK inhibition, organoids failed to elongate or produce any CDX2+ axial progenitors. This suggested that FGF/ERK was necessary to induce CDX2 expression and subsequent axis elongation. To determine whether FGF/ERK signaling was required throughout the elongation process or only at the outset, we inhibited FGF/ERK on day 2 of differentiation. By day 4, organoids were truncated relative to control organoids and downregulated CDX2 expression, showing FGF/ERK was required not only to induce CDX2 but also to maintain its expression level for elongation to occur. Our results support a model in which canonical WNT activity (driven by CHIR) together with FGF/ERK activity (driven by endogenous FGF ligands) induces and maintains CDX2 expression in the tailbud. In turn, CDX2 drives the secretion of WNT ligands, which are essential for directing elongation by modulating cellular rearrangements. See also Anand 2023.

### Elongation is Sustained by the Induction of a Neuromesodermal Progenitor-Like Signaling Center

In experiments described thus far, we required sustained canonical WNT activation through CHIR to drive elongation. We next asked if organoids had the ability to elongate autonomously even in the absence of this external drive. Axial elongation is thought to be sustained in vivo by NMPs via the production of WNT and FGF ligands. Studies in vertebrates have further demonstrated that canonical WNT and FGF/ERK pathways are engaged in a positive feedback loop in a variety of developmental contexts, including the zebrafish and mouse tailbud during somitogenesis. We sought to exploit this feedback between WNT and FGF activity to sustain elongation. Thus, we exposed organoids to a transient 24-hour pulse of recombinant FGF2 prior to withdrawing CHIR on day 2 of differentiation. Under control conditions, CHIR withdrawal led to truncated organoids with reduced CDX2 expression, whereas FGF pulse-treated organoids continued to elongate through day 4 of differentiation, showing that a pulse of FGF was sufficient to sustain elongation in the absence of CHIR. To verify the induction of NMPs, we stained FGF pulse-treated organoids for TBXT and SOX2, transcription factors which are known to jointly mark NMPs in vivo. Cells in the tailbud of FGF-treated organoids stained positively at the end of the pulse for TBXT and SOX2, with SOX2 at lower levels than in the neural tube, consistent with in vivo expression patterns. In contrast, control organoids exposed to CHIR alone failed to express TBXT. Furthermore, FGF pulse-treated organoids maintained TBXT+SOX2+ cells in the CDX2+ tailbud region over the course of elongation, although these cells decreased in number by Day 4. We also found that a CHIR pulse at a concentration higher than in control conditions could generate a similar result in an FGF/ERK-dependent manner. In vertebrates, WNT and FGF ligand production in NMPs is thought to be driven in part by the transcription factor TBXT. To test the requirement of TBXT for sustained elongation, we generated CRISPRi organoids with an integrated TBXT guide RNA and exposed them to continuous CHIR or FGF pulse conditions. Compared to CRISPRi organoids containing a control gRNA, organoids with a TBXT gRNA were truncated following the FGF pulse. This is consistent with the possibility that TBXT is required for the production of canonical WNT and FGF/ERK signals by NMPs during axial elongation. Accordingly, TBXT+SOX2+ cells from organoids generated via a CHIR pulse express canonical WNT ligand WNT3A and as well as high levels of FGF ligands FGF8 and FGF17, which activate canonical WNT and FGF/ERK pathways, respectively. We observed similar signaling profiles in NMPs derived from the posterior mammalian epiblast in vivo. Thus, induction of an NMP-like signaling center enables self-sustained axial elongation of neural tube organoids, likely through the continuous production of posterior signals. See also Anand 2023.

### Secreted WNT Inhibitors are Required for Stable Axial Elongation

These results suggest that organoids can be excited from a state in which elongation relies on external WNT signals to one in which it can be self-sustained by positive feedback of endogenous FGF and WNT signaling, following an FGF or CHIR pulse. Such excitable systems that exhibit positive feedback are known to be susceptible to runaway events. A simple mathematical model of the organoid with positive feedback leads to self-sustained activity of an activator (e.g., FGF or WNT) at the tailbud. In the presence of a diffusible inhibitor, the effects of transient ectopic activation away from the tailbud are suppressed. In contrast, when the inhibitor is removed, ectopic activation triggers persistent activity through the positive feedback loop. Such persistent activity could lead to runaway events such as ectopic axial elongation.

Therefore, we asked whether diffusible WNT or FGF inhibitors might constrain these positive feedback loops to ensure stable elongation along the A-P axis. In our scRNA-seq and STARmap data, we observed globally high expression levels of secreted WNT inhibitors SFRP1 and SFRP2. To verify that organoids were secreting SFRPs, we stained for SFRP1 protein on day 4. SFRP1 protein was distributed uniformly across the surface of organoids. We further adjusted the contrast to visualize SFRP1 protein bound to the coverslip, which showed that SFRP1 was distributed in a gradient along the radial direction away from the center of each hexagon. This gradient of SFRP1 protein on the coverslip was not an artifact, as it disappeared upon knockdown of SFRP1. To test whether SFRP1 and SFRP2 suppressed runaway events leading to ectopic sites of elongation, we generated organoids from CRISPRi lines with an integrated SFRP2 gRNA, and treated organoids with WAY316606 (WAY3), a small molecule that inhibits SFRP1 activity by preventing its interaction with WNT ligands. While knockdown of SFRP2 or inhibition of SFRP1 alone did not produce severe defects, simultaneous SFRP1 inhibition and SFRP2 knockdown resulted in ectopic tailbud formation and elongation, characterized by branched structures and an increased average number of tailbuds per organoid. Branched structures were not correlated with changes in proliferation rates in the tailbud, suggesting branching may occur due to a defect in the cellular rearrangements underlying elongation. We also generated CRISPRi organoids with dually integrated SFRP1 and SFRP2 gRNAs. These organoids exhibited similar defects, and additionally and did not generate an extracellular gradient of SFRP1. Thus, redundantly acting secreted WNT inhibitors in the form of SFRPs are required to ensure that elongation occurs stably along a single axis. See also Anand 2023.

### DISCUSSION

Stochasticity has attracted much attention in biological systems. The large variability exhibited by in vitro organoid models is often attributed to such intrinsic noise. In contrast, it appears that much of the variability observed in vitro is due to variability in spatiotemporal signaling conditions and geometry. Our study shows that this variability can largely be eliminated if the boundary conditions for the underlying dynamical systems can be controlled, in this case, by coupling these systems to each other. Specifically, we built a bioengineering and machine learning framework to learn how to couple organoids to one another and control organoid symmetry breaking in a deterministic manner. Learning to reduce variability in biological systems using a combination of engineering and statistical inference methods has the potential to aid in the discovery of mechanisms underlying the development of complex tissues (e.g., human tissues).

Our reproducible organoid system enabled us to uncover mechanisms underlying human axial elongation. Our findings show that axial elongation is driven by posteriorly secreted FGF and WNT ligands. The critical constituent cell type secreting these signals in the tailbud is an NMP-like population characterized by SOX2 and TBXT expression that acts as a posterior signaling center in the absence of exogenous signals. The NMP-like cells, together with the secreted WNT and FGF signals, form a positive feedback loop to drive autonomous elongation. Despite being bipotent, NMP-like cells give rise only to neural progenitors under the conditions described in this manuscript. This suggests that their ability to act as a signaling center is independent of their potency to generate mesodermal tissue. Accordingly, organoids consist only of a neural tube and tailbud and can elongate axially in the absence of any mesoderm.

Furthermore, as with all excitable systems, including those in biology, elongating organoids are prone to runaway events triggered by a positive feedback loop, which is composed of FGF and WNT signals. Removing inhibitors of these signals reveals the instability of the underlying dynamical system, leading to the emergence of ectopic sites of elongation. We propose that secreted inhibitors of the WNT pathway in the form of SFRPs are required for stable elongation along a single anterior-posterior axis.

Our model of the elongating neural tube as an excitable system could generalize to other organ primordia in which asymmetric signals are coupled to morphogenesis, especially those in which WNT and FGF signaling have essential roles. These include the limb bud and lung bud, where the polarization of gene expression domains and elongation are essential for proper development. In these instances, too, the identification and knockdown of key inhibitors could uncover instabilities of the underlying dynamical system that drives the outgrowth of these tissues.

Furthermore, the ability of our organoids to robustly generate a posterior population of NMP-like cells opens avenues to studying the coordination of human spinal cord and musculoskeletal development in vitro, as we show in a companion manuscript. While the neural tube can elongate in the absence of paraxial mesoderm, the mesoderm-derived somites serve as an important source of retinoic acid, which mediates downstream patterning of the neural tube by the induction of neural genes such as PAX6. By systematically building up the complexity of tissue-tissue interactions present in the embryo, we may uncover developmental mechanisms that are difficult to dissect in vivo.

### EXPERIMENTAL MODELS AND SUBJECT DETAILS

### Cell Lines

We conducted the majority of our experiments using WA01 human ESCs (H1, WiCell). For timelapse studies, we used WTC-11 human iPSCs (WTC, Allen Institute) with endogenous ZO1 tagged with mEGFP (AICS-0023) as well as hiPSCs with H2B tagged with mCherry (H2B-mCherry) introduced at the AAVS1 locus (provided generously by the Pourquie Lab⁴⁵). For CRISPRi studies, we used WTC-11 hiPSCs with dCas9-TagBFP-KRAB introduced at the CLYBL locus (AICS-0090). hPSCs were cultured in 6-well tissue culture dishes treated for 1 hour with 1× diluted Matrigel (Corning 354277) and supplied with mTeSR Plus media (Stem Cell Technologies 5825). For routine culture, we passaged by washing with phosphate buffered saline (PBS) followed by ReLeSR (Stem Cell Technologies 5872) treatment for 5 minutes. Every 5 days, cells were passaged in clumps of 10 to 20 cells and seeded in mTeSR Plus. All cell lines used were routinely tested for mycoplasma contamination (Mycoplasma PCR Detection Kit, ABM G238).

For lentiviral production, we used the Lenti-X™ 293T Cell Line (Takara 632180), a subclone of the HEK 293 cell line. Cells were split 1:10 every 3 days using 0.25% Trypsin-EDTA into a tissue culture treated 10-cm dish (Thermo Fisher 150318) and maintained in 10% fetal bovine serum (FBS) in DMEM/F12 (Thermo Fisher 11039021).

For bacterial transformation, we used NEB Stable Competent E. coli (NEB C3040H). Prior to use, cells were thawed on ice for 10 minutes.

### Method Details

### Soft Lithography and Microcontact Printing

Microcontact printing was performed using polydimethylsiloxane (PDMS) stamps cast on soft lithography masters fabricated in a non-cleanroom setting. Briefly, round ADEX-50 dry film photoresist films (DJ Microlaminates) were laminated onto round 76.2 mm Si wafers (University Wafers) at 65° C. using a SKY laminator. Films were exposed through 8 μm-resolution photomasks (CAD/Art Services), designed on AutoCAD, to 365 nm UV light at an intensity of 25 mW/cm\hat{ }2 for 13 seconds using a UV lamp (Uvitron), baked at 80° C. for 15 minutes, and developed face down in cyclohexanone (VWR BDH4612) on a steel mesh without shaking for 5 minutes, followed by washing with acetone and isopropyl alcohol and drying with compressed air. Afterwards, masters were hard-baked on a hot plate at 200° C. for 1 hour and silanized for 1 hour in a vacuum chamber with 50 μl of trichloro(1H,1H,2H,2H-perfluorooctyl) silane (Sigma 448931).

To cast PDMS, the elastomer base and curing agent (Dow Sylgard™ 184, Ellsworth Adhesives) were mixed at a ratio of 1:9, degassed, poured onto masters and baked at 80° C. for 1 hour. Stamps were covered with Scotch tape to prevent dust accumulation and cut using a scalpel. Stamps were brought into the biosafety hood and coated overnight with extracellular matrix consisting of 1× diluted Matrigel, washed with MilliQ water, air dried, and placed onto 22 mm No. 1.5 square borosilicate glass coverslips (VWR 48366-227). Stamps were removed with tweezers just prior to seeding cells.

### Organoid Differentiation

hPSCs were seeded onto microcontact-printed coverslips containing 150-μm diameter features at 1-2 million cells/mL in pluripotency maintenance media consisting of mTeSR Plus media (MT) supplemented with 10 μM ROCK inhibitor Y-27632 (RI) and washed with fresh MT+RI after 1-2 hours. Once colonies were confluent (day 0), media was switched to differentiation media. Differentiation media consisted of (DMEM/F12 with 1×N2, 1×B27, 1× penicillin/streptomycin, 1% NEAA, 0.5% GlutaMAX, 0.1% ß-mercaptoethanol, and 0.05% bovine serum albumin) supplemented with 0.5 μM A8301, 0.5 μM LDN193189 (LDN), 3 μM CHIR99021 (CHIR), and 6% v/v Matrigel. After 2 days, media was replaced with N2B27 media supplemented with 0.5 μM LDN and 3 μM CHIR. On hexagonal arrays, organoids began to elongate by day 2 and continued to elongate through day 4. We note that the elongating portion of the organoid was surrounded by extracellular matrix throughout these 4 days, rather than being attached to the glass surface, as indicated by movement upon gentle shaking of the culture dish. For long-term culture to day 7, organoids were removed from coverslips and transferred to a 96-well plate. Notably, LDN was not required for the prevention of paraxial mesoderm induction from day 2 onwards. For conditioned media experiments, media on a random low-density array was replaced on day 1 and day 3 from a random high-density array. For pulse experiments, media was supplemented on day 1 with an additional 1 μM CHIR99021 or 20 μg/ml recombinant human basic fibroblast growth factor (FGF2) for 24 hours, then washed and replaced with N2B27 media with 0.5 μM LDN. For perturbation experiments, media was supplemented on the days indicated in the text with 1 μM IWP3, 1 μM PD0325901 (PD0), or 20 μM WAY316606 (WAY3).

### Immunostaining

Cells were fixed with 4% formaldehyde for 15 minutes at room temperature. Fixed cells were treated with blocking buffer (PBS+5% normal donkey serum+0.3% Triton X-100) for 1 h, then overnight at 4° C. with primary antibody diluted in staining buffer (PBS+1% BSA+0.3% Triton X-100). The following primary antibodies were used at the indicated dilutions: Rabbit anti-OCT4 (1:400, Cell Signaling C30A3); Rat anti-SOX2 (1:400, Thermo Fisher BTJCE), Rabbit anti-N-Cadherin (1:400, Cell Signaling D4R1H), Mouse anti-ZO1-FITC (1:800, Thermo Fisher 1A12), Rabbit anti-CDX2 (1:400, Cell Signaling D11D10), Mouse anti-CDX2 (1:200, R & D MAB3665), Goat anti-SOX1 (1:400, R & D AF3369), Mouse anti-SOX1 (1:200, BD Biosciences N23-844), Mouse anti-LAMC1 (1:50, DSHB 2E8), Rabbit anti-pHH3 (1:400, Cell Signaling 9701), Goat anti-TBXT (1:400, R & D AF2085), Goat anti-TBX6 (1:400, R & D AF4744), Goat anti-SFRP1 (1:200, R & D AF1384), Alexa Fluor™ 647 Phalloidin (1:20, Thermo Fisher A22287).

After overnight incubation with primary antibody solution on an orbital shaker at 100 rpm, samples were washed three times with PBS, then secondary antibodies diluted in staining buffer were added. We used the following secondary antibodies all at a dilution of 1:1000: donkey anti-rabbit Alexa 568 (Thermo Fisher), donkey anti-goat 488, donkey anti-rat Alexa 488 (Thermo Fisher), and donkey anti-mouse Alexa 647 (Thermo Fisher). We added 300 nM DAPI (Thermo Fisher) to the secondary antibody staining solution to visualize nuclei. After overnight incubation with secondary antibody solution on an orbital shaker at 100 rpm, samples were washed three times with PBS and stored at 4° C. for imaging.

Epifluorescence Imaging of Fixed Samples Fixed samples were imaged on a Zeiss AxioObserver Z1 inverted microscope using Zeiss 10× and 20× plan apo objectives (NA 1.3) using the appropriate filter sets. Images were acquired using an Orca-Flash 4.0 CMOS camera (Hamamatsu). The 43 HE DsRed/46 HE YFP/47 HE CFP/49 DAPI/50 Cy5 filter sets from Zeiss were used. The microscope was controlled using ZEN software.

### Confocal Imaging of Fixed and Live Samples

Fixed samples were imaged on a Zeiss LSM 980 with Airyscan using a Zeiss 10× (NA 0.45) objective. Detection was performed on DAPI, AlexaFluor 488, AlexaFluor 568 and AlexaFluor 647 channels. A Z-stack with 1-micron intervals was acquired from the lower to upper apical surface of each organoid. After Airyscan processing, a maximum Z intensity projection was performed for visualization purposes.

For live imaging, organoids were maintained in a 37° C. incubation chamber at 5% CO₂ and imaged using a Zeiss 20×(NA 0.8) objective every 20 minutes. Detection of ZO1-EGFP was performed on the AlexaFluor 488 channel using the 488 nm laser at 3% intensity, and detection of H2B-mCherry was performed on the AlexaFluor 568 channel using the 561 nm laser at 5% intensity. Focus was maintained using Zeiss Definite Focus to compensate for sample drift. For maximum accuracy, cells in ZO1 and H2B time lapses were tracked manually in FIJI. ZO1 tracks were analyzed with a custom MATLAB script to track net and mean squared relative displacements.

### Analysis of Live Confocal Time Lapses

Three dimensional images of ZO1 time lapses were compressed to two dimensions+time using a custom MATLAB script. This script applied a threshold to the image based on the ZO1 intensity of each pixel, extracting the pixels with high ZO1 intensity. A plane was fitted to this point cloud and the 2D image was extracted from taking the average of 5 pixels bellow and above the plane. This was done to extract only the bottom apical surface of the organoids, since the top surface also has fluorescence intensity, and this could produce noise in a maximum intensity projection. The ZO1 projection was then analyzed using the Manual Tracking plugin on FIJI. The tracks were later exported to MATLAB and for each track we calculated the displacement vectors of the position of the cell center between the initial and final time point that the cell was tracked. We calculated the angle between these vectors and the elongation axis of the organoid defined with respect to the center of the organoid. To calculate the mean squared relative displacement (MSRD) we detected the closest neighbor to each cell at the beginning of that cell's track and measured the distance between these neighbors across 10 time points. The initial distance was subtracted from the measurements to obtain relative distances. The relative distances were squared, and the average squared relative distance for each time point was taken as the MSRD for that time point.

The H2B time lapses were transformed to two dimensional+time images using a simple maximum intensity projection on FIJI. Using the H2B timelapse projection we analyzed the cell division orientation. Cell divisions were identified manually by the characteristic shape and brightness of the condensing chromosomes. We labeled a position in between daughter cells representing the closest position to the mother's nuclei in the previous time point together with the centers of the daughter cells. Vectors were drawn from the mother to the daughters, and we calculated the angle of those vectors to the axis of elongation using a custom MATLAB script.

### Image Segmentation

All epifluorescence images were segmented in FIJI or Ilastik and analyzed in MATLAB or Python. Confocal images of pHH3 stains were segmented at single cell resolution in Arivis Vision4D using a machine learning pipeline and analyzed in Python.

### Single Cell RNA Sequencing

Organoids were dissociated into a single-cell suspension using the Worthington Papain Dissociation System kit (Worthington Biochemical). Cells were counted on the LUNA-FX7 Automated Cell Counter (Logos Biosystems) using fluorescence detection for viability with an acridine orange/propidium iodide stain. The sample was 98.3% viable and had a concentration of ~1000 cells/ul. After counting, the sample was loaded into Chip G per the user guide from 10× Genomics, and no alterations were made at any step of the protocol. GEMs were formed targeting 10,000 cells and reverse transcription completed immediately after. The cDNA was cleaned from the GEM reagents, amplified for a total of 11 cycles and verified via TapeStation (Agilent Technologies). Amplified cDNA was diluted and ran on the 4200 TapeStation instrument using High Sensitivity D5,000 tape and reagents (Cat #5067-5592 & 5067-5593). The amplified cDNA was fragmented, end repaired, and A-tailed followed by adaptor ligation, and PCR amplification for a total of 11 cycles with each sample receiving a unique set of dual indices (Cat #1000215). Final libraries were diluted and ran using the High Sensitivity D5,000 tape and reagents (Cat #5067-5592 & 5067-5593) on the 4200 TapeStation (Agilent Technologies). Libraries were quantified via Kapa qPCR using the Complete Universal Kit (Cat #07960140001, Roche Sequencing Solutions) and the CFX96 Touch Real-Time PCR Detection System (Bio-Rad Laboratories). Libraries were sequenced on an Illumina NovaSeq instrument using the parameters outlined in the user guide (Read1: 28 bp, i7 index: 10 bp, i5 index: 10 bp, Read2: 90 bp). After sequencing and demultiplexing, the Cell Ranger count pipeline was used to align reads to the GRCh38 human reference genome and produce the associated cell by gene count matrix.

Cells with 5,000 to 50,000 reads were subsampled to a obtain a read count of 5,000 for each cell. Sparse multimodal decomposition (SMD) was performed in Python on the subsampled count matrix. Genes with a Z score from SMD greater than 0 were filtered to remove genes with a log normalized expression mean or standard deviation less than 0.02, genes expressed in more than 90% or less than 10% of cells, and genes associated with the cell cycle, resulting in a list of 26 genes.

To cluster cells in this 26-gene space, we scaled genes to have unit variance and zero mean, then performed Louvain clustering at a resolution of 0.75 using scanpy. We obtained 5 clusters, which were annotated manually based on expressed genes. Diffusion map analysis was performed using the diffusion pseudotime (DPT) method in scanpy. The root cell was selected to be a cell from the axial progenitor cluster with the highest value along the first principal component from principal component analysis. To generate a pseudo-spatial gene expression heatmap, cells were ordered by pseudo-spatial index and their raw gene expression values were smoothed using a Gaussian filter with a kernel size of 300 cells. Genes were then ordered by the center of mass of smoothed gene expression values. To generate pseudo-spatial gene expression plots for individual genes, raw gene expression values were binned into 20 bins of equal size and their mean and standard deviation within each bin was plotted.

To generate a list of transcription factors, receptors, and secreted factors enriched posteriorly and anteriorly, respectively, genes from the corresponding protein classes of the human protein atlas were filtered to keep only those genes whose expression in each progenitor cluster was at least 1.5-fold larger than the next most anterior or posterior cluster. To combine mouse and human data, following pre-processing of each dataset, Louvain clustering at a resolution of 0.8 was performed in scanpy in the space of the concatenated list of posterior and anterior transcription factors across all 4 progenitor clusters, after first filtering out genes that were species-specific.

### Mapping In Vitro and In Vivo Single Cell Data

To compare our day 4 organoid data against an in vivo mammalian counterpart, we examined mouse single cell data obtained from the posterior region of two E9.5 mouse embryos. Mouse and human count matrices were each normalized to 10,000 reads per cell, and only genes with greater than 0.02 mean and standard deviation across cells were used for further analysis. Mouse cells were clustered using Leiden clustering (resolution=0.03) and filtered for clusters that expressed SOX2+(neural marker) or TBXT+(NMP marker) but not TBX6 (mesodermal marker). After concatenating mouse dataset with our human dataset, which was pre-processed identically to before, we performed Louvain clustering (resolution=0.8) in the space of anterior and posterior transcription factors identified across clusters in human data alone in the space of bimodally expressed genes returned by the sparse multimodal decomposition (SMD) algorithm. The top differentially expressed genes between mouse and human species based on a Wilcoxon rank-sum test (HES5 and SOX3) were filtered out, and genes known to be differentially expressed between NMPs and non-NMPs were appended (SOX2 and TBXT). Human cells were subsampled to match the number of mouse cells (n=960 cells). Louvain clustering enabled classification of the combined human-mouse population into 5 cell types, including 3 neural tube progenitor cell types and 2 axial progenitor cell types. These corresponded to the 4 progenitor cell types identified from the in vitro human dataset, and NMPs from the in vivo mouse dataset. All NMPs in the combined dataset were derived entirely from mouse, while both mouse and human cells contributed to the other 4 cell types.

We evaluated the mean expression of all transcription factors in each cell type. Axial progenitors in the combined human-mouse dataset were all CDX2+HOXB9+, and were found to further cluster into two populations, consisting of TBXT+SOX2-low cells and TBXT-SOX2+ cells. We classified these populations as neuromesodermal progenitors (NMPs) and pre-neural tube (PNT) progenitors, respectively. All NMPs were derived from the mouse dataset alone, while the human dataset lacked NMPs. NMPs expressed several transcription factors in addition to high level of TBXT and a low level of SOX2, including LEF1, ZIC3, ETV4 and ETV5. PNT progenitors were transcriptionally distinct from NMPs, with a lack of TBXT expression and high levels of SOX2, GBX2, and ZIC5. Correlation analysis of human and mouse cell types revealed a high correlation of human progenitor cell types with mouse (r>0.7), whereas mouse NMPs poorly correlated with all human cell types (r<0.4). A Wilcoxon rank-sum test across NMP and PNT clusters revealed additional differentially expressed transcription factors between the two axial progenitor populations: ETS2, ARID3B, ARID1A, EVX1, HES7, SALL4, SALL3, and FOXD1 in NMPs and ZIC2, GBX2, GTF3A, HMGA1, ADNP, and SMARCA1 in PNT progenitors. As validation of these markers, several genes have been previously observed to mark the tailbud population in mouse embryos, including SALL4 and ETV transcription factors in NMPs and ZIC2 in PNT progenitors and play distinct roles in caudal morphogenesis. Other markers, such as ARID3B, have been shown to localize to the tailbud but as of yet have no known roles in this region of the embryo. Unlike axial progenitors, neural tube progenitors were all CDX2− and SOX1+MEIS2+. Within neural tube progenitors, spinal cord progenitors were enriched for posterior HOX genes including HOXB9 through HOXB3. Posterior hindbrain progenitors expressed higher levels of NR2F1, NR2F2, POU3F2, and PBX3, while anterior hindbrain progenitors expressed higher levels of IDI, MAFB, and anterior HOX gene HOXA2. A Wilcoxon rank-sum test pulled out these same sets of transcription factors between the two hindbrain populations, along with SOX4, HMGA1, and NR6A1 in posterior hindbrain and EGR2, HES5, IRX2, and POU3F1 in anterior hindbrain.

An unbiased analysis revealed differentially expressed genes between the two compartments. Several FGF and WNT ligands, including FGF8, FGF17, WNT5A, and WNT5B, were expressed in both populations, but at higher levels in NMPs than in PNT progenitors. WNT inhibitors SFRP1 and SFRP2 were also present in both cell types but expressed at higher levels in PNT progenitors relative to NMPs. Canonical WNT ligand WNT3A as well as canonical WNT inhibitors DKK1 and IGFBP4 were specifically expressed in mouse NMPs, while FGF ligand FGF2 was specifically expressed in human PNT progenitors and FGF ligand FGF3 was enriched in both NMPs and in hindbrain, but not PNT progenitors.

### In Situ Sequencing (STARmap)

Glass-bottom 24-well plates (Cellvis, P24-1.5H-N) were treated with oxygen plasma for 5 mins (Anatech Barrel Plasma System, 100 W, 40% 02) and followed by methacryloxypropyltrimethoxysilane (Bind-Silane) coating solution (88% ethanol, 10% acetic acid, 1% Bind-Silane, 1% H2O) treatment for 1 hour. The 24-well plates were rinsed with ethanol 3 times and left to dry at room temperature for 3 hours. Micro cover glasses (12 mm) were pretreated with Gel Slick at RT for 15 mins and air-dried before using.

Neural tube organoids were fixed with 1 mL 4% PFA for 30 mins at RT and then washed with PBS 3 times for 10 mins each time. The sample was permeabilized with 1 mL (0.1M glycine, 0.1 U/μL SUPERase. In, 0.5% Triton-X 100 in PBS) for 30 mins and then washed with 1 mL PBST (0.1% Triton-X 100 in PBS) 3 times for 10 mins each. The sample was then incubated in 1× hybridization buffer (2×SSC, 10% formamide, 1% Triton-X 100, 20 mM RVC, 0.2% SDS, 0.1 mg/mL yeast tRNA, and pooled SNAIL probes at 10 nM per oligo) in a 40° C. oven with shaking for 48 hours. The sample was washed with 1 mL PBSTV (1% RVC in PBST) at 37° C. 3 times for 20 mins each, and then washed with PBST three times for 10 mins each at RT The sample was then incubated in a 1 mL ligation mixture (1:50 T4 DNA ligase, 1:100 BSA, 0.2 U/μL SUPERase-In) at RT overnight and then washed with 1 mL PBST three times for 10 mins each. The sample was incubated in 1 mL RCA mixture (1:50 Phi29 DNA polymerase, 250 μM dNTP, 1:100 BSA, 0.2 U/μL SUPERase-In and 20 μM 5-(3-aminoallyl)-dUTP) at 4° C. for 1 hour before incubating at 30° C. for 6 hours and then washed with 1 mL PBST 3 times for 10 mins each. The sample was incubated with 20 mM methacrylic acid N-hydroxysuccinimide ester in PBST for 3 hours at RT and washed with PBST 3 times for 10 mins each. The sample was then incubated with monomer buffer (4% acrylamide, 0.2% bis-acrylamide, 2×SSC) overnight at RT The buffer was then aspirated, and 55 μL of polymerization mixture (0.2% ammonium persulfate, 0.2% tetramethylethylenediamine dissolved in monomer buffer) was added to the sample. The Gel Slick coated coverslip was immediately put on the sample, and the polymerization was conducted in an N2 container for 90 mins. The sample was then washed with PBST 3 times for 10 mins each.

Two cycles of sequencing experiments were performed to decode gene identity. Within each cycle, the sample was first treated with a stripping buffer (60% formamide, 0.1% Triton-X-100) at RT 3 times, 15 mins each, followed by PBST wash for 4 times, 10 mins each. Then the sample was incubated with the sequencing mixture (1:25 T4 DNA ligase, 1:100 BSA, 10 μM reading probe, and 5 μM fluorescent oligos) at RT for 12 hours. Then the sample was washed by the washing and imaging buffer (2×SSC, 10% formamide, and 0.1% Triton-X-100) 4 times, 10 mins each. DAPI was dissolved in PBST and used for nuclei staining for 2 hours before the first sequencing cycle. Finally, the sample was immersed in the washing and imaging buffer for imaging. Image acquisition was performed with Leica TCS SP8 confocal microscopy with 25× water-immersion objective (NA 0.95).

A data analysis pipeline was built with MATLAB 2019b to decode gene identity and quantify the gene expression level of each cell from the STARmap images. First, sequencing fluorescence images were preprocessed with top-hat filtering by a disk structuring element (radius=3) to remove the background noise. Second, the contrast of the image for each channel from the second sequencing cycle was adjusted to match the image from the first cycle with histogram matching function mismatching. Third, the composite fluorescence images for the second cycle were registered with the composite fluorescence image from the first cycle using a phase correlation algorithm followed by local distortion registration with function imregdemons. Fourth, the dots of amplicon locations were identified from images in the first cycle by a 3D regional maximum detection algorithm implemented in function imregionalmax. Then the dominant color of every identified dot in each cycle was determined by a 3×3×3 voxel volume surrounding its centroid location. The color sequence for each dot was decoded as a gene barcode and compared with the codebook. Fifth, cell segmentation was performed with ClusterMap and CellProfiler then RNA reads were assigned to the segmented cells, accordingly, described in detail as follows.

We first used the IdentifyPrimaryObjects function from CellProfiler to segment the DAPI stained nuclei images and then assign each decoded RNA to the segmented nuclei. We next applied ClusterMap to assign RNA out of the nuclei to the corresponding cell. ClusterMap identified cells by incorporating the physical location and gene identity and directly clustering spots of RNAs and DAPI signals. Specifically, to quantify and utilize gene expression information in each spot's local neighborhood, ClusterMap introduced a high-dimensional vector, termed neighborhood gene composition (NGC), which was computed by considering gene expression profiles in a circular window over each RNA spot. The NGC coordinates and physical coordinates of each RNA spot are then computationally integrated into joint physical and NGC (P-NGC) coordinates over each RNA spot. Next, ClusterMap aimed to cluster the RNAs in the P-NGC coordinates for downstream segmentation by density peak clustering (DPC). DPC identifies cluster centers with a higher density than the surrounding regions as well as a relatively large distance from points with higher densities. ClusterMap applied DPC to compute two variables: local density ρ and distance δ for each spot in the joint P-NGC space. For each spot, ρ value represents the density of its closely surrounded spots, and δ value represents the minimal distance to spots with higher ρ values. Spots with both high ρ and δ values are highly likely to be cluster centers. ClusterMap then ranked the product of these two variables, γ, in decreasing order to find genuine cluster centers with orders of magnitude higher γ values. After the cluster centers have been selected, the remaining spots are assigned to one of the clusters respectively in descending order of ρ value. Each spot is assigned to the same cluster as its nearest previously assigned neighbor, and each cluster of spots represents an individual cell. Outliers that were falsely assigned among cells can be filtered out using noise detection in DPC. ClusterMap showed identified cells with more precisely outlined cell boundary and illustrated cell morphology and consistent performance among various in situ transcriptomics datasets. The detailed introduction, discussion, and analysis of ClusterMap performance on cell segmentation can be found in our previous report.

R package Seurat3 was used for the single-cell gene expression analysis. Gene counts of each cell were normalized so that the total count of all genes in each cell equals 100. The normalized count value is then log-transformed with log(x+1). The data normalization was performed with the NormalizeData function in Seurat with a scale factor=100. Each gene in the cell-by-gene matrix was scaled to unit variance and zero mean with ScaleData function in Seurat followed by dimensionality reduction with principal components analysis (PCA) using RunPCA function in Seurat. Based on the explained variance ratio, the top 10 principal-components were used to construct the k nearest neighbor (kNN) graph for Louvain clustering using FindNeighbors and FindClusters function in Seurat. Uniform Manifold Approximation and Projection (UMAP) was used to visualize the 2D representation of each cell using the RunUMAP function in Seurat. Monocle was used to compute pseudotime along the cell trajectory with UMAP as input with the order_cells function in Monocle3. The CDX2 positive axial progenitor cells were specified as root cells for pseudotime prediction.

### Hybridization Chain Reaction (HCR)

HCR was performed on organoids following a previously published protocol. Briefly, after fixation with 4% PFA, organoids were washed twice in PBS+0.1% Tween20 (PBST) and dehydrated in increasing concentrations of methanol in PBST and stored at −20° C. Organoids were rehydrated in decreasing concentrations of methanol in PBST and treated with 25 μg/ml Proteinase K for 4 minutes at room temperature. After two washes in PBST, organoids were refixed in 4% PFA in PBST for 20 minutes at room temperature. After three washes in PBST, organoids were treated with 2 pmol hybridization probe in 0.5 ml probe hybridization buffer, and incubated overnight at 37° C. The following day, organoids were washed three times with probe wash buffer, and twice with 5×SSCT buffer. 6 pmol of amplifier hairpin h1 and h2 were snap-cooled by heating at 95° C. for 1.5 minutes and cooling to room temperature for 30 minutes, then added to organoids in 0.1 ml amplification buffer along with DAPI and incubated overnight at room temperature. The following day, organoids were washed twice in 5×SSCT and twice in PBST before transferring to a glass-bottom well plate for imaging. The following accessions were used to design hybridization probes: SOX2 (NM_003106), WNT5A (NM_003392), WNT5B (NM_032642).

### Cloning Guide RNA Plasmids

pgRNA-CKB and pLKO5.sgRNA.EFS.GFP vectors were digested with BsmB1 (NEB R0739) for 1 hour (hr) at 37° C. and gel purified. Guide RNA (gRNA) sequences were generated using the Broad Institute BPP sgRNA Designer where three gRNAs were designed per target gene. gRNA sequences were ordered as 20 bp single stranded oligos from Integrative DNA Technologies (IDT) containing the BsmBI-v2 (NEB R0739) restriction enzyme sticky end on the 5′ end of the forward oligo and the 3′ end of the reverse oligo. The two oligos per gRNA were duplexed together to generate double stranded DNA for cloning by mixing 25 μL of each 100 μM reconstituted oligo and 2 μL T4 Ligase Buffer (NEB B0202). The reaction was heated to 100° C. for 5 min and cooled to room temperature over 20 min. Duplexed gRNAs were diluted 1:10,000 in water and 14 μL added to a ligation reaction (NEB M0202). Ligation was allowed to proceed for 1 hr at RT, after which 2 μL was transformed into NEB Stable competent E. coli (NEB C3040H) competent cells by heat shock at 42° C. for 30 seconds. Colonies were PCR screened and verified by Sanger Sequencing via Genewiz Plasmids were grown and stored in NEB Stable E. coli. We generated at least 3 gRNA plasmids targeting each of CDX2, WNT5A, WNT5B, SFRP1, and SFRP2. Of these, a single gRNA was ultimately chosen for each gene based on the fluorescence intensity of the generated clones as a proxy for expression level in the cell.

The following guide RNA sequences were used for line generation: mKate2 control (GGAGACGTGACCGTCTCT) (SEQ ID NO: 1), GFP control (GGAGACGGACGTCTCC) (SEQ ID NO: 2), sgCDX2 (TCTGCAGCCTAGTGGGAAGG) (SEQ ID NO: 3), sgWNT5A (CCTGAGTGAATTACCCAGGA) (SEQ ID NO: 4), sgWNT5B (GGGGAAGTTTCGGCCCAAGT) (SEQ ID NO: 5), sgTBXT (TATTCCACTTGAACTCCCCA) (SEQ ID NO: 6), sgSFRP1 (CGGAGGTGCGGCGAGCAGGA) (SEQ ID NO: 7), and sgSFRP2 (ACGGCTCATTCTGCTCCCCC). For verification of integration of the gRNAs by Sanger sequence, we used oligos 5′ GAGTTAGGCAGGGATATTCACC 3′ (SEQ ID NO: 8) for the pgRNA-CKB vector and 5′ CTGCCCCGGTTAATTTGCA 3′ (SEQ ID NO: 9) for the pLKO5.sgRNA.EFS.GFP vector. pgRNA-CKB was a gift from Bruce Conklin (Addgene #73501). pLKO5.sgRNA.EFS.GFP was a gift from Benjamin Ebert (Addgene #57822).

### Lentiviral Transduction of Guide RNAs

One day before viral production, Lenti-X 293 T HEK cells (Takara Bio 632180) were split 1:10 into a tissue culture treated 6-well plate (Stellar Scientific CT229106) in 10% fetal bovine serum (FBS) in DMEM/F12 (Thermo Fisher 11039021) and left to grow overnight at 37° C. and 5% CO₂. The next day, plasmids for second generation lentiviral production: envelope expressing plasmid pMD2.G+ packaging plasmid psPAX2+ guide RNA plasmid were mixed in 0.197:0.355:0.449 molar ratios, respectively, in 200 μL JetPRIME Buffer+4 μL JetPRIME Reagent (Polyplus, VWR 114-07). The reaction was incubated for 10 min at room temperature and added dropwise to HEK cells for transfection. 4 hrs post transfection, media was removed from HEK cells using lentiviral BSL2+ safety protocols and replaced with 5% FBS in DMEM/F12. Media containing virus was harvested 24- and 48-hours post-transfection and then concentrated using Lenti-X Concentrator (ClonTech 6311231). First, media was spun at 500 g for 15 min and supernatant was transferred to a new conical tube. 2 mL of Lenti-X Concentrator was added and mixed by gently flicking. The mixture was incubated for at least 30 min incubation at 4° C. and spun down at 1500 g for 45 min at 4° C. to pellet virus. Supernatant was disposed of in bleach and viral pellet was resuspended in 200 μL mTeSR Plus and stored at 4° C. for up to 2 weeks. pMD2.G and psPAX2 were gifts from Didier Trono (Addgene #12259, Addgene #12260).

Lentiviral titrations were pre-determined using a serial dilution assay on stem cells. Once a titer was determined, the volume of virus was added dropwise to hPSCs. Cells were treated as lentivirus positive and handled in BSL2+ conditions for at least 3 days post transduction. Media was changed daily with mTeSR Plus and cells were washed 3 times with DPBS (Lonza). After the third day cells were removed from secondary containment and treated as lentivirus negative samples. Cells were sorted by FACS (based on either mKate2 or GFP fluorescence) six days post transduction. To transduce micropatterns directly, harvested virus was added dropwise to micropatterned cells several hours after seeding. To simultaneously knock down SFRP1 and SFRP2, HEK cells were separately transfected by an mKate2-expressing SFRP1 gRNA construct and a GFP-expressing SFRP2 gRNA construct. Harvested viruses were pooled and added to hPSCs, and FACS was performed for mKate2+GFP+ cells.

### Fluorescence-Activated Cell Sorting for Cell Line Generation

Accutase-dissociated cells were sorted using a BD Aria III (BD Biosciences) using a 100 μm nozzle. Cells were gated such that the RFP+ or GFP+ population was taken as the cells with the top 1% fluorescence intensity in FITC or PE channels. One cell was sorted per well of a 96 well plate filled with 100 μL of expansion media, composed of mTeSR Plus supplemented with 10 μM Y-27632 and 1×Clone® (Stem Cell Technologies 5889). Expansion media was replaced every 2 days until cells formed confluent colonies, then passaged into a 6-well plate where they were maintained as normal cell lines. In all cases, cell lines with the brightest mKate2 or GFP fluorescence were selected for mutant analysis. Lines were subsequently screened for a phenotype in our organoid differentiation assay.

### Mathematical Model of Symmetry Breaking

Here we provide the reader with a mathematical intuition for why coupling between organoids coordinates their symmetry breaking. As stated in the main text, physical systems with coupled degrees of freedom have a low entropy ground state with broken symmetries. The question is whether we can similarly couple organoids to make their symmetry breaking effectively deterministic. The cells of the organoid are not just passive recipients of signals. In response, they secrete signals of their own. This is the fact that we take advantage of two couple organoids.

To appreciate qualitatively how this might happen, consider a simple model where a symmetry breaking event occurs, leading to appearance of two distinct cell types, anterior and posterior. Cells respond to an activator W by adopting the posterior fate. Additionally, cells secrete a diffusible signal molecule S that opposes the activator by inhibiting the posterior fate and promoting the anterior fate.

Let the signal S have a lifetime r and a diffusion constant D. Thus, the concentration of S, denoted by S for simplicity, obeys the following equation: ∂S∂t=D∇2S−Sτ. The fate adopted by the cells is θ(W−S) where θ(x) is the Heaviside step function, and 1 represents a posterior fate, while 0 represents an anterior fate. Therefore, when S>W as measured by a cell, it adopts an anterior fate, while when S<W it adopts a posterior fate. This is equivalent to an infinite Hill coefficient term driving the expression of the anterior and posterior targets.

We can intuitively guess what will happen: molecules of S will diffuse a distance Dτ before they are absorbed or otherwise disintegrate. If any region of neighboring organoid is roughly within this distance, the S secreted by the first organoid will combine with the S secreted by the second organoid. Otherwise, the first organoid will have no effect on the second. Therefore, if the organoids are arranged in the right way, it will be possible to get them all to coherently make S in the regions where they are close to each other, robustly inhibiting the posteriorizing effect of the activator Win those regions.

To go beyond these intuitive arguments, we implemented a simple simulation in MATLAB, consisting of a 2D representation of our experiment. We used the built-in partial differential equation solver in order to evolve the concentration of S in time. Using parameter values D=0.25 and r=1, we simulated 6 organoids arranged hexagonally in a similar fashion to our experimental setup, with an edge-to-edge spacing of 0.5 arbitrary length units. The circular organoids are defined as sources of the signal and thus have a Neumann boundary condition with a constant production rate ∂S∂t=a, with set to 1, while the edges of our system are held at S=0 for all time t. To initialize the simulation, we start with a value of S=0 everywhere in space. We then evolved the simulation in time steps of δt=0.1 for 250 steps, by which time the profile of S had reached a steady state.

The model above considered a secreted signal that diffuses. The increased concentration of the signal in the interior is solely due to the proximity of the organoids. If in addition, there is positive feedback (in response to S, the organoids secrete more S), this effect will be further enhanced.

### Mathematical Model of Excitable System

Here we provide a mathematical intuition for how the excitable system underlying axial elongation is prone to instabilities. As stated in the main text, posterior signals such as FGF and WNT are produced in the posterior region of organoids. If these signals are involved in a positive feedback loop, then they have the capacity to trigger runaway signaling events. These runaway events can be prevented by secreted inhibitors of FGF and WNT signaling.

To appreciate how this may happen, we build upon our mathematical model of a generalized diffusible inhibitor. We again assume that the inhibitor is secreted uniformly across the organoid with lifetime r and diffusion constant D, i.e., ∂S∂t=D∇2S−Sτ. In addition, we assume that activator W activates more W secretion, and enhances W signaling activity. We also assume that W is much less diffusive than its inhibitors and set its diffusion constant to zero. We assume that S inhibits W by binding to it, making it incapable of binding to the cell surface receptor (as is the case with many secreted inhibitors). Thus, the effective level of W at any point in space is W_{eff}=W−S at that location. We then write the dynamics of activator production at each location r→ on the surface of the organoid to be ∂W∂t=B(WeffW0)n1+(WeffW0)n−Wτ where S is the concentration of the inhibitor at that location, τ is set to 1 representing the degradation of the molecule over time, and the second term represents a nonlinear feedback of the activator onto itself. The Hill coefficient n is set to 3. Starting the system with the initial activator concentration throughout the organoid at W(t=0)=0.5, we chose parameter values B=1, S₀=0.3, W₀=0.2, such that Wₘₐₓ=1 and ∂W∂t=0atS=S0. We evolved the values of W with S already in its steady state profile. We used time steps of δt=0.001 for 10,000 steps, by which time the profile of W had reached a steady state, and a clear elongation front was apparent. In addition, we perturbed the steady state with an ectopic pulse of activator (W=0.5) far away from the tailbud in two conditions. First, levels of S were determined by the steady state concentration of S according to its partial differential equation. Secondly, levels of S were turned to 0 everywhere. This pulse was also allowed to evolve for 10,000 steps of δt=0.001.

### Quantification and Statistical Analysis

### Gene Expression and Length Quantification Along Organoid A-P Axis

To quantify gene expression along the A-P axis of each organoid, gene expression images were projected onto the organoid midline, computed by skeletonizing the segmented image using the skimage package in Python. For all experiments, a gene expression domain was counted as present in an organoid if it contained a region that exceeded the half-maximal expression of the gene under control conditions (except in the case of TBXT, where it was defined as the half-maximal expression under FGF pulse conditions).

To quantify A-P axis lengths, segmented regions of interest were used to compute either the Feret diameter or major axis corresponding to the ellipse of best fit using the minEnclosingCircle and fitEllipse functions from the cv2 package in Python. The Feret diameter was used for all length quantification unless otherwise specified but was highly correlated with major axis across timepoints (r=0.99). For experiments where time series information was available, the relative A-P length on day 4 was computed by dividing by the length on day 1.

Proliferation Index Quantification Tailbud proliferation index was defined as the ratio of pHH3+ cells to DAPI+ cells in either the CDX2+ or SOX1− region of the organoid, depending on the antibody stain used. Due to the difficulty of segmenting cells in the DAPI channel, the DAPI+ area of occupied by the organoid was divided by the average area of a pHH3+ cell across organoids to determine the effective number of cells. This likely produced an underestimate of the number of cells, and therefore an overestimate of the fraction of cells undergoing mitosis, as DAPI+ nuclei were pseudostratified and therefore multi-layered, while pHH3+ nuclei were apical only. Thus, we dubbed this measure as the tailbud proliferation index, which enables comparisons of relative proliferation rates between organoids.

### Example 3. Controlling Human Organoid Symmetry Breaking Reveals Signaling Gradients Drive Segmentation Clock Waves

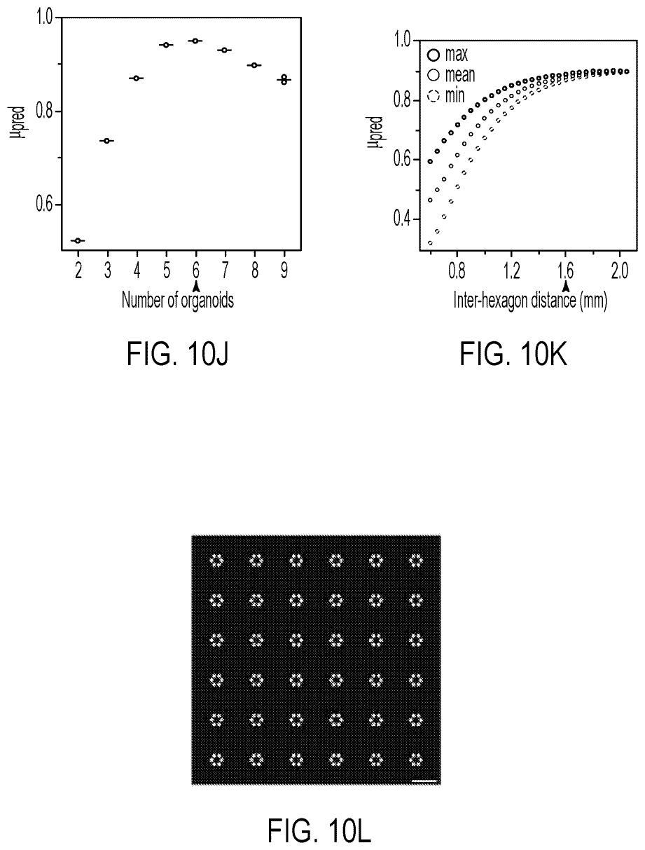

Axial development of mammals involves coordinated morphogenetic events, including axial elongation, somitogenesis, and neural tube formation. To gain insight into the signals controlling the dynamics of human axial morphogenesis, we generated axially elongating organoids by inducing anteroposterior symmetry breaking of spatially coupled epithelial cysts derived from human pluripotent stem cells using the methods described in Example 1. Each organoid was composed of a neural tube flanked by presomatic mesoderm sequentially segmented into somites. Periodic activation of the somite differentiation gene MESP2 coincided in space and time with anteriorly traveling segmentation clock waves in the presomatic mesoderm of the organoids, recapitulating critical aspects of somitogenesis. Timed perturbations demonstrated that FGF and WNT signaling play distinct roles in axial elongation and somitogenesis, and that FGF signaling gradients drive segmentation clock waves. By generating and perturbing organoids that robustly recapitulate the architecture of multiple axial tissues in human embryos, this work offers a means to dissect mechanisms underlying human embryogenesis. See FIG. 11. A complete description of these studies can be found in Yaman Y I and Ramanathan S. Cell. 2023 Feb. 2; 186(3): 513-527, the content of which (including supplemental materials) is incorporated herein in its entirety (Yaman 2023).

Here, we aimed to characterize the dynamics of human axial patterning and morphogenesis to understand how segmentation clock waves interact with signaling gradients. Following the methods developed in Examples 1 and 2, we employed a combination of machine learning and bioengineering tools to tune the coupling of human pluripotent stem cell organoids by controlling their spatial arrangement. This enabled us to generate hundreds of in vitro organoids simultaneously, each robustly and reproducibly recapitulates the architecture of axial tissues in human embryos. Using single-cell sequencing and computational analysis, we validated the organoids by determining the cell type composition and the spatial profiles of key transcription factors and signaling molecules along the anteroposterior axis. We demonstrated that the organoids recapitulate the dynamics of axial elongation, anteriorly moving segmentation clock waves, posteriorly moving somite determination front, and somite segmentation. By perturbing the organoids, we showed that FGF signaling gradients drive the anterior propagation of segmentation clock waves while simultaneously controlling the movement of the somite determination front, somite segmentation, and, together with WNT, axial elongation. We finally discuss the implications of these results in the context of the existing models for axial patterning of the somites.

### Spatially Coupled Organoids Achieve Robust A-P Symmetry Breaking

To understand human axial development, we aimed to generate hundreds of organoids, each with an axially extending tailbud that generates a single lumen neural tube flanked posteriorly by PSM and anteriorly by somites as seen in vivo. To achieve this desired outcome, we built upon a bioengineering and machine learning framework developed in a companion manuscript, to reproducibly break anterior-posterior (A-P) symmetry. We first recapitulated the human epiblast by micropatterning human pluripotent stem cells at random locations on a glass coverslip and folding them into 150 μm diameter cysts composed of a single epithelial layer of pluripotent stem cells enclosing a lumen²⁵. To induce differentiation, we exposed the cysts to medium containing WNT agonist CHIR99021 (at concentrations ranging from 2.5 μM to 6 μM) while inhibiting BMP (LDN193189, 0.5 μM) and TGF-β signaling (A83-01, 0.5 μM). After 48 hours, we stained the differentiated organoids for SOX2 and TBXT. These markers were chosen to label the SOX2+TBXT+ neuromesodermal (NMP) progenitors in the tailbud, SOX2−TBXT+ paraxial mesoderm flanking the neural tube posteriorly, and the SOX2+TBXT− cells of the neural tube. In the desired organoid morphology, we expected TBXT to be posteriorly expressed relative to SOX2. We thus scored our organoids using a polarization metric, μTBXTSOX2 measured, defined as the distance between the centroid of TBXT+ cells and that of SOX2+ cells in each organoid. The organoids on the random pattern showed a large variability in μTBXTSOX2 measured. Using the approach in Anand et al., we optimized the spatial arrangement of the differentiating organoids on a coverslip and the CHIR concentration such that each organoid broke A-P symmetry to acquire a large μTBXTSOX2 measured. This arrangement consisted of 150 μm diameter organoids in groups of four at the vertices of 200 by 200 μm squares. After exposure to 4 μM CHIR for 48 hours, every organoid broke A-P symmetry and was polarized with low SOX2/high TBXT posteriorly and high SOX2/low TBXT anteriorly. To visualize this polarization in live organoids, we used a dual fluorescence reporter HES7:Achilles/MESP2:mCherry iPSC line, allowing us to monitor the polarization of the presomatic mesodermal marker HES7. See also Yaman 2023.

Polarized Organoids with Posteriorly Localized NMPs Generate a Single-Lumen Neural Tube Flanked by Segmented Somites

After the initial A-P symmetry breaking at 48 hours, randomly selected 96 organoids of the 144 on the same coverslip were removed and cultured individually in low adhesion 96 well plates in basal media (E6) supplemented with Matrigel, without any signaling molecules or inhibitors. From 72 to 120 hours after the start of differentiation, all the organoids that were successfully transferred to the 96 well plate underwent axial elongation. They also contained a TBXT+SOX2+ NMP population at the posterior tip that was maintained throughout 120 hours of differentiation. Every organoid showed lateral expression of the somitogenesis marker MESP2 based on mCherry expression and displayed anterior to posterior progression of the MESP2+ somite fate (n=95).

We next determined the expression patterns of key proteins in these organoids at 120 h through immunostaining. Every organoid had an anteriorly positioned SOX1+ and SOX2+ neural tube with a single lumen and the proper apicobasal polarity as shown by ZO-1 and N-cadherin stains marking the apical tight junctions between epithelial cells. In each organoid, the neural tube was flanked posteriorly by TBX6+ paraxial mesoderm and anteriorly by the MESP2+ somite cells. ZO-1 and N-cadherin expression was localized in multiple foci in the MESP2+ somite region, showing the segmented architecture of somites flanking the anterior neural tube. Similar to the A-P organization of the developing embryo, the TBX6-expressing domain formed a clear boundary corresponding to the somite determination front, posterior to the MESP2+ cells. By 120 hours of differentiation, the MESP2 expression pattern had resolved into an alternating pattern in each segment as seen in vivo, indicating that the somites displayed A-P compartmentalization. These results together demonstrate that our approach robustly achieved the differentiation of pluripotent stem cells into organoids with correctly positioned tailbud, neural tube, and somite structures, capturing key aspects of in vivo axial development. See also Yaman 2023.

### Clustering and Diffusion Map Analysis of Axial Organoid Transcriptome Reveals Cell Types and A-P Organization of Neural Tube and Paraxial Mesoderm

To explore the cell type composition of organoids, we performed single-cell RNA sequencing of 11009 cells obtained from 10 organoids at 120 h. Clustering and identifying cell types from this data requires measuring the distance between cells in gene expression space. The Euclidean distance in the space of all high-variance genes leads to incorrect clustering and classification. To overcome this challenge, we previously developed and validated an unsupervised statistical method, sparse multimodal decomposition (SMD), to identify the key subset of genes that can be used to determine cell types. Using SMD, we identified 48 key genes with significant z-scores from the single-cell data. Through hierarchical clustering in this gene subspace, we identified seven cell types in the organoids. The first cluster co-expressed TBXT and SOX2, indicating a neuromesodermal progenitor identity-. The second cluster, consisting of SOX2+TBXT− cells, co-expressed tailbud genes HOXA10, CDX2, and NKX1-2, consistent with a pre-neural tube identity. A third cluster possessed a neural progenitor identity, expressing neural markers SOX1, PAX6, HES5, and IRX3 along with high levels of SOX2. Three additional clusters were associated with paraxial mesodermal identity. These included a presomatic mesoderm-like cell cluster expressing TBX6, MSGN1, and HES7, an early somite cell cluster expressing MEOX1, TCF15, and RIPPLY1, and a mature somite cell cluster expressing PAX3, TWIST1, and FST. Lastly, we identified a small number of notochord cells expressing SHH, NOTO, and high levels of TBXT.

To map the spatial distribution of neural and mesodermal lineages present in organoids from scRNA-seq data, we constructed a diffusion map in the subspace of genes identified by SMD. We previously showed that such a map could recapitulate the spatial ordering of cells along the A-P axis, allowing the inference of spatial profiles of gene expression. We plotted the expression levels of genes arranged in order of the position of their peak expression level along the inferred posterior to anterior axis in both the neural and mesodermal tissues. In the mesodermal cells, genes expressed in the tailbud progenitors (TBXT, CDX1, CDX2, CDX4, LIN28A, HOXA10, HOXC10) peak most posteriorly, followed by the marker genes of presomatic mesoderm (TBX6, DLL3, DLL1, MSGN1), followed by the somite determination front marker MESP2, then early somite markers (MEOX1, RIPPLY1, TCF15), and finally mature somite markers (SIX1, PAX3, ALCAM). Along the neural lineage, cells closest to the neuromesodermal progenitors expressed pre-neural tube markers CDX2, MSX1, and NKX1-2. On the other hand, anteriorly positioned cells expressed higher levels of neural progenitor markers SOX1, PAX6, OLIG3, and IRX2, together with higher levels of SOX2. We verified the inferred anterior-posterior (A-P) expression profiles of the marker genes by MESP2:mCherry reporter expression and immuno-staining against TBXT, TBX6 and for mesodermal cells and SOX2, SOX1 and PAX6 for neural cells. See also Yaman 2023.

### Inferring the Anteroposterior Profiles of WNT, FGF, RA, NOTCH, and BMP Signaling Pathway Components in the Mesodermal and Neural Tissues

We next tested whether our organoids recapitulated the anteroposterior expression gradients of WNT, FGF, RA, and NOTCH signals and their targets as observed in vivo in mouse, chick, and zebrafish embryos. The expression profile of the detected FGF ligands in neural cells (FGF8, FGF17) and mesodermal cells (FGF3, FGF4, FGF8, FGF17) were localized most posteriorly in each lineage. FGF receptors were differentially expressed between the mesoderm and neural tissues, with FGFR1 expressed throughout the mesoderm and FGFR2 showing monotonically increasing levels from posterior to anterior in the neural tube. In both tissues, FGF target genes SPRY4 and ETV4 were upregulated posteriorly, consistent with the role of FGF in maintaining the axial progenitor state in the tailbud of the mouse embryo.

Like FGF, all detected WNT ligands showed a posterior to anterior graded expression. While both canonical (WNT3A, WNT8A) and noncanonical (WNT5A, WNT5B) WNT ligands were expressed in the mesodermal tissue, only noncanonical (WNT5A, WNT5B) WNT ligands showed expression in the neural tissue. The WNT ligand expression gradient was opposed by an expression gradient of secreted WNT inhibitors SFRP1 and SFRP2 in both mesoderm and neural lineages. WNT targets (CDX2, CDX1, SP5) showed a posteriorly restricted expression pattern in the neural tissue similar to that of WNT ligands. In the mesodermal tissue, one class of WNT targets (TBXT, CDX2, CDX4), was highly expressed at the posterior end of the tissue and downregulated anteriorly. The second class of targets (AXIN2, DLL1) and the WNT transcriptional mediator LEF1 showed peak expression at the anterior end of the presomatic mesoderm closest to the somite determination front.

Retinoic acid signaling is important in mouse embryos for the fate specification of NMPs, differentiation of presomatic mesoderm, and the patterning of the neural tube. We observed that in our organoids, while the retinoic acid receptor gamma (RARG) was expressed throughout the neural and mesodermal tissues, the retinoic acid synthesis gene ALDH1A2 was expressed only in the somites anteriorly. Anterior retinoic acid secretion from somites combined with the posterior expression of the retinoic acid degradation enzyme CYP26A1 is consistent with an A-P retinoic acid gradient. Anterior upregulation of transcription factor PAX6 known to be downstream of retinoic acid signaling suggested that the neural tube was patterned by retinoic acid secreted by the flanking somites. Consistent with this, immunostaining showed upregulation of PAX6 protein in the section of the neural tube in proximity to the somites.

Next, we investigated the NOTCH signaling pathway, a key component in regulating periodic somite segmentation and neurogenesis. In the mesodermal lineage, both NOTCH pathway ligands (DLL1, DLL3), receptor (NOTCH1), and targets (HES6, HES7) were highly expressed in the presomatic mesoderm and downregulated anterior to the somite determination front. Although the NOTCH receptor expression levels were very low in the neural tissue, NOTCH ligand DLL1 and NOTCH target gene HES5 were upregulated in the neural progenitors anteriorly, suggesting the initiation of neurogenesis.

To validate the computationally inferred anteroposterior expression profiles of genes involved in signaling, we performed in situ hybridization chain reaction (HCR) for WNT3A, FGF8, FGF4, CYP26A1, and ALDH1A2 on organoids upon 120 h of differentiation. Consistent with the inferred profiles, WNT3A (n=3), FGF8 (n=5), FGF4 (n=3), and CYP26A1 (n=3) had posterior to anterior graded expression, while ALDH1A2 (n=3) expression was localized anteriorly at somites. Further, to determine whether the anteroposterior expression gradients of WNT and FGF ligands were reflected in the signaling pathway activity, we immunostained organoids for dephosphorylated ERK (dpERK) and β-catenin. Nuclear segmentation and quantification of dpERK and β-catenin signals showed that the signaling activity for both FGF and WNT pathways was high at the posterior tip and gradually decreased anteriorly. In total, our human organoid model shows anteroposterior expression gradients of WNT, FGF, RA, and NOTCH signals and targets consistent with in vivo observations in mouse, chick, and zebrafish embryos.

The dorsoventral patterning of somites and the neural tube is regulated by opposing gradients of BMPs secreted by surface ectoderm, roof plate, and somites, and SHH secreted by the notochord and floor plate⁴³. While our organoids lack roof plate and floor plate cell types, BMP7 was expressed by neuromesodermal progenitors and the posterior presomatic mesoderm, and BMP4 and BMP3 were expressed by somites. The presence of dorsalizing signals was consistent with acquiring a dorsal identity by both neural progenitor cells and somite cells. Neural progenitor cells expressed dorsal neural tube markers MSX1, OLIG3, IRX3 and PAX3, and somite cells expressed a dermomyotome marker PAX3, seen only dorsally in somites in vivo. In contrast, the ventral somite marker PAX1 was not expressed in any cells, consistent with the lack of SHH expression by cells. We also observed the expression of a neural crest marker SOX9 and the epithelial-to-mesenchymal transition gene SNAI2 in a subset of neural progenitors. Thus, in the absence of SHH, tailbud progenitors generated only dorsal neural and mesodermal cell types, possibly through the dorsalizing effect of BMPs secreted by mesodermal cells.

We compared our single cell RNA sequencing data with a previously published single cell RNA sequencing data obtained from E9.5 mouse embryo tail bud. The cell types identified in our axial organoid model were consistent with their in vivo counterparts. See also Yaman 2023.

Axial Organoids Show Sequential Somite Segmentation Coordinated with Traveling Segmentation Clock Waves

Given that the morphology, composition, and signaling profiles of 120-hour-old organoids were consistent with those of mammalian embryos, we next measured the dynamics of somitogenesis. We tested whether the organoids showed anteriorly propagating segmentation clock waves in the PSM and a coordinated posteriorly propagating somite determination front. We performed time-lapse imaging of organoids built with the dual reporter HES7-Achilles/MESP2-mCherry iPSC line. After 72 h of differentiation, when the first somite cells appeared at the anterior end, we transferred organoids to individual wells of a glass-bottom 96-well plate for imaging. Each organoid showed oscillating HES7 expression and posteriorly propagating MESP2 expression. Quantification of Achilles and mCherry signals showed that the MESP2+ region expanded in a step-like fashion. Each step in the MESP2 profile coincided with a peak of HES7 oscillations at the anterior end of the PSM, indicating that the timing of somite differentiation is coordinated with the segmentation clock wave in the axial organoids. Characterizing the HES7 oscillations along the A-P axis showed transient dynamics with almost synchronous oscillations throughout the PSM when the first somites appear around 72 hours. This is consistent with recent reports in mouse. By 96 hours of differentiation, a global phase gradient was established in 91% of the organoids (n=54). In these organoids, oscillations at the anterior tip lagged the posterior tip, resulting in anteriorly moving traveling waves. By 115 hours of differentiation, all organoids showed anteriorly propagating traveling waves with π/2 radians phase difference between posterior and anterior PSM on average (n=14). We also found that the oscillations were faster posteriorly compared to the anterior, with a period of 4.5 hours at the posterior tip and around 5 hours at the anterior PSM. In mouse, chick, and zebrafish embryos, the NOTCH pathway has been implicated in driving intracellular gene expression oscillations and somite segmentation. In line with in vivo studies, inhibition of NOTCH signaling through DAPT (25 μM) treatment resulted in the downregulation of HES7 oscillation amplitude and the impairment of MESP2 progression consistent with MESP2 being a NOTCH target gene. Thus, our in vitro organoid model recapitulates traveling segmentation clock waves and sequential somite segmentation as observed in vivo. See also Yaman 2023.

FGF and WNT Pathways have Complementary Roles in Axial Elongation, Movement of the Somite Determination Front, and Somite Segmentation.

In our organoids, WNT inhibition using the WNT secretion inhibitor IWP-2 (2 μM) added during somitogenesis resulted in truncated organoids with truncated PSM. However, we did not observe an effect of WNT inhibition on the movement of the somite determination front. We continued to see step-like segmentation with segment sizes similar to control organoids. Consistently, when ectopic WNT activation was uniformly stimulated by CHIR (3 μM) addition at 96 h of differentiation, organoids were elongated and developed a longer PSM compared to controls. These organoids continued to show step like MESP2 progression without a significant change in somatic mesoderm length or defects in segmentation compared to control organoids. These results show that the WNT pathway directly promotes axial elongation and has little effect on the progression of the determination front or somite segmentation.

Conversely, experiments in mouse have indicated that WNT plays a role in defining the position of the determination front. These results were obtained in mice through β-catenin deletion or stabilization. Unlike experiments in organoids or zebrafish, where the perturbation can be carefully timed, the effects of these mutations last throughout development, making it difficult to distinguish direct from indirect effects. Indeed, levels of FGF ligand and signaling activity were also significantly affected in these mutants, making it difficult to disentangle the effects of WNT perturbations from the downstream effects through FGF. Therefore, we next tested the role of FGF signaling in axial morphogenesis.

Consistent with studies in chick and zebrafish embryos, FGF inhibition in the organoids using FGF/ERK inhibitor PD0325901 (1 μM) led to truncation, with both the total organoid length and PSM length being shorter than control. FGF inhibition further led to an accelerating somite determination front propagation, and somite segments were about twice as large as control organoids. To observe the effects of FGF activation during somitogenesis, we exposed organoids to FGF4 (100 ng/mL) ligand at 96 h of differentiation. Uniform FGF4 treatment transiently decreased the size of the PSM. The truncation of the organoid compared to control is eventually due to the shortening of the somatic mesoderm. This suggests that a gradient of FGF activity is required for elongation, consistent with the literature. FGF4-treated organoids had a decelerated determination front progression and disrupted somite segmentation, as seen by the lack of ZO-1 foci or segments in the perturbed organoids. By performing β-catenin and dpERK immunostaining on the organoids treated with CHIR and FGF4, we validated that there was no cross-activation between WNT and FGF pathways after exposure for one oscillation period (4.5 hours).

These results indicate that while both FGF and WNT pathways are important for the axial elongation of both the neural tube and the mesoderm, the FGF pathway additionally plays a direct role in the definition of the somite determination front and the segmentation of the somites. See also Yaman 2023.

### FGF4 Gradients are Required for the Propagation of Segmentation Clock Waves

How waves of gene expression travel anteriorly along the presomatic mesoderm remains unknown. Furthermore, we do not know how the waves interact with the FGF and WNT signaling gradients. To determine if diffusible signaling gradients played a role in the propagation of segmentation clock waves, we wanted to place isolated colonies of PSM cells at a distance from each other to determine whether they could still communicate and mutually coordinate their segmentation clock oscillations through diffusive signals. Our logic was that if, in any configuration of these colonies that were not physically in contact, we could see coherent wave propagation across the colonies, it would suggest a role for diffusive molecules in the spatial coordination of the segmentation clock. We, therefore, performed the experiments for different spatial configurations of colonies, from those with uniform spacing between the colonies to those with a gradient of colony density. To achieve this, we micropatterned pluripotent stem cell colonies in proximity to each other on a coverslip and constrained colony expansion and cell migration by passivating the coverslip surface. The colonies could not touch each other, preventing communication between colonies through juxtacrine signaling (such as through NOTCH or YAP). By treating cells with CHIR and LDN for 48 hours, we differentiated colonies to a presomatic mesoderm identity. At the end of 48 hours, we replaced differentiation media with basal media and recorded HES7 oscillations. If diffusible signals did not play a role in generating traveling waves, we expected not to see a phase gradient across the colonies and, concomitantly, no propagation of NOTCH activity waves sweeping coherently across the colonies. In uniformly spaced colonies, time-lapse imaging revealed that the HES7 oscillations were synchronous. Contrarily, HES7 oscillations were spatially coordinated between the microprinted colonies in the patterns that had a radial density gradient (inter-colony distance increases with the distance from the center), and a phase gradient of HES7 oscillations was established, resulting in HES7 waves propagating from colonies at the edge to those in the center of the pattern. These results show that when colonies that are not in contact are arranged appropriately in space, their oscillation dynamics are coupled, leading to waves that travel across the colonies. Thus, diffusible signals could be important for the propagation of segmentation clock waves.

We, therefore, investigated whether FGF and WNT signaling could modulate the propagation of the measured HES7 waves in organoids. We observed that FGF inhibition resulted in the downregulation of the amplitude of HES7 oscillations. In contrast, WNT inhibition did not affect the oscillations, indicating that the FGF pathway is not only involved in defining the somite determination front and regulating somite segmentation but also in regulating the segmentation clock. We next uniformly activated FGF and WNT pathways in organoids. Time-lapse imaging of organoids revealed that uniformly activating the WNT pathway by adding CHIR did not affect the phase gradient along the A-P axis compared to the control organoids, showing that the WNT pathway has no direct effects on the dynamics of the segmentation clock. Treating organoids with FGF4 ligand resulted in synchronization of the oscillations throughout the A-P axis and loss of the traveling waves. In contrast, in control organoids, the segmentation clock waves propagated anteriorly through the PSM. Further, FGF4 addition accelerated the oscillations anteriorly, resulting in anterior and posterior PSM to oscillate with the same period. Similarly, adding FGF4 to microprinted colonies accelerated oscillations and abolished the traveling waves. Thus, we concluded that the FGF signaling gradient drives the propagation of segmentation clock waves during human somitogenesis through frequency modulation.

In physical systems that break symmetry, coupling the underlying degrees of freedom can substantially reduce the entropy of the broken symmetry state. Demonstrating the power of the approach developed in Anand et al., our study shows that by similarly coupling organoids, one can reduce the entropy of the broken symmetry state to obtain robust differentiation. The resulting axial organoids recapitulate critical aspects of axial development, including axial elongation, single-lumen neural tube formation, traveling segmentation clock waves, and sequential somite segmentation. These organoids allow us to image and deliver perturbations with temporal precision and at timescales corresponding to the clock oscillation times to extract mechanistic insight. Such an ability will enable us to study mammalian, specifically human axial development and associated diseases.

The accessibility of the organoids allowed live imaging, which is challenging in vivo in mouse, and impossible in human. Interpreting the effects of in vivo genetic perturbations is also challenging as the readouts of these perturbations are often indirect. In contrast, timed perturbations and simultaneous measurements of signaling pathway activity are easily achieved in organoid systems. In conjunction with the reproducibility of our organoids, such an ability is essential for achieving the reported results. Together, the experiments enabled us to obtain three significant insights about axial patterning and morphogenesis. First, we showed that WNT and FGF pathways together drive axial elongation. The absence of either of these signaling pathways leads to axis truncation. Moreover, unlike the WNT pathway, uniform activation of the FGF pathway also leads to axis truncation, highlighting the requirement of an FGF gradient for axial elongation. Second, by observing the immediate changes in determination front position upon temporally controlled inhibition of the FGF and WNT pathways, we could disentangle the roles of these two pathways in axial extension and somite patterning. We showed that the position of the determination front is determined solely by FGF and not by WNT signal. Third, we showed that the FGF signaling gradient along the A-P axis drives segmentation clock waves by modulating the period of the NOTCH oscillations at the cellular level. Posterior to anterior FGF activity gradient thus generates a frequency and phase gradient along this axis, leading to anteriorly traveling segmentation clock waves. Consequently, uniform activation of the FGF pathway results in synchronous oscillations throughout the A-P axis and a loss of traveling segmentation clock waves by accelerating the oscillations anteriorly. A simple mathematical model in which the level of FGF pathway activity determines both the oscillation frequency of the NOTCH targets and the determination front position is sufficient to produce anteriorly traveling clock waves, posteriorly moving determination front, and anteroposteriorly polarized somite segmentation. Thus, the FGF pathway plays a central role in orchestrating the dynamics of axial patterning and morphogenesis in humans. See also Yaman 2023.

### Experimental Models and Subject Details

### Cell Lines

All experiments were conducted using NCRM1 iPS cells with endogenous locus of HES7 tagged with Achilles and MESP2 tagged with mCherry (provided generously by the Pourquie Lab). Sex is not a relevant parameter for this study. iPSCs were cultured in 6-well tissue culture dishes treated for 1 hour with 1× diluted Matrigel (Corning) and supplied with mTeSR Plus media (STEMCELL Technologies). Media is changed with fresh media every two days. For routine culture, we passaged by washing with Dulbecco's phosphate buffered saline (DPBS) followed by ReLeSR (STEMCELL Technologies) treatment. Briefly, cells after washing with DPBS, cells are treated with ReLeSR for 30 seconds. Then ReLeSR is aspirated, and cells are incubated in the incubation chamber (37° C., 5% CO₂) for 5 minutes. Cells are rescued using 1 ml of mTeSR Plus. 25 μl of the cell suspension is added to a well of a 6-well plate coated with Matrigel. All cell lines used were routinely tested for mycoplasma contamination (Mycoplasma PCR Detection Kit, ABM Cat. No. G238). We used hiPSCs in accordance with approvals by Harvard University IRB (protocol #IRB18-0665) and Harvard University ESCRO (protocol E00065).

### Method Details

### Microfabrication and Soft Lithography

Briefly, round ADEX-50 dry film photoresist films (DJ Microlaminates) were laminated onto round 76.2 mm Si wafers (University Wafers) at 65° C. using a SKY laminator. Films were exposed through 8 μm-resolution photomasks (CAD/Art Services) to 365 nm UV light at an intensity of 25 mW/cm\hat{ }2 for 13 seconds using a UV lamp (Uvitron), baked at 80° C. for 15 minutes, and developed face down in cyclohexanone on a steel mesh without shaking for 5 minutes, followed by washing with acetone and isopropyl alcohol and drying with compressed air. Afterwards, masters were hard-baked on a hot plate at 200° C. for 1 hour and silanized for 1 hour in a vacuum chamber with 50 μl of trichloro(1H,1H,2H,2H-perfluorooctyl) silane. To cast PDMS, the elastomer base and curing agent (Dow SYLGARD™ 184, Ellsworth Adhesives) were mixed at a ratio of 1:9, degassed, poured onto masters, and baked at 80° C. for 1 hour. Stamps were covered with Scotch tape to prevent dust accumulation and cut using a scalpel.

### Glass Micropatterning

PDMS stamps were treated with 1× Matrigel overnight at room temperature. Stamps were washed with MilliQ water and dried using pressurized air. Stamps were brought into contact on the features-side to the glass coverslips (22 mm No. 1.5 square borosilicate glass coverslips (VWR)) and gently pressed with a tweezer to ensure contact. Stamps were removed after 30-60 seconds, and coverslips were submerged into PBS for storage at 4° C. until cell seeding.

### Generation of Human Axial Organoids

hPSC colonies on maintenance plate were washed with DPBS twice and incubated in Accutase for 10 mins in 37° C. incubators. Cells were resuspended as single cells at 1.5 M/mL density in mTeSR+ containing 10 mM Y27632. Micropatterned coverslips were mounted in 6-well glass bottom plates using silicone grease. 2 mL of the suspension was pipetted onto micropatterned coverslips and incubated at 37° C. incubator for 75 mins. Excess media and non-adherent cells were removed by aspiration, coverslips were washed twice with pre-warmed DPBS and replaced with mTeSR+ containing 10 mM Y27632. Cells were incubated overnight. On the following day, the media was replaced with mTeSR+ and cells were incubated overnight again. After 48 hours from initial seeding, cells formed confluent colonies on micropatterns. The differentiation was started by adding N2B27(DMEM/F12 with 1×N2, 1×B27, 1× penicillin/streptomycin, 1% NEAA, 0.5% GlutaMAX, 0.1% ß-mercaptoethanol, and 0.05% bovine serum albumin) media containing Matrigel (6%, v/v) supplemented with 4 mM CHIR99021 (CHIR), 0.5 mM LDN193189 (LDN) and 5 mM A83-01. The time of initiation of differentiation was denoted as timepoint 0. At 48 h, organoids with a single lumen on micropatterns could be observed. At this point, the media was replaced with E6, and organoids were removed from the surface using a cell scraper. Each organoid as moved to a well of a 96-well Low Bind plate containing 100 uL E6 and incubated overnight. At 72 h, E6 containing Matrigel was added to the wells, making the final Matrigel concentration 6% v/v. Organoids were incubated in this media for the following 48 hours. For extended incubation, additional 100 uL E6 was added to the wells.

### Sample Fixation and Immunostaining

Organoids were collected from 96-well plates and washed twice in DPBS before fixation in 4% PFA for 20 mins at room temperature. After the fixation, organoids were washed three times in PBS, then stored at 4° C. until the blocking step. Organoids were permeabilized and blocked in PBS with 0.3% Triton X-100 and 5% Normal Donkey Serum for 60 mins at room temperature. Primary antibodies were diluted in antibody dilution buffer and incubated overnight at 4° C. with gentle rocking. After the primary staining step, organoids were washed three times in PBS for 5 mins and incubated in Alexa-Fluor-conjugated secondary antibodies and DAPI diluted in antibody dilution buffer overnight at 4° C. with gentle rocking. Finally, organoids were washed three times in PBS and imaged. The following primary antibodies were used at the indicated dilutions: Rat anti-SOX2 (1:400, Thermo Fisher BTJCE), Rabbit anti-NCadherin (1:400, Cell Signaling D4R1H), Mouse anti-ZO1-FITC (1:800, Thermo Fisher 1A12), Goat anti-SOX1 (1:400, R & D AF3369), Goat anti-TBXT (1:400, R & D AF2085), Goat anti-TBX6 (1:400, R & D AF4744), Mouse anti-PAX6(1:400, BD Biosciences 561462), Mouse anti-β-catenin (1:400, BD Biosciences 610153), Rabbit anti-dpERK(1:200, Cell Signaling 9101). In the immunostainings for dpERK and β-catenin, the first two wash steps were skipped, and organoids were fixed in 96-well V-bottom plate by adding certain amount of 10% PFA to the media to have organoids in 4% PFA final concentration. In the case of dpERK staining, organoids were dehydrated in cold methanol for 10 minutes at −20° C. followed by three PBS washes. The rest of the standard protocol were followed for dpERK and β-catenin immunostainings.

### Hybridization Chain Reaction (HCR) of Organoids

HCR was performed on organoids following the previously published protocols. Briefly, organoids were fixed in 2% formaldehyde overnight. The next day, organoids were washed 2 times with PBST (0.1% Tween20 in 1×PBS) for 5 minutes and dehydrated with a series of graded methanol washes with 25%, 50%, 75%, and two times 100% methanol in PBST, 5 minutes for each wash. Samples were incubated overnight or until use at −20° C. The next day, samples were rehydrated through a series of graded methanol washes with 100%, 75%, 50%, and 25% methanol in PBST and two times with 100% PBST for 5 minutes each. Samples were treated with 25 μg/mL proteinase K for 4 minutes at room temperature and washed twice with PBST for 5 minutes each. Samples were refixed in 4% formaldehyde for 20 minutes at room temperature, then washed 3 times with PBST. Later, samples were washed with prewarmed PH buffer at 37° C. for 5 minutes. After the wash, samples were resuspended in PH buffer and incubated for 30 minutes at 37° C. Samples were incubated in 500 μL of PH buffer along with 4 pmol of the probe mixture overnight. On the next day, hairpin mixtures were prepared by bringing h1 and h2 of each hairpin to 95° C. for 90 seconds and leaving it at room temperature for 30 minutes. Two hairpin mixtures are then added to the amplification buffer with a final concentration of 48 μM. Samples were washed with probe wash buffer at 37° C. for four times, 15 minutes for each wash. Samples were then washed with 5×SSCT two times at room temperature, 5 minutes for each wash. Samples were washed once with amplification buffer for 5 minutes and resuspended in the hairpin mixture, incubated overnight at room temperature. On the final day, organoids were washed with 5×SSCT for 30 minutes at room temperature and nuclei were stained with washing organoids in the 1:1000 DAPI (2 μg/mL) three times, 25 minutes for each wash. Finally, samples were washed two times for 15 minutes with 5×SSCT at room temperature and imaged. HCR probe design was: FGF4 (Accession NM_002007), FGF8 (Accession NM_033163), WNT3A (Accession NM_033131), CYP26A1 (Accession NM_057157), ALDH1A2 (Accession NM_170697).

### Wide-Field Microscopy

Axial organoids at 72 h, 96 h and 120 h stage were imaged using a Zeiss AxioObserver Z1 inverted microscope in a humidified incubator (5% CO₂, 37° C.), with a Zeiss EC Plan-Neofluar 10×/0.30 NA objective. The 43 HE DsRed/46 HE YFP/47 HE CFP/49 DAPI/50 Cy5 filter sets from Zeiss were used. Images were acquired using an Orca-Flash 4.0 CMOS camera (Hamamatsu). The microscope was controlled using ZEN software. All images were analyzed using Fiji, Ilastik, MATLAB, Morgana or Python.

### Confocal Imaging of Fixed and Live Samples

Fixed samples were imaged on a Zeiss LSM 980 with Airyscan using a Zeiss 10×(NA 0.45) objective. Detection was performed on DAPI, AlexaFluor 488, AlexaFluor 568 and AlexaFluor 647 channels. A Z-stack with 1-micron intervals was acquired from the lower to upper apical surface of each organoid. After Airyscan processing, a maximum Z intensity projection was performed for visualization purposes. Nuclear segmentation is done and analyzed by using Arivis Vision4D.

### Time-Lapse Imaging of Organoids

For time-lapse imaging of organoids, the method described in generation of human axial organoids was followed until 70 hours of differentiation. Later, organoids were collected in a 5 mL Eppendorf tube and centrifuged at 200 rcf for 1 minute. Supernatant was removed and organoids were suspended in 2 mL of basal media DGIP (DMEM F-12 no phenol red, Glutamax, Insulin-Transferrin-Selenium, Penicillin-Streptomycin). Organoids were then transferred to 96-well glass bottom plates along with 10 μL of DGIP for each organoid, each well containing 50 μL of DGIP. Following the transfer of the organoids, 50 μL of ice cold DGIP containing 12% v/v Matrigel was added to each well, resulting 6% v/v final Matrigel concentration. The plate was then placed in humidified incubator for 2 hours, allowing the Matrigel to solidify. Later, the plate was moved to Zeiss AxioObserver Z1 inverted microscope in a humidified incubator (5% CO₂, 37° C.) and imaging was started immediately. Imaging was done using a Zeiss EC Plan-Neofluar 10×/0.30 NA objective. Images were taken every 15 min. The 43 HE DsRed/46 HE YFP filter sets from Zeiss were used. Images were acquired using an Orca-Flash 4.0 CMOS camera (Hamamatsu) with 2×2 binning. For perturbations, timelapse was paused and 20 μL of DGIP containing the respective small molecules or recombinant proteins without removing the culturing plate from the stage. In all perturbations with FGF4 ligand, the media is supplemented with 1 μg/ml heparin (Sigma Aldrich cat. no. H3393-100KU) The microscope was controlled using ZEN software.

### Micropatterning, Passivation of Glass Coverslips and Time-Lapse of Micropatterned Colonies

PDMS stamps were treated with 1× Matrigel overnight at room temperature. Stamps were washed with MilliQ water and dried using pressurized air. Stamps were brought into contact on the features-side to the plasma treated glass coverslips (22 mm No. 1.5 square borosilicate glass coverslips (VWR)) and gently pressed with a tweezer to ensure contact. 0.1 mg/ml PLL-g-PEG buffered with 10 mM HEPES was flown between the stamp and the coverslip surface and incubated for 30 min before removing the stamp. The coverslips were washed three times with PBS and the stamp was removed. hPSC colonies on maintenance plate were washed with DPBS twice and incubated in Accutase for 10 mins in 37° C. incubators. Cells were resuspended as single cells at 1.5 M/mL density in mTeSR+ containing 10 mM Y27632. Micropatterned coverslips were mounted in 6-well glass bottom plates using silicone grease. 2 mL of the suspension was pipetted onto micropatterned coverslips and incubated at 37° C. incubator for 75 mins. Excess media and non-adherent cells were removed by aspiration, coverslips were washed twice with pre-warmed DPBS and replaced with mTeSR+ containing 10 mM Y27632. Cells were incubated overnight. On the following day, the media was replaced with mTeSR+ and cells were incubated overnight again. After 48 hours from initial seeding, cells formed confluent colonies on micropatterns. The differentiation was started by adding N2B27(DMEM/F12 with 1×N2, 1×B27, 1× penicillin/streptomycin, 1% NEAA, 0.5% GlutaMAX, 0.1% ß-mercaptoethanol, and 0.05% bovine serum albumin) media containing Matrigel (6%, v/v) supplemented with 4 mM CHIR99021 (CHIR), 0.5 mM LDN193189 (LDN). The time of initiation of differentiation was denoted as timepoint 0. At 48 hours of differentiation, the plate was moved to Zeiss AxioObserver Z1 inverted microscope in a humidified incubator (5% CO₂, 37° C.) and imaging was started immediately. In the case of FGF4 perturbation, FGF4 was added to the media right before the imaging started. Imaging was done using a Zeiss EC Plan-Neofluar 20×/0.50 NA objective. Images were taken every 15 min. The 46 HE YFP filter sets from Zeiss were used. Images were acquired using an Orca-Flash 4.0 CMOS camera (Hamamatsu) with 2×2 binning.

### Single-Cell RNA Sequencing

Organoids were dissociated into a single-cell suspension using the Worthington Papain Dissociation System kit (Worthington Biochemical). Cells were counted on the LUNA-FX7 Automated Cell Counter (Logos Biosystems) using fluorescence detection for viability with an acridine orange/propidium iodide stain (Part No. F23011). After counting, the sample was loaded into Chip G per the user guide from 10× Genomics, and no alterations were made at any step of the protocol (Part No. CG000315). GEMs were formed targeting 10,000 cells and reverse transcription completed immediately after. The cDNA was cleaned from the GEM reagents, amplified for a total of 11 cycles and verified via TapeStation (Agilent Technologies). Amplified cDNA was diluted and ran on the 4200 TapeStation instrument using High Sensitivity D5,000 tape and reagents (Part No. 5067-5592 & 5067-5593). The amplified cDNA was fragmented, end repaired, and A-tailed followed by adaptor ligation, and PCR amplification for a total of 11 cycles with each sample receiving a unique set of dual indices (Part No. 1000215). Final libraries were diluted and ran using the High Sensitivity D5,000 tape and reagents (Part No. 5067-5592 & 5067-5593) on the 4200 TapeStation (Agilent Technologies). Libraries were quantified via Kapa qPCR using the Complete Universal Kit (Part No. 07960140001, Roche Sequencing Solutions) and the CFX96 Touch Real-Time PCR Detection System (Bio-Rad Laboratories). Libraries were sequenced on an Illumina NovaSeq instrument using the parameters outlined in the user guide (Read1: 28 bp, i7 index: 10 bp, i5 index: 10 bp, Read2: 90 bp). After sequencing and demultiplexing, the Cell Ranger count pipeline to align reads to the GRCh38 human reference genome and produce the associated cell by gene count matrix.

Processing of scRNA-Seq Data

Cells with 6,000 to 30,000 reads were subsampled to a obtain a read count of 6,000 for each cell. Sparse multimodal decomposition (SMD) was performed in Python on the subsampled count matrix. Top 150 genes with a highest z-score from SMD were filtered to remove genes with a log normalized expression mean or standard deviation less than 0.05, genes expressed in more than 90% or less than 1% of cells, and genes associated with the cell cycle, resulting in a list of 48 genes. In the subspace of these 48 genes, cells were hierarchically clustered, and 6 clusters were identified. Clusters were annotated manually based on differentially expressed genes. UMAP plots were generated using ScanPy.

Diffusion map analysis was performed using the DPT method in scanpy in the subspace of 48 genes. The root cell was selected to be a cell from the NMP cluster with the highest value along the first principal component from principal component analysis. To generate a pseudo-spatial gene expression heatmap, cells were ordered by pseudo-spatial index and their z-score normalized gene expression values were smoothed using a Gaussian filter with a kernel size of 50 for mesodermal clusters and 150 for neural clusters. Top 200 differentially expressed genes were selected for mesodermal and neural clusters, based on the difference of the highest and the lowest values of z-score normalized gene expression on respective clusters. Genes were then ordered by the pseudo-spatial position of their peaks of smoothed gene expression values. To generate pseudo-spatial gene expression plots for individual genes, a moving average filter was applied to log-normalized gene expression values on the pseudo-spatial axis was applied to mesodermal and neural branches with an averaging width of 201 cells for mesodermal clusters and 601 for neural clusters. Later, each gene expression was normalized based on the highest expression of the gene on the respective lineage.

Comparison of Cell Types with In Vivo Mouse Cells

To pre-process the mouse data set, we selected cells belonging to neural and paraxial mesodermal lineages in the mouse dataset. From this subset of cells, we removed doublets and cells with low read counts by selecting cells that had between 8000 and 12000 reads. We then normalized the read counts from each cell both in the mouse and human datasets. After log normalizing each dataset, we combined them (before which we found the corresponding homologous genes between mouse and human to map the mouse genes onto human genes). The resulting dataset consisted of 2376 mouse cells and 9096 human cells with reads from 7989 genes. From these genes we selected only the genes that had a high Z score (Z>1.5, 48 top genes) to reduce the gene expression space to those that were detected to be bimodally expressed. In this subspace, we performed hierarchical clustering, and found that cells belonging to the same cell identity clustered together. The cells from human and mouse were intermixed. Further, we annotated the cell types using marker gene expressions. We found that except neural progenitor cell type and mature somites, all cell types identified in the human dataset were present in the mouse tail bud. The absence of neural progenitors and mature somites in the mouse dataset was because these tissues are more anterior to the tail bud both in the embryo and in our organoids, therefore tail bud explant used for the single cell RNA sequencing did not contain these tissues in mouse. To visualize the cell types, we performed principal component analysis and plotted the cells in the first two principal component subspace.

Later, we isolated the cells belong to common cell types between these two datasets, namely Neuromesodermal Progenitors, Presomatic Mesoderm, Early Somite and Pre-Neural Tube. We looked for the highly differentially expressed genes in each cell type compared to the other cell types. By plotting the mouse and human cells in each cell type separately, we saw that the marker gene expressions were similar between these two species, validating that the mouse equivalent cell types of the cells in our organoids are present in mouse embryos.

We observed that the marker genes for NMP's are found to be mostly signaling molecules, consistent with the fact that NMP's are the signaling center that drives axial elongation and anteroposterior patterning. Therefore, we found the highly expressed FGF and WNT ligands in NMP's for both human and mouse cells and compared them to the other tissues. This analysis showed that WNT3A is specifically expressed in neuromesodermal progenitors and presomatic mesoderm cells in both human and mouse cells, which also explains the absence of canonical WNT ligands in the organoids of our companion paper due to the absence of NMP's and presomatic mesoderm cells.

### Simulations

For illustration, we posited a straightforward mathematical model based on our measurements to demonstrate how gradients of the diffusive FGF signals can drive anteriorly moving oscillations along the PSM. Most biochemical parameters are not known for human cells, so a detailed model is impossible. We extracted effective parameters for our model from our experiments and again emphasize that this model is for illustrating the idea and showing how simple models based on our assumptions show qualitatively similar dynamic behaviors to the organoids.

Our simulation consisted of n=110 cells initially, arranged along a line in one dimension. Each cell was 5 μm long. Thus, we set the initial length of the undifferentiated PSM tissue to be 550 μm long. We assumed that each cell had an internal oscillator with a base period of 300 min based on the experimentally measured period at the most anterior end of the PSM. Based on our experimental observation, the internal oscillator was assumed to be accelerated by the cell's FG activity. We assumed FGF profile is a function of distance from the posterior tip, x;

$$FGF ( x ) = {FGF}_{θ} ( 1 - exp ( - \frac{x}{{l}_{FGF}} ) )$$

where l_{FGF}=300 μm, estimated by dpERK measurements. Here FGF₀ was used to define a dimensionless variable and set it to the maximum level of FGF(x).

Next, we defined a short-range retinoic acid signaling, described by the function:

$$RA ( {x}^{′} ) = {RA}_{θ} exp ( - \frac{{x}^{′}}{{l}_{RA}} )$$

where x′ is the distance of a cell with presomatic mesoderm identity from the somite determination front, since retinoic acid is produced by somite cells, where RA₀ is a dimensionless variable and was set to 100, and l_{RA} was set to 10 μm. Given that retinoic acid inhibits FGF activity, we modified the FGF profile with an inhibitory retinoic acid term to define the FGF activity. Therefore, FGF activity at a distance from posterior tip, x, and from the somites, x′, is defined as:

$${FGF}_{activity} = \frac{FGF ( x )}{RA ( {x}^{′} ) + 1}$$

By using the measured FGF activity profile along the A-P axis and the measured frequency profile along the same axis, we inferred the function that maps the frequency of oscillation as a function of FGF activation:

(FGF)=1.391−2.619exp(−14.94*FGF/FGF₀)rad/hr

We modeled the growth of the organoid by cell division at the posterior tip. In the simplest model, we assumed this division rate to be constant and set it to 2.28 divisions an hour by using the measured growth rate. Below a threshold activity level of FGF, we postulated that the presomatic mesoderm would differentiate into somite cells. We calculated this activity level by using the FGF activity profile function, and the mean length of the presomatic mesoderm measured and set to be FGF_{diff}=0.08*FGF₀. Cells in the model that passed this FGF threshold became somite and started expressing MESP2 when their internal oscillation phase was between (0,π/2). This assumption was to model the activation of HES7 and MESP2 simultaneously since both are the NOTCH pathway target genes.

External activation of the FGF pathway simulated by the FGF ligand was incorporated into the model by making the FGF activation along the entire PSM uniform and set to the maximum observed activation level, FGF₀.

Using these assumptions, we simulated the model using MATLAB. We set the initial phase of the internal oscillator to zero, therefore initial oscillations were synchronous. Over time, we observed a growing organoid with anteriorly traveling oscillations and a posteriorly moving somite determination front consistent with the experiments. When we stimulated this model with uniform FGF profile by setting FGF(x)=FGF₀ at t=25 h, the entire PSM oscillated in synchrony as observed in the experiment (data not shown), and the determination front slowed down, consistent with the experiment. In accordance with the two-phase model, while unperturbed control organoid, in which there are traveling HES7 waves, showed alternating MESP2 expression in a single somite segment, addition of FGF4 disrupted this alternating expression (data not shown), due to synchronous HES7 oscillations.

### Quantification and Statistical Analysis

### Analysis of Time-Lapse Imaging Data and Generation of Kymographs

Organoids were segmented using Ilastik on the phase contrast images. Fluorescence images were corrected for nonuniform illumination and autofluorescence of the media was subtracted by calculating the mean signal of the empty area for each image. Segmented organoids were rotated on the major axis and fixed on the posterior end. Signal for each pixel on the anteroposterior axis was calculated by averaging all the pixel values on the line perpendicular to the major axis on the respective position. Kymographs for HES7 and MESP2 signal were generated by using the calculated signal on the anteroposterior axis for each timepoint.

Length fold change was calculated by finding the length of the major axis of each organoid and dividing them to their initial length. The length of the somatic mesoderm of each organoid was calculated by manually thresholding and binarizing each MESP2 kymograph and calculating the length of the line on the space axis corresponding to each timepoint on the binarized kymograph. The length of the PSM is calculated by finding the distance from the posterior end to the position, of somite determination front, which is the most posterior position of the somatic mesoderm on the major axis. Fold change of the PSM length was calculated by dividing the PSM length at each time point by the initial PSM length for each organoid. Segment sizes were calculated using the length of the somatic mesoderm. First, the change in the length of the somatic mesoderm between each timepoint was calculated. The timepoints corresponding to the troughs of the somatic mesoderm length change was detected. The difference of the length of the somatic mesoderm between troughs was calculated as segment sizes.

### Quantification of Phase and Frequency Profile on Anteroposterior Axis and Amplitude of Oscillations

For phase analysis, first oscillating region of each organoid was detected using HES7 kymographs by calculating the frequency with highest amplitude of the Fourier transform of the time series of each position on the anteroposterior axis. Positions with peaks between 0.21 and 0.24 h⁻¹ were considered to be oscillating around the segmentation clock frequency. After cropping the oscillating region from kymographs, we used Hilbert transform following a detrending and local renormalization algorithm, following a similar approach to a previously published algorithm. First, a moving average filter was applied on the space axis of kymographs with 26.66 um window. To subtract the trend in the time axis, a moving average filter with a 6-hour window was applied to the kymograph and the resulting kymograph was subtracted from the original. The time series for each anteroposterior position was then divided by a standard deviation filter with a 6-hour window. Later, phase of each position was obtained by using Hilbert transform on the time series between 38-43 hours of the timelapse, corresponding to 4th oscillation after the small molecule perturbations. For calculating the frequency profile on the anteroposterior axis, the change in the phase was calculated between consecutive timepoints for each position during the time-lapse, and the period was calculated using this value. The position is calculated from the anterior tip.

The amplitude of the oscillations was calculated using the detrended kymographs. The mean signal was calculated for the posterior part of each organoid, corresponding between 26-133 um region from the posterior end. Peaks of the oscillations after the small molecule perturbations was detected. The signal of the fourth peak following the perturbation was normalized by the signal of the first peak following the perturbation.

### Example 4. Microcontact Printing Methods

### 1. Microcontact Printing Using PDMS Stamping on Coverslip Substrate:

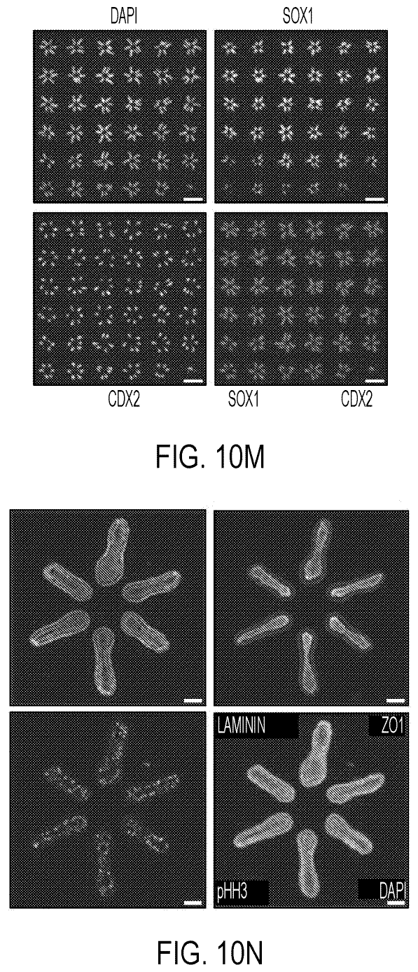

Microcontact printing (CP) is a widely used technique for transferring a monolayer of protein onto a substrate, typically a glass surface. The process involves creating a stamp of cured polydimethylsiloxane (PDMS) with specific features designed for the desired protein pattern. This stamp is typically fabricated through photolithography on a silicone wafer, resulting in a high-resolution and reproducible stamp. In this Example, the PDMS stamp containing positive features is then coated with Matrigel, which adheres to the substrate due to non-specific adsorption or specific binding interactions. The protein not on positive feature will then not contact with the surface, leaving only the protein adhered to the desired pattern on the substrate. The induced pluripotent cells (iPSC) will then be attached to the printed Matrigel on the surface for further culture. See FIG. 12.

### 2. Advantages of PDMS Microwell Chips Over Coverslips in Microcontact Printing.

Through the well-established photolithography technique, an AutoCAD-generated design can be readily transferred onto a silicon wafer, which can then be used to fabricate PDMS chips with simplicity and efficiency.

Such PDMS chips can be a great replacement for conventional coverslips for microcontact printing, as it enables easy design and allows for various customization, such as wells in this case. Based on the design, the wells on the PDMS chips can be used for morphogen-coated bead capitulation, making it a morphogen source to generate local gradients across the printed organoids through a special microcontact printing process.

### Fabricate Microwells on PDMS to Capture Morphogen-Coated Beads:

The mold of the device is fabricated with the traditional photolithography process on a 3-inch silicon wafer. The height of the feature is made into 100 um by spin coating a SU-8 2100 photoresist film onto the wafer, followed by the subsequent soft bake, edge bead removal, UV exposure, post-bake, PGMEA development, dehydration bake, and salinization steps. The PDMS base and curing agent are mixed at a 10:1 ratio and poured onto the mold. To ensure a thin layer of PDMS microwell chips, the silicon wafer was submerged with mixed PDMS, it was desiccated to remove air bubbles, and then a salinized glass slide was gently placed on the mixed PDMS to create a uniformly spread 1 mm thin film in between.

After peeling the PDMS thin film off the master and the glass slide, it was placed onto a plasma-activated 22 mm*22 mm glass coverslip. The coverslip enables the PDMS film to flat during the experiment and can be attached to the plastic petri dish with vacuum grease, thereby preventing flotation. While combining the PDMS film with the coverslip, two 3 mm*22 mm*4 mm pieces of flat PDMS are also bounded onto the edges of the coverslip in parallel to the potential water channel. Such flat PDMS pieces are made by curing a mixture of 25 g of PDMS in a 10 cm petri dish and cutting it from the dish with the target shape. These two “walls” will form a small chamber by putting a glass coverslip on the top during the media change in cell culture to prevent the surface from getting dry on such a hydrophobic surface, preventing any subsequent stem cell death.

3. Coating of Morphogen Beads and Deposition into Microwells:

The heparin-agarose beads (Sigma) were initially washed three times with 1 ml PBS by centrifuge and supernatant removal. Then the BMP4 and fluorescent conjugated BSA were added to a tube of beads for the desired beads concentration. The tube will be rotated in the 4° C. cold room overnight.

After the overnight preparation, a small volume of beads is collected from the tube and washed three times with PBS by centrifuge and supernatant removal. The beads are then suspended into a 50 ul PBS and uniformly added onto the device with organoids and wells. Then they will be moved into the nearby wells under the microscopy with a capillary glass tip controlled by the pressure-induced microinjection machine. The capillary glass tip is fabricated by pulling a capillary tube to form an ultra-fine tip, followed by polishing to make the tip with desired diameter, in this case, 100 um for injection and 120 um for beads removal. After washing several times thoroughly with DPBS to remove any floating beads, N2B27 basal media was added back and make a final cleanup with 120 um glass capillary tip to ensure no extra beads are left on the device other than the wells.

### 4. Challenges in Microcontact Printing on PDMS and the Development of Humidified Microcontact Printing:

In contrast to conventional CP, the experimental design involves printing on a hydrophobic PDMS surface using stamping. In CP, the protein to be printed is typically absorbed onto the hydrophobic PDMS stamp and then transferred onto a hydrophilic surface, similar to inking. The investigation conducted by Chen et al. suggests that this transfer process can be characterized as a function of surface energy, with printing occurring from the low surface energy side to the high surface energy side. Their findings reveal that a decrease in the surface energy of the target surface results in diminished transfer from PDMS to that surface. In other words, as the surface becomes more hydrophobic, achieving protein transfer becomes more challenging.

In this approach to achieving printing from a PDMS stamp to a PDMS device, the concept of humified microcontact printing (HμCP) as defined by Ricoult et al. was utilized. The main idea behind this approach is to utilize flowing water to transfer the pattern onto a high energy surface, which in this case is the PDMS microwell chip. To prevent the condition of the transfer occurring between PDMS surfaces with similar surface energy, a high energy PDMS stamp is used as an intermediate stamp during the process. This high energy PDMS stamp is created by exposing the PDMS surface to plasma treatment, which generates polar functional groups such as silanol, carbonyl, and carboxyl groups.

In accordance with the concept introduced by Ricoult et al., the flowing water can form hydrogen bonds with the oxygenated functional groups on the high energy stamp. However, in PDMS, the methyl groups rotate around the backbone, effectively shielding the Si—O groups and preventing direct hydrogen bonding between PDMS and water molecules. This facilitates the transfer of the desired molecules, such as Matrigel in this case, from the high energy PDMS surface to the PDMS microwell device side. The high-energy PDMS stamps can effectively ink the Matrigel onto their positive features via CP from a low-energy Matrigel-coated flat PDMS stamp.

### Printing on PDMS to Position Organoids

### Stamp Preparation:

In AutoCAD, an array of circles was designed with dimensions of 8*8, each containing sets of 4 circles with a diameter of 150 μm. The sets of 4 circles are spaced 2000 μm apart. Subsequently, a 3-inch silicon wafer will be coated with a layer of 50 μm SU-8 3050 photoresist for desired patterning through photolithography. After post-bake, PGMEA development, dehydration bake, and salinization steps, a 10:1 PDMS mixture can be poured into the master to create a simple stamp for printing. A flat PDMS stamp is made by pouring 10:1 PDMS onto a 10 cm petri dish and curing it at 80° C. overnight, followed by cutting for use.

### Microcontact Printing (μCP)

Initially a drop of 5× Matrigel was added onto flat PDMS stamps, followed by a plasma activated coverslip, and allowed to coat at room temperature overnight. Afterward, the coverslip was removed, the stamp was washed with MilliQ, and air dried for 30 seconds. Then the plasma treated PDMS stamp with the desired feature will be pressed onto that initial flat PDMS for 5 minutes, allowing the positive feature to be coated with Matrigel.

### Humified Microcontact Printing (HμCP)

To transfer the Matrigel onto the high surface energy PDMS device, the coated low energy PDMS stamp were put onto the device and aligned the print in the center of the wells visually. Subsequently, a weight of 20 g will be placed onto the stamp, and 4 ml 37° C. prewarmed MilliQ water will be injected in between. The entire setup is then transferred to a 37° C. incubator for 30 min. After such incubation, the remaining water was gently aspirated and was the heavy weight was gently removed to prevent sliding between the stamp and the PDMS microwell substrate. For further cell seeding, 2.5 mL warm mTeSR (+) media supplement with 1× penicillin-streptomycin (Pen-Strep), 2.5 ug/ml Amphotericin B, and 10 μM Rock Inhibitor was added.

5. Design and Fabrication of 2-Layer Stamp with Water Channel (Enhance Diffusion of Water Molecules):

However, the laterally diffusive water molecule from neighboring water is limited in diffusion length, as demonstrated by Ricoult et al. The diffusion is confined to a distance of approximately 300 μm, which means that if the distance of the central parts of the feature from water exceeds 300 μm, it may affect the transfer of the Matrigel to that area and bring the defect in the printing. In that case, vascularized water channels near one or more (or all) feature on the PDMS stamp will be included to ensure the diffusive water molecules from those channels can reach the center of the feature to get every Matrigel transferred.

### 2-Layer Stamp Fabrication

The 2-layer stamps were fabricated using a three-layer silicon wafer master. A 3-inch silicon wafer was initially coated with a 50 μm thick layer of SU8-3050. Subsequently, the 8*8 array consisting of sets of 4 circles, each with a diameter of 150 μm, was patterned onto the SU8-3050 layer using soft lithography. Following a post-exposure baking step, a second layer of 50 μm SU8-3050 was spin-coated onto the first layer. The vascularized channels were designed to be fully aligned with the array features, such that each circle on the array will have at least one channel in 50 μm proximity. The width of the vascularized channels will then be at least 300 μm. Such vascularized channels will be aligned with the first layer feature on the wafer using a microscope and then exposed to UV light again to polymerize the second layer.

After the post-baking step, the development agent Propylene glycol methyl ether acetate (PGMEA) was employed to effectively remove all unexposed SU-8 from both layers on the silicon wafer. The silicon wafer was subsequently dehydrated by placing it on a 120° C. hotplate for 2 hours. Following dehydration, the wafer was transferred to a desiccator, with two drops of silane added to a glass slide nearby. The desiccator was then placed in a vacuum environment for overnight evaporation. Subsequently, a mixture of PDMS at a ratio of 10:1 was poured into the prefabricated silicon master. After desiccation to remove air bubbles, the mixture was cured at 80° C. oven overnight. Finally, the stamp was gently peeled off from the master to obtain the desired stamp structure.

### 6. Cell Seeding, Culturing, and Folding on PDMS Microwell Chips:

The human induced pluripotent stem cells (hiPSCs) were washed once with DPBS and subsequently incubated in Accutase for 10 minutes at 37° C. in a cell culture incubator. The cells were then dissociated into single cells, centrifuged, and resuspended in mTeSR+ medium supplemented with Pen-Strep, Amphotericin B, and Rock Inhibitor. The single cells were then seeded onto the printed PDMS stamp at a density of 3 million cells per well. The PDMS microwell chips containing the seeded cells were subsequently transferred to an incubator and kept for 75 min. After the incubation, the non-adherent cells were gently aspirated and thoroughly washed multiple times with DPBS until no floating cells were observed. Then a coverslip as placed down onto the walls and washed three times with mTeSR+ medium containing Pen-Strep, Amphotericin B, and Rock Inhibitor. The coverslip on the “walls” could help the chamber remain hydrated throughout the washing process.

After overnight incubation, the media was washed three times with mTeSR+ medium, including Pen-Strep and Amphotericin B. A coverslip will always be on the “walls” during aspiration. Subsequently, the PDMS chip will be moved to the refrigerator for 10 min, and then add a silanized coverslip on the “walls”. 1 mL mTeSR+, including Pen-Strep and Amphotericin B containing Matrigel (4%, v/v), will be added to the well to fold the cells after aspiration. Afterward, they will be chilled in the refrigerator for ten more minutes before moving to the incubator.

### Example 5. Delivery of Morphogen Signals Using Controlled Signal-Coated Bead Positioning to Refine Signaling Gradients

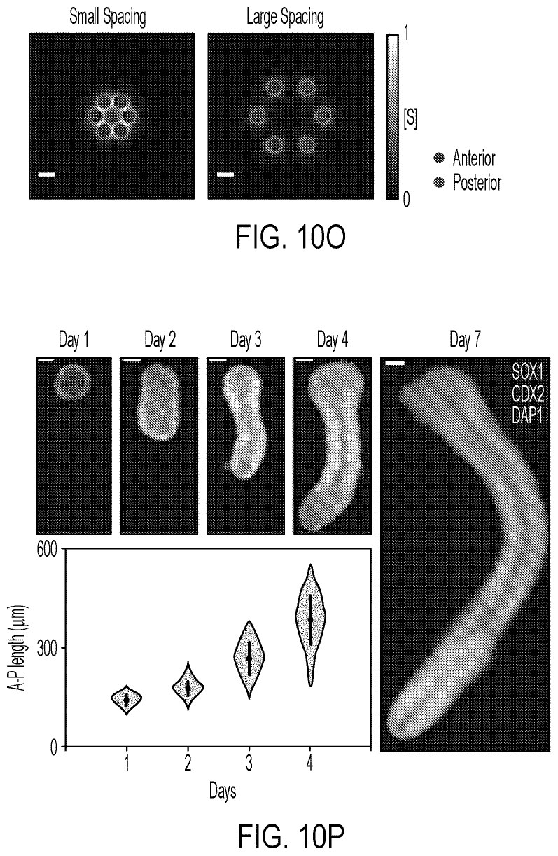

This Example describes a method for controlling signaling gradients. A polydimethylsiloxane (PDMS) substrate was fabricated with wells to hold signal-secreting beads at desired locations using photolithography. Microprinting was used to stamp ninety-six (96) 150-micron circles of extracellular matrix protein on the hydrophobic PDMS thin film above (FIGS. 13A-13C). Pluripotent stem cells were dissociated into single cells and seeded onto the coverslip with the patterned 150-micron matrix spots at a density of 3 million cells per well. Media containing extracellular matrix protein Matrigel (4%, v/v) was added to fold the cells on each matrix spot into a cyst with a single epithelium enclosing a lumen. Heparin-agarose beads (Sigma) were exposed to signal ligand (e.g., BMP4) and fluorescent conjugated BSA overnight at 4° C. in the cold room. The beads were washed and suspended in 200 μL of N2B27 basal media. The beads were deposited into wells at desired locations next to stem cell cysts using a pressure-induced microinjection 100 μm capillary glass tip. This was done at high throughput, exposing every one of the 144 cysts per coverslip to different beads at different distances from the cysts. The signal-secreting beads generated signaling gradients (FIGS. 13A-13C). Beads coated with different signals could simultaneously be placed in different wells to generate more complex signaling gradients.

While this example describes a microprinting method using PDMS as the substrate, any biocompatible substate can be used, for example, polystyrene, poly-1-lysine, fibronectin, collagen, gelatin, Matrigel, polyethylene glycol, or silicone.

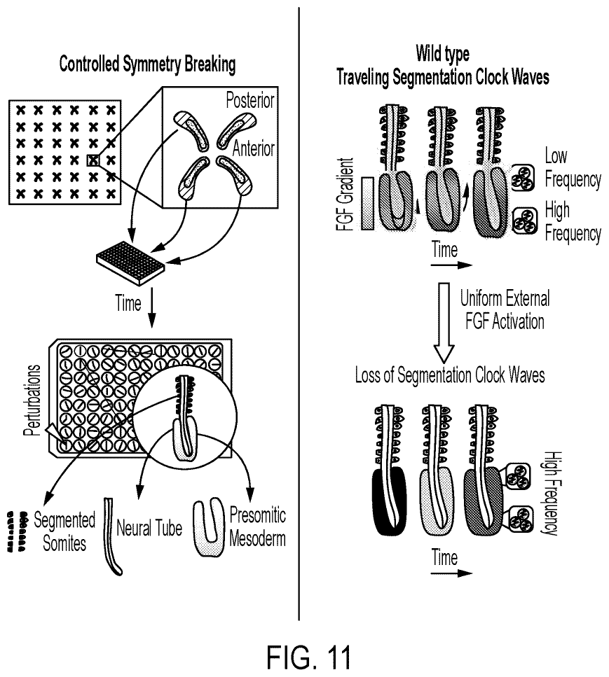

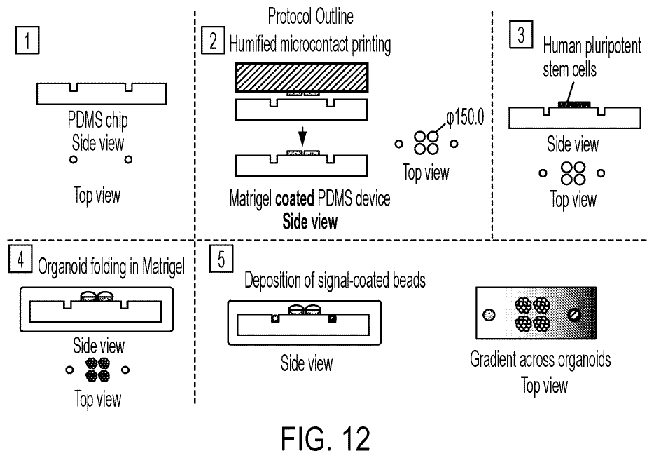

For real-time monitoring of organoid patterning, we chose two genes based on in vivo gene expression analysis from first-trimester fetal tissue: OTX2 (initially expressed throughout the forebrain, becomes gradually restricted to the midline dorsally, and in the ventral forebrain) and LHX2 (localized to the hem, cortex and ventral forebrain). Using CRISPR, we built a single-reporter human embryonic stem cell with m-Citrine tagged to OTX2 at the endogenous locus and, similarly, a double reporter line with OTX2-mCitrine and LHX2-iRFP. We ascertained that the lines differentiated exactly as the parental WA01 hESC line. We printed hESCs from these two lines onto hexagonal patterns as in Example 2 (see also Anand 2023) above on PDMS with prefabricated wells positioned at the outer edges of the pattern at 100 μm from each organoid. We exposed the resulting cysts to forebrain induction media. After 2 days of differentiation, we placed BMP-coated beads (pre-exposed to 50 ng/ml BMP4 as outlined above) in the wells. The cysts differentiated as in Example 2 (see also Anand 2023), with the epithelium closer to the bead differentiated into ectoderm, while that farther from the cyst differentiated into the neural epithelium. In four days, the neural epithelium folded as in Example 2 (see also Anand 2023), and the neural plate boundaries merged to recapitulate neural tube closure. We removed the organoids from the coverslip after the four days of BMP bead exposure, moved them individually to 96 well plates, and continued forebrain differentiation without any externally added signals for 30 days. OTX2-mCitrine expression gradually localized to the midline by day 10, at which point, LMX1A (hem, ChP marker) was similarly localized. In thirty days, TTR, the marker of the ChP), was expressed at the midline. Both PAX6 and LHX2 expression showed that the cortex is patterned more distally from the midline (FIG. 14). Simultaneous positioning of SHH on the inner edge and BMP4 on the outer edge during the first four days resulted in dorsoventral patterning (FIG. 15).

### Example 6. Validation of the In Vivo Relevance of Organoids by Perturbing Critical Genes That should Affect the Patterning and Function of the Choroid Plexus, Hem, and Cortex

The organoids patterned using BMP gradients in Example 4 show signs of a functional midline and hem. These organoids showed a stable lumen that increased in volume with time (FIGS. 14 and 15), suggesting that the midline was capable of making fluid to fill the lumen. We expect the fluid to have the correct CSF composition. The Cajal-Retzius (CR) cells secreting the glycoprotein REELIN that emerge from the cortical hem consistently appeared at the basal surface of the developing organoid's cortex (data not shown) in the presence of BMP gradients. The distal PAX6+, LHX2+ cortical progenitors in the organoids generated lower layer neuronal markers TBR1 and CTIP2 over fifty days, demonstrating their developmental potential. In contrast, in the absence of a BMP signal, the internal lumen of the organoids collapsed over time (data not shown), and they did not possess any CR cells.

We identified critical genes expressed in 4-week-old human fetal tissue and in vitro single-cell data known to affect the dorsal forebrain patterning and function in vertebrates, including. (i) BMPR1A, whose knockdown in the dorsal forebrain abrogates the patterning of the choroid plexus (ChP) and TTR expression at the midline in mouse, (ii) BMPR1A/BMPR1B double knockdown eliminates both the ChP and hem without affecting the cortex or ventral fates in mouse, (iii) Na+K+ ATPase ATP1A1A knockdown causes lumen collapse by abrogating ChP function in zebrafish, (iv) Na+K+ ATPase ATP1A3A has the opposite effect driving ventricle dilation, (v) knockdown of EMX1 eliminates the hem (and hence the primary source of Cajal Retzius cells) and expands the roof plate, (vi) each of PAX6 and LHX2 knockdown eliminates the cortex. None of these genes are known to be required prior to forebrain development.

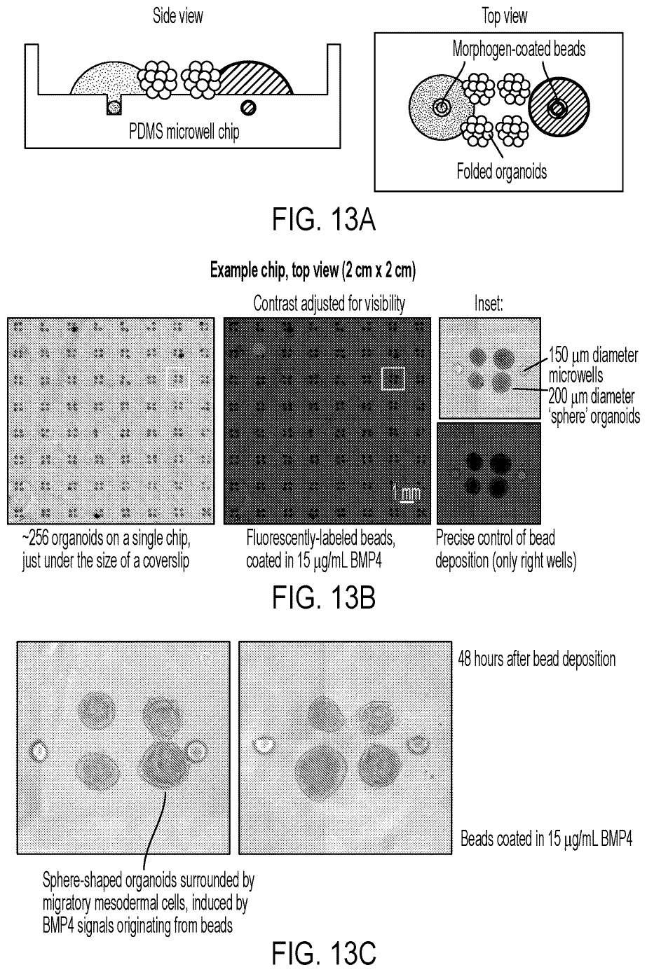

We generated dCAS9-RFP (CRISPRi) and a doxycycline inducible-dCAS9-RFP (iCRISPRi) reporter lines in the OTX2-mCitrine/LHX2-iRFP background hESC line by targeting dCAS9 driven by either a CAG promoter or a doxycycline-inducible promoter to the AAVS1 locus. For the inducible promoter, we used the XLone inducible expression system. Twenty-four hours before cell sorting, the iCRISPRi (inducible CRSPRi) population was treated with 2 μg/ml doxycycline to induce expression of RFP. Individual clones were genotyped using PCR and Sanger sequencing. Genotyped clones were assessed for knockdown efficiency using lentivirus carrying gRNA sequences against the surface markers CD55 and CD151 (FIG. 16). Viral Constructs. Two guides against the target gene were cloned into a 3rd generation lentiviral transfer pLKO5 backbone with an EF1-alpha promoter driving a fluorescent reporter (mCerulean or GFP). The guides were each driven by a U6 and H1/S7K hybrid promoter. The CRISPRi lines without or with only CFP-carrying lentiviruses differentiated normally.

### Example 7. A High Throughput Perturbation Platform

We developed protocols to produce a high number of lentiviral particles in just 50 uL, sufficient to transduce a hundred thousand stem cells. We also developed protocols to increase viral infection efficiency to over 92% of the cells. Consequently, we could achieve a 32× reduction in time and cost compared to standard viral concentration protocols that yield comparable transduction efficiencies in hESCs. The protocol is outlined in the experimental design below. Targeted viral delivery and transduction at high throughput and penetrance A single coverslip contained 144 organoids arranged in 24 independent hexagons, with each hexagon having a central organoid (which is not considered). We devised a transient well system to isolate each hexagonal set of cysts from the others using removable PDMS wells (FIG. 17). In each well, we could deposit a single type of virus carrying a plasmid with two guides against a target gene and a promoter driving CFP. At least 92% of the cells in each organoid were infected by the viral vector carrying the specific lentiviral guide (FIG. 17A). The cross-contamination between wells was less than in 0.001% of the cells. Thus, on a coverslip with 144 organoids, we could perform 24 distinct penetrant perturbations in six replicates. Small media volumes and less time per experiment reduced costs by 80-fold, allowing perturbations on scales not previously possible.

We will design two guides against each of the top 198 genes predicted to be important, driven by the U6 and H1/S7K promoters, and clone them into 3rd generation lentiviral transfer pLKO5 backbone with EF1-alpha promoter driving mCerulean. Guide scaffolds will contain capture sequences in the hairpin for direct capture by 10× sequencing.

HEK cells will be seeded in a 96-well plate and incubated at 37° C. and 5% C02 and grown to 95% confluency. Two master mixes: (i) third-generation lentiviral packaging plasmids in jetPrime buffer, (ii) jetPrime reagent diluted 1:17 in jetPrime buffer, will be deposited into a 96-well PCR and incubated. Post-seeding, the HEK cell media will be replaced with resuspended transfection mix. After incubation, the plasmid-carrying media will be removed and replaced with fresh media. The plate will be incubated again until transduction.

We will print 24 evenly spaced hexagonal patterns on a coverslip. Each hexagonal pattern will involve 150 μm diameter circles of basement membrane protein in hexagons with 250 μm center-to-center spacing and an additional circle in the middle of the hexagon. We will place a removable PDMS well system on the coverslip to isolate one hexagonal pattern from the other (FIG. 17A). We will use the dCAS9-RFP (CRISPRi) and a doxycycline-inducible dCAS9-RFP (iCRISPRi) hESC lines in the OTX2-mCitrinelLHX2-iRFP background. hESC single cell suspension from each line in mTeSR(+) will be pipetted into a 96-well mixing plate. Viral growth media will be drawn up and mixed with the single-cell suspension. One of the 24 wells will contain viruses carrying a scrambled guide with no gene target and serve as a negative control. The mixed cell suspension from 24 wells will be immediately deposited onto 24 hexagonal patterns insulated from each other by the PDMS well system. The cells will settle onto the extracellular matrix protein patterns on the coverslip and be infected by the specific virus. The infection efficiency will be monitored by CFP fluorescence imaging as the viral constructs drive CFP expression. We expect a transfection efficiency of at least 92% based on multiple replicates in preliminary work (FIG. 17A). The infected cells will form a single epithelial layer of cells. The deposited cells will be incubated. After incubation, virus-containing media will be removed and replaced with buffer. Pluripotency media will be added and incubated for 24 hours. Media will be changed again, and the PDMS well system will be removed for ease of maintenance and assaying.

Extracellular matrix protein will be added to fold the infected epithelial cells on each pattern into a cyst and then differentiate them precisely with two days of forebrain induction media and two days of BMP treatment. For inducible dCAS9-iRFP, we will add doxycycline to knock down the desired gene within 24 hours of dox addition based on preliminary data.

We expect the epithelium of the unperturbed scrambled guide control cysts to be patterned into the ectoderm and neural plate with a neural plate border between these two tissues. Over the next two days after BMP removal, in the control unperturbed cysts, we expect the neural plate to fold and the neural plate borders to fuse to enclose a lumen. At this time, we will fix the organoids on the coverslip. We will antibody stain them for cell fate specific cadherins ECADH, NCADH, and the tight junction markers ZO-1, and image under a confocal microscope to assay for defects in patterning of the neural plate (NCADH+), ectoderm (ECADH+), neural plate boundary (ECADH+, NCADH+), and assay for epithelial integrity and closure using ZO-1. We will quantify the organoid size, lumen size, interface length between the ECADH+ and NCADH+ region, and epithelial integrity at the point of closure and compare these quantities with the scrambled guide control organoid, which will behave as wild type. Preliminary Results show that morphogenesis is perturbed in ZIC2, ZFN521, SOX11, and RHOA knockdowns, as predicted by the computational results (FIG. 17B).

We will collect all organoids in which transcription factors were perturbed. Following preliminary work, we will dissociate these organoids at the end of the differentiation protocol using gentle dissociation reagents to obtain 10,000 single cells per perturbed organoid. We will pool cells from all these organoids and prepare single-cell libraries for sequencing using standard 10× Chromium protocols, including primers for the 10× adapters in the stem loop of the guide RNAs. We will use HiSeq, as in preliminary work, to yield half a million reads and levels of over 7000 genes per cell. We will use our bowtie2 based computational pipeline for read mapping. The reads will provide both the transcriptome of each cell, and the 10× adapters will generate the guide sequence in that cell exactly as in established pooled methods, identifying the gene that was perturbed in that cell. The guide sequence will thus map cells back to organoids in which that gene was perturbed.

We will have established a platform for targeted lentiviral perturbations, allowing 24 perturbations per coverslip of organoids. We will have perturbed 198 genes implicated by the computational analysis and identified patterning and morphogenetic defects through imaging. The methods developed here will be widely useful to the community for studying in vitro human development.

### Example 8. Genetic Investigations of Human Neural Fold Morphogenesis

As discussed herein, due to ethical constraints, there is no in vivo model for human embryonic development. The most widely used model of mammalian embryonic development is mouse, but mouse morphological development is fundamentally different from human and utilizes a different genome. Recent developments in human stem cell-derived organoids allow us to recapitulate some aspects of human morphogenesis in a human genetic system. In order to screen for genetic factors controlling tissue-wide processes like morphogenesis, we need to develop organoid systems in which an entire organoid is perturbed by a single perturbation (rather than, for example, pooled perturbations).

We have developed a micropatterned organoid chip with 24 sets of organoids that allows us to screen morphogenetic genes at scale. Each set of organoids on this chip is perturbed at high efficiency with a single gRNA, meaning that almost all cells of a given organoid experiences the same genetic perturbation. Thus, we can test the role of each gene on tissue-wide phenomena. So far, we have shown that several mouse genes that cause exencephaly also cause closure defects in the human model.

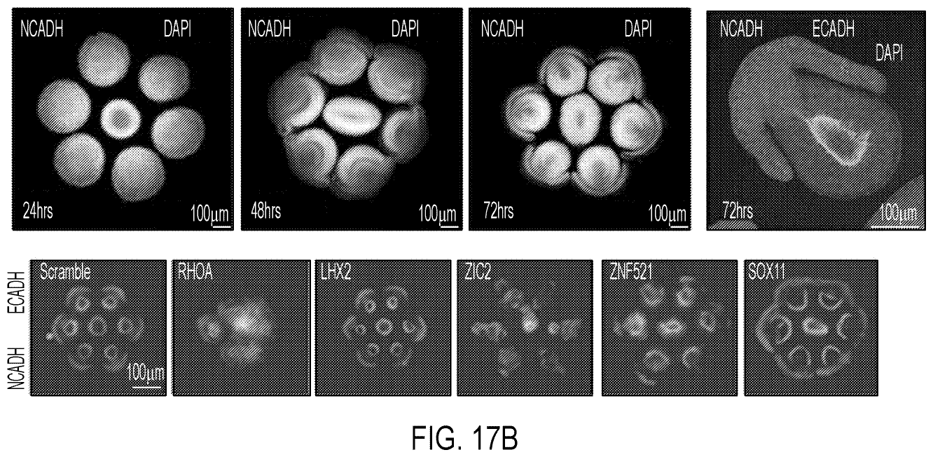

As shown in FIG. 20, application of high-titer lentivirus led to high and consistent transduction efficiency across gRNAs. The gRNA virus carried a CFP marker to measure transduction efficiency.

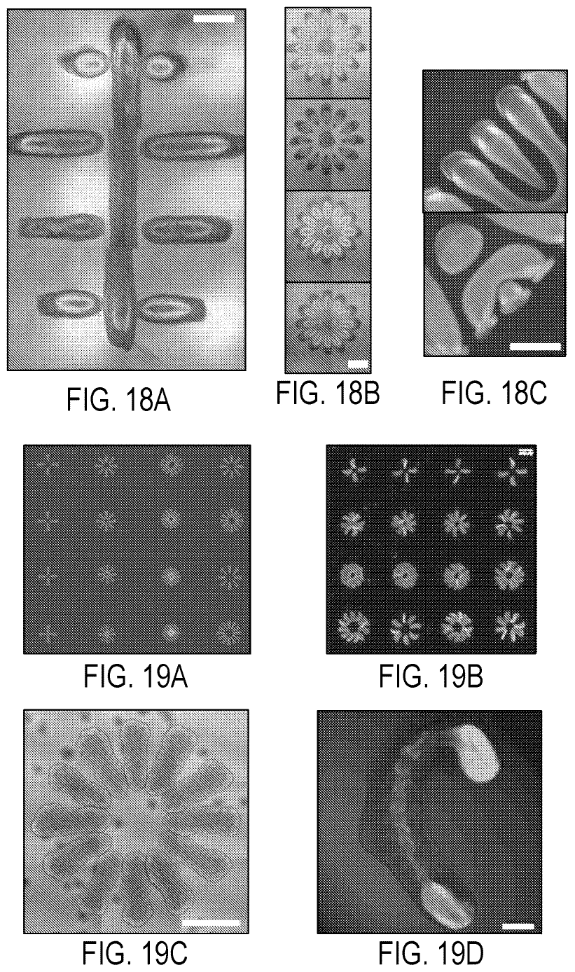

As shown in FIGS. 21A and 21B, our analysis of lentiviral transduction efficiency by pixel analysis and nuclear OCT4 knockdown show high efficiency (87%-88%).

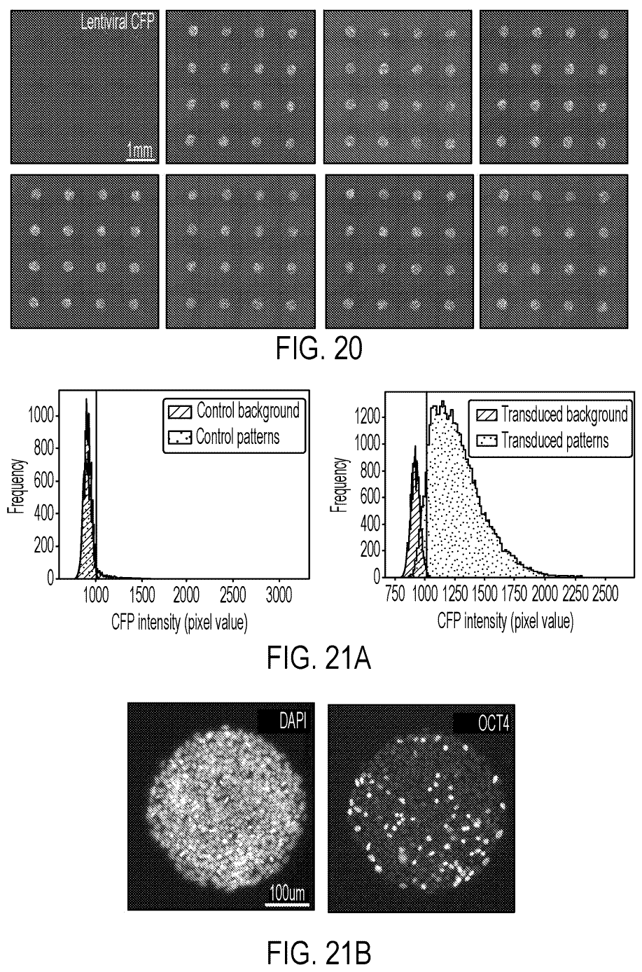

The data provided in FIG. 22 shows that knockdowns of several genes (CSNK2A1, SPINT2, and SNX13) known to cause exencephaly in mouse showed folding defects in our human model. All organoids were phenotyped with an anti-ZO1 to and epifluorescence microscopy to visualize apical lumen morphology. The central neural lumen was intact, circular, and continuous in both the no-virus control and the scramble gRNA control. Knockdowns of CSNK2A1 caused a loss of neural lumen formation, and knockdown of SPINT2 and SNX13 showed abnormal lumen (perhaps double lumen) morphologies.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

What is claimed is:

1. A method of using a trained machine learning (ML) model to identify a spatial arrangement of stem cell aggregates, the spatial arrangement for promoting development of the stem cell aggregates into target patterned tissue, the method comprising:
using at least one processor to perform:
accessing the trained ML model, the trained ML model being trained to predict, using a feature vector generated for an input spatial arrangement of the stem cell aggregates, a patterning metric associated with the input spatial arrangement; and
identifying a spatial arrangement of stem cell aggregates that optimizes the patterning metric by using a stochastic optimization technique in which different spatial arrangements of stem cell aggregates are evaluated using their respective patterning metrics as predicted by applying the trained ML model to feature vectors generated for the different spatial arrangements.

2. The method of claim 1, wherein the different spatial arrangements include a first spatial arrangement, wherein applying the trained ML model to feature vectors generated for the different spatial arrangements comprises:
generating a first feature vector for the first spatial arrangement; and
providing the first feature vector as input to the trained ML model to obtain corresponding output including a first patterning metric for the first spatial arrangement.

3. The method of claim 2, wherein generating the first feature vector for the first spatial arrangement of stem cell aggregates comprises generating a vector including one or more values indicative of:
one or more densities for one or more stem cell aggregates in the first spatial arrangement,
one or more density gradients for one or more stem cell aggregates in the first spatial arrangement,
one or more higher-order derivative of densities for one or more stem cell aggregates in the first spatial arrangement,
an image of the first spatial arrangement,
one or more Fourier transform components of the image of the first spatial arrangement,
when the first spatial arrangement includes at least one bead,
position of one or more beads within the first spatial arrangement, and/or
type of the one or more beads within the first spatial arrangement, and/or
one or more shapes of one or more stem cell aggregates in the first spatial arrangement.

4. The method of claim 2, wherein generating the first feature vector comprises generating a vector including:
one or more density values computed, at one or more respective length scales and using information specifying the first spatial arrangement of stem cell aggregates, for one or more stem cell aggregates in the first spatial arrangement of stem cell aggregates; and/or
one or more density gradient values computed, at one or more respective length scales and using information specifying the first spatial arrangement of stem cell aggregates, for one or more stem cell aggregates in the first spatial arrangement of stem cell aggregates.

5. The method of claim 4, wherein generating the first feature vector comprises generating a vector having a density gradient value computed at a length scale between 50 and 300 microns, optionally 200 microns, and/or a density value computed at a length scale between 500 and 1000 microns, optionally 700 microns.

6. The method of claim 2, wherein generating the first patterning metric using the trained ML model comprises generating one or more values indicative of a predicted expression for each of one or more biomarkers associated with patterned tissue to be generated using stem cell aggregates having the first spatial arrangement.

7. The method of claim 2, wherein generating the first patterning metric comprises generating one or more values indicative of:
a polarization of predicted expression of a biomarker,
a dipole moment of the predicted expression of the biomarker,
a multipole moment of the predicted expression of the biomarker,
a spatiotemporal pattern of the expression, polarization, dipole moment, and/or multipole moment of the predicted expression of the biomarker,
predicted expression and/or predicted pattern of expression of cell signals (e.g., secreted biomarker(s)), and/or
morphology of the first stem cell aggregate and/or tissue to be generated using stem cell aggregates having the first spatial arrangement.

8. The method of any one of claims 1-7, wherein the trained ML model is trained to predict, using the feature vector generated for a particular stem cell aggregate in the input spatial arrangement of stem cell aggregates, a respective patterning metric for the particular stem cell aggregate.

9. The method of any one of claims 1-7, wherein the trained ML model is trained to predict, using the feature vector generated for the input spatial arrangement of stem cell aggregates, a respective patterning metric for each of multiple stem cell aggregates in the input spatial arrangement of stem cell aggregates.

10. The method of any one of claims 1-9, wherein the trained ML model comprises a regression model.

11. The method of claim 10, wherein the regression model comprises a linear regression model.

12. The method of claim 10, wherein the regression model comprises a non-linear regression model.

13. The method of claim 12, wherein the non-linear regression model comprises a kernel regression model, optionally wherein the kernel regression model uses a linear or radial basis function (RBF) kernel.

14. The method of claim 10, wherein the regression model comprises a support vector machine (SVM) regression model, optionally wherein the SVM regression model uses a linear or RBF kernel.

15. The method of claim 10, wherein the regression model comprises a neural network model.

16. The method of claim 15, wherein the neural network model has a single layer or multilayer architecture, optionally a convolutional neural network architecture.

17. The method of any one of claims 1-16, further comprising:
obtaining training data comprising, for each particular spatial arrangement of multiple spatial arrangements,
(i) at least one measured value indicative of a patterning metric for the particular spatial arrangement,
(ii) a feature vector generated from information specifying the particular spatial arrangement; and
estimating values of parameters of an ML model using the obtained training data to obtain the trained ML model.

18. The method of claim 17, further comprising:
generating the training data at least in part by:
generating multiple random patterns;
culturing stem cell aggregates on one or multiple two-dimensional substrates and/or or in a three-dimensional matrix in accordance with the generated respective multiple random patterns;
obtaining images of the cultured stem cell aggregates; and
generating the training data from the obtained images by:
generating, from one or more of the obtained images, patterning metric values for spatial arrangements in the one or more of the obtained images; and
generating, from the one or more of the obtained images, feature vectors for spatial arrangements in the one or more of the obtained images.

19. The method of claim 18, wherein obtaining the images of the cultured stem cell aggregates comprises obtaining one or more antibody-stained, in situ probe-hybridized RNA, and/or live fluorescent reporter images of the cultured stem cell aggregates corresponding to each of one or more biomarkers.

20. The method of any one of claims 1-19, wherein using the stochastic optimization technique comprises:
obtaining a first spatial arrangement of stem cell aggregates and determining a first patterning metric for the first spatial arrangement using the trained ML model;
modifying the first spatial arrangement to generate a second spatial arrangement of stem cell aggregates different from the first spatial arrangement;
determining a second patterning metric for the second spatial arrangement using the trained ML model; and
evaluating the second patterning metric relative to the first patterning metric.

21. The method of claim 20, wherein using the stochastic optimization technique comprises,
obtaining a plurality of patterning metrics by iteratively:
generating a spatial arrangement from a previously generated spatial arrangement, and
determining a patterning metric for the spatial arrangement; and
identifying, from among the plurality of patterning metrics, one or more optimized patterning metrics.

22. The method of claim 20 or 21, wherein using the stochastic optimization technique comprises using simulated annealing with acceptance defined as a function of output of the trained ML model.

23. The method of claim 19 or 20, wherein using the stochastic optimization technique comprises using an iterative descent algorithm, optionally a gradient descent algorithm, with a step direction being a function of output of the trained ML model.

24. A system, comprising:
at least one computer hardware processor; and
at least one non-transitory computer readable storage medium storing processor executable instructions that, when executed by the at least one computer hardware processor, cause the at least one computer hardware processor to perform the method of any one of claims 1-17 and 19-23.

25. At least one non-transitory computer readable storage medium storing processor executable instructions that, when executed by at least one computer hardware processor, cause the at least one computer hardware processor to perform the method of any one of claims 1-17 and 19-23.

26. A bioengineering method for producing a target patterned tissue (e.g., human tissue), the method comprising:
coupling organoids to each other based on their relative spatial arrangement, optionally in one, two, or three dimensions; and
culturing the organoids under conditions that promote development of the target patterned tissue.

27. The bioengineering method of claim 26, wherein the spatial arrangement is determined using the method of any one of claims 1-25.

28. The bioengineering method of claim 26 or 27, wherein the organoids are derived from stem cells micropatterned on a biocompatible substrate in a predetermined spatial arrangement, and wherein the predetermined spatial arrangement promotes development of organoids into the target patterned tissue.

29. The bioengineering method of claim 28, wherein the stem cells are animal stem cells.

30. The bioengineering method of claim 29, wherein the animal stem cells comprise human stem cells or chimeric human/non-human cells.

31. The bioengineering method of claim 28 or 30, wherein the stem cells are selected from pluripotent stem cells, multipotent stem cells, oligopotent stem cells, and unipotent stem cells.

32. The bioengineering method of any one of claims 28-31, wherein the stem cells are induced pluripotent stem cells (iPSCs).

33. The bioengineering method of any one of claims 28-31, wherein the stem cells are embryonic stem cells (ESCs).

34. The bioengineering method of any one of claims 28-31, wherein the stem cells are selected from hematopoietic stem cells (HSCs), mesenchymal stem cells (MSCs), intestinal stem cells (ISCs), neuronal stem cells (NSCs), ectodermal stem cells, mesodermal stem cells, endodermal stem cells, or extraembryonic stem cells.

35. The bioengineering method of any one of claims 28-34, wherein the biocompatible substrate comprises polystyrene, borosilicate glass, poly-1-lysine, fibronectin, collagen, laminin, gelatin, Matrigel, agarose, alginate, polyethylene glycol, silicone and/or polydimethylsiloxane (PDMS).

36. The bioengineering method of any one of claims 28-33, wherein the predetermined spatial arrangement is a geometric pattern.

37. The bioengineering method of claim 36, wherein the geometric pattern is a triangular, quadrilateral, pentagonal, hexagonal, heptagonal, octagonal, nonagonal, or decagonal.

38. The bioengineering method of claim 36 or 37, wherein the geometric pattern includes a region for a central organoid.

39. The bioengineering method of any one of the claims 28-38, wherein the stem cells from which each organoid is derived are deposited on the substrate in a polygonal shape, optionally a mathematically parametrized polygonal shape, such as a trapezoid parametrized by the lengths of the parallel edges and spacing between these edges.

40. The bioengineering method of any one of the claims 26-39, wherein the spatial arrangement of organoids includes an arrangement of organoids having different shapes (e.g., a mixture of different shapes of organoids).

41. The bioengineering method of any one of claims 26-40, wherein the distance between the center of any two or more of the organoids on a biocompatible substrate is about 100 microns to about 1000 microns.

42. The bioengineering method of any one of claims 26-41, wherein the diameter, minimum length, or minimum width of each of the organoids is about 100 microns to about 1000 microns, or wherein the area occupied by each of the organoids is about 5,000 μm² to about 500,000 μm².

43. The bioengineering method of any one of claims 26-42, wherein the organoid is a brain organoid, an intestinal organoid, a liver organoid, a pancreatic organoid, a kidney organoid, a breast organoid, a lung organoid, or a heart organoid, or a gastruloid with multiple germ layer tissues that are axially and/or mediolaterally and/or dorsoventrally patterned, or an organoid derived from ectodermal, mesodermal, endodermal, and/or extraembryonic progenitors.

44. The bioengineering method of any one of claims 16-27, wherein the organoid is derived from ectodermal, mesodermal or endodermal progenitors.

45. The bioengineering method of any one of claims 26-44, wherein the conditions that promote development of the target patterned tissue include culture and/or differentiation conditions, wherein the differentiation conditions include one or more of basal medium, serum, growth factors, cytokines, supplements, optionally selected from retinoic acid (RA), morphogens including bone morphogenetic protein (BMP), fibroblast growth factor (FGF), transforming growth factor beta (TGFβ), NODAL, WNT, sonic hedgehog (SHH), or equivalent small molecule agonists and/or antagonists of these pathways, and/or vitamins, hormones, antibiotics, antimycotics, buffers, pH adjusters, pH indicators, and attachment factors, optionally selected from collagen, fibronectin, laminin, and Matrigel™.

46. The bioengineering method of any one of claims 28-45, further comprising:
culturing the micropatterned stem cells on the biocompatible substrate to form organoids; and
exposing the organoids to extracellular matrix protein and differentiation conditions.

47. The bioengineering method of claim 30 further comprising differentiating the organoids into the target patterned tissue.

48. The bioengineering method of claim 47, further comprising removing the organoids from the patterned substrate and culturing them under low-adherent conditions (e.g., on a low-adherent single-well or multi-well plate), optionally for multiple days, weeks, or months.

49. The bioengineering method of any one of claims 26-48, further comprising performing single-cell RNA sequencing, spatial transcriptomic profiling, in situ hybridization or immunostaining on cells from the organoids.

50. The bioengineering method of any one of claims 26-49, further comprising perturbing gene expression and/or function in cells of the organoids, optionally prior to development of the target patterned tissue, and assessing changes in development of the organoids into the target patterned tissue, relative to control (e.g., unperturbed) organoids.

51. The bioengineering method of claim 50, wherein the perturbing gene expression and/or function comprises delivering gene editing molecules to cells of the organoids.

52. The bioengineering method of claim 50 or 51, comprises a transfer of the cells of the organoids (optionally wherein the cells are in suspension) to wells of a multiwell (e.g., 96 well) plate and perturbing gene expression and/or function in the cells.

53. The bioengineering method of claim 51 or 52, wherein the gene editing molecules are selected from (i) programmable nucleases, optionally, a CRISPR based enzyme (e.g., CRISPR, CRISPRi, CRISPRa), a zinc finger nuclease (ZFN), a transcription activator-like effector nuclease (TALEN), or a meganuclease, and (ii) RNA interference molecules.

54. The bioengineering method of claim 53, wherein the gene editing molecules are selected from viral vectors, optionally adenovirus, adeno-associated virus (AAV) vectors, lentivirus vectors, retrovirus vectors, and herpes simplex virus (HSV) vectors, wherein the viral vectors comprise and/or express a molecule that perturbs genetic expression (e.g., gRNA or RNAi).

55. The bioengineering method of any one of claims 50-54, wherein the perturbing gene expression and/or function comprises delivering a protein agonist or antagonist to cells of the organoids.

56. The bioengineering method of any one of claims 26-55, further comprising exposing cells of the organoids to a drug or other chemical agent, optionally gradients of the drug or other chemical agent.

57. The bioengineering method of any one of claims 26-55, further comprising exposing cells of the organoids (optionally wherein the cells are in suspension) to a drug or other chemical agent, optionally gradients of the drug or other chemical agent, prior to development of the target patterned tissue, and assessing changes in development of the organoids into the target patterned tissue, relative to control organoids.

58. The bioengineering method of claim 57, further comprises transfer of the organoids to a multiwell (e.g., 96 well) plate and exposing cells of the organoids to a drug or other chemical agent.

59. The bioengineering method of any one of claims 26-58, further comprising exposing cells of the organoids to a drug or other chemical agent, optionally gradients of the drug or other chemical agent, after development of the target patterned tissue, and assessing changes in one or more characteristic of the organoids of the target patterned tissue, relative to control organoids.

60. The bioengineering method of any one of claims 56-59, wherein the drug or other chemical agent is selected from small molecule drugs (e.g., <900 daltons) and antibodies (including antibody fragments).

61. A cell micropatterning method, comprising:
micropatterning stem cells on a biocompatible substrate, in a predetermined spatial arrangement, wherein the predetermined spatial arrangement promotes development of organoids into a target patterned tissue, wherein the spatial arrangement is determined using the method of any one of claims 1-25.

62. The method of claim 61, wherein the substrate comprises polystyrene, poly-1-lysine, fibronectin, collagen, gelatin, Matrigel, polyethylene glycol, silicone and/or polydimethylsiloxane (PDMS).

63. The method of claim 62, wherein the substrate is patterned with capture molecules (e.g., DNA, RNA or protein) capable of binding to complementary or cognate target molecules in the stem cells.

64. The method of claim 61 or 62, comprising:
using photolithography to fabricate the substrate with microwells to hold signal-secreting beads at desired locations;
using a stamp (optionally a three-layer stamp) comprising water channels and a repeating micropattern to microprint shapes (e.g., circles or polygonal shapes, such as trapezoids; e.g., about 50 to 150 shapes) of extracellular matrix protein on the substrate, wherein the repeating micropattern on the stamp is aligned between the microwells;
depositing stem cells onto the substrate; and
exposing the stem cells to media containing extracellular matrix protein to produce cysts, each enclosing a respective lumen.

65. The method of claim 64 further comprising selectively depositing one or more signal-secreting or inhibitor-secreting beads into the microwells, thereby exposing the cysts to signaling gradients on the substrate, and optionally removing and/or redepositing the beads at different times.

66. The method of claim 65, wherein the beads are selectively injected into the microwells using a manual injector featuring a glass capillary controlled by pneumatic pressure.

67. The method of claim 64 or 65, wherein placement of the one or more beads in the microwells relative to the cysts is determined using the method of any one of claims 1-25.

68. A method of producing one or more organoids comprising:
seeding a population of pluripotent stem cells on a surface in a repeating pattern; and
differentiating the deposited pluripotent stem cells to form one or more organoids.

69. A method of producing one or more organoids comprising:
seeding a population of pluripotent stem cells on a micropatterned substrate in a repeating pattern; and
differentiating the deposited pluripotent stem cells to form one or more organoids.

70. A high throughput method of perturbing organoids comprising:
preparing lentivirus in a multiwell (optionally a 6, 12, 24, 96, or 384 well) plate, wherein each well of the multiwell plate comprises an agent for genetic perturbation (e.g., knock down, knockout, or overexpression);
printing one or more patterns on a surface;
placing a removable well system on the surface, thereby separating the one or more patterns from one another;
combining the lentivirus with stem cells to form a mixed cell suspension;
depositing the mixed cell suspension on the one or more patterns;
differentiating deposited cells of the suspension to form one or more organoids; and
assessing changes in development of, or in one or more characteristic of, the organoids.

71. A rapid, high throughput screening method, comprising:
on a biocompatible multiwell substrate or on multiple biocompatible multiwell substrates, coupling organoids to each other based on their relative spatial arrangement, optionally wherein the substrate is fabricated using the method of any one of claims 61-67;
culturing the organoids under conditions that promote development of a target patterned tissue;
exposing the organoids to cell signals, or to genetic or chemical perturbation; and
assessing changes in development of, or in one or more characteristic of, the organoids.

72. The screening method of claim 71, wherein each of the biocompatible multiwell substrates comprises a repeating micropattern of extracellular matrix protein and organoids.

73. The method of claim 71 or 72, wherein exposing the organoids to cell signals or chemical perturbation comprises depositing signal-secreting beads or chemical-coated beads, respectively, into the microwells.

74. The method of claim 71 or 72, wherein exposing the organoids to genetic perturbation comprises delivering gene editing agents to the organoids.
